SEQUENCE LISTING (196 sequences)

SEQ ID NO: 1 - protein, 10 aa, synthetic construct
  REGION 1..10: note = Peptide
ASEPRPFWGY

SEQ ID NO: 2 - protein, 10 aa, synthetic construct
  REGION 1..10: note = Peptide
GSDRGLYLEY

SEQ ID NO: 3 - protein, 9 aa, synthetic construct
  REGION 1..9: note = Peptide
ALIKHVANA

SEQ ID NO: 4 - protein, 9 aa, synthetic construct
  REGION 1..9: note = Peptide
IMGEFRTEV

SEQ ID NO: 5 - protein, 9 aa, synthetic construct
  REGION 1..9: note = Peptide
MVAESPPRV

SEQ ID NO: 6 - protein, 9 aa, synthetic construct
  REGION 1..9: note = Peptide
RLATHIDGA

SEQ ID NO: 7 - protein, 9 aa, synthetic construct
  REGION 1..9: note = Peptide
RLLQETHQA

SEQ ID NO: 8 - protein, 10 aa, synthetic construct
  REGION 1..10: note = Peptide
AMVAESPPRV

SEQ ID NO: 9 - protein, 9 aa, synthetic construct
  REGION 1..9: note = Peptide
LTEKELLNY

SEQ ID NO: 10 - protein, 9 aa, synthetic construct
  REGION 1..9: note = Peptide
MESHSVAQA

SEQ ID NO: 11 - protein, 11 aa, synthetic construct
  REGION 1..11: note = Peptide
SEVDSVRDRLP

SEQ ID NO: 12 - protein, 8 aa, synthetic construct
  REGION 1..8: note = Peptide
TEIEAIHF

SEQ ID NO: 13 - protein, 9 aa, synthetic construct
  REGION 1..9: note = Peptide
AEEERRPPA

SEQ ID NO: 14 - protein, 9 aa, synthetic construct
  REGION 1..9: note = Peptide
AEKPPGSVA

SEQ ID NO: 15 - protein, 9 aa, synthetic construct
  REGION 1..9: note = Peptide
AESAIPVRT

SEQ ID NO: 16 - protein, 9 aa, synthetic construct
  REGION 1..9: note = Peptide
MEATHSTLP

SEQ ID NO: 17 - protein, 9 aa, synthetic construct
  REGION 1..9: note = Peptide
MESHFVAQA

SEQ ID NO: 18 - protein, 9 aa, synthetic construct
  REGION 1..9: note = Peptide
HRARSIFIF

SEQ ID NO: 19 - protein, 9 aa, synthetic construct
  REGION 1..9: note = Peptide
THAKSFLVF

SEQ ID NO: 20 - protein, 8 aa, synthetic construct
  REGION 1..8: note = Peptide
EETYFHLF

SEQ ID NO: 21 - protein, 9 aa, synthetic construct
  REGION 1..9: note = Peptide
MEERVVRIA

SEQ ID NO: 22 - protein, 11 aa, synthetic construct
  REGION 1..11: note = Peptide
AEAGELLEPGR

SEQ ID NO: 23 - protein, 11 aa, synthetic construct
  REGION 1..11: note = Peptide
GMQGPPPGQVP

SEQ ID NO: 24 - protein, 12 aa, synthetic construct
  REGION 1..12: note = Peptide
EAGAGAVEGRVA

SEQ ID NO: 25 - protein, 8 aa, synthetic construct
  REGION 1..8: note = Peptide
LCDSLPRY

SEQ ID NO: 26 - protein, 9 aa, synthetic construct
  REGION 1..9: note = Peptide
VSDHPALLY

SEQ ID NO: 27 - protein, 9 aa, synthetic construct
  REGION 1..9: note = Peptide
LTEKELLNY

SEQ ID NO: 28 - protein, 10 aa, synthetic construct
  REGION 1..10: note = Peptide
GSDRGLYLEY

SEQ ID NO: 29 - protein, 10 aa, synthetic construct
  REGION 1..10: note = Peptide
ASEPRPFWGY

SEQ ID NO: 30 - protein, 10 aa, synthetic construct
  REGION 1..10: note = Peptide
SISSPTLLLY

SEQ ID NO: 31 - protein, 11 aa, synthetic construct
  REGION 1..11: note = Peptide
MSSGMTKDSLY

SEQ ID NO: 32 - protein, 11 aa, synthetic construct
  REGION 1..11: note = Peptide
VMGLLDLSERY

SEQ ID NO: 33 - protein, 11 aa, synthetic construct
  REGION 1..11: note = Peptide
ASDYGSEDSRY

SEQ ID NO: 34 - protein, 12 aa, synthetic construct
  REGION 1..12: note = Peptide
ATEHDKNSTNIY

SEQ ID NO: 35 - protein, 12 aa, synthetic construct
  REGION 1..12: note = Peptide
YSETSLSNGGKY

SEQ ID NO: 36 - protein, 9 aa, synthetic construct
  REGION 1..9: note = Peptide
RLATHIDGA

SEQ ID NO: 37 - protein, 9 aa, synthetic construct
  REGION 1..9: note = Peptide
HLLDVPVVL

SEQ ID NO: 38 - protein, 9 aa, synthetic construct
  REGION 1..9: note = Peptide
RLLQETHQA

SEQ ID NO: 39 - protein, 9 aa, synthetic construct
  REGION 1..9: note = Peptide
HLIQEEIIL

SEQ ID NO: 40 - protein, 9 aa, synthetic construct
  REGION 1..9: note = Peptide
ALAEVFHQL

SEQ ID NO: 41 - protein, 9 aa, synthetic construct
  REGION 1..9: note = Peptide
IMGEFRTEV

SEQ ID NO: 42 - protein, 9 aa, synthetic construct
  REGION 1..9: note = Peptide
GVVSLFVGL

SEQ ID NO: 43 - protein, 9 aa, synthetic construct
  REGION 1..9: note = Peptide
HLLQKELIL

SEQ ID NO: 44 - protein, 10 aa, synthetic construct
  REGION 1..10: note = Peptide
VMLESPPKGV

SEQ ID NO: 45 - protein, 10 aa, synthetic construct
  REGION 1..10: note = Peptide
KILDLVSGAL

SEQ ID NO: 46 - protein, 8 aa, synthetic construct
  REGION 1..8: note = Peptide
EETYFHLF

SEQ ID NO: 47 - protein, 8 aa, synthetic construct
  REGION 1..8: note = Peptide
EHIILNTL

SEQ ID NO: 48 - protein, 8 aa, synthetic construct
  REGION 1..8: note = Peptide
MESPSVRP

SEQ ID NO: 49 - protein, 9 aa, synthetic construct
  REGION 1..9: note = Peptide
AEKPPGSVA

SEQ ID NO: 50 - protein, 9 aa, synthetic construct
  REGION 1..9: note = Peptide
AEAGRSLEA

SEQ ID NO: 51 - protein, 9 aa, synthetic construct
  REGION 1..9: note = Peptide
RESKHIHAP

SEQ ID NO: 52 - protein, 9 aa, synthetic construct
  REGION 1..9: note = Peptide
EEHQKMNKP

SEQ ID NO: 53 - protein, 9 aa, synthetic construct
  REGION 1..9: note = Peptide
AEARGSPEV

SEQ ID NO: 54 - protein, 9 aa, synthetic construct
  REGION 1..9: note = Peptide
MESHSVAQA

SEQ ID NO: 55 - protein, 9 aa, synthetic construct
  REGION 1..9: note = Peptide
SEEPLRLAA

SEQ ID NO: 56 - protein, 9 aa, synthetic construct
  REGION 1..9: note = Peptide
AEVGRSPEV

SEQ ID NO: 57 - protein, 9 aa, synthetic construct
  REGION 1..9: note = Peptide
AERFSTLTA

SEQ ID NO: 58 - protein, 9 aa, synthetic construct
  REGION 1..9: note = Peptide
MEERVVRIA

SEQ ID NO: 59 - protein, 9 aa, synthetic construct
  REGION 1..9: note = Peptide
RESRVTPPP

SEQ ID NO: 60 - protein, 9 aa, synthetic construct
  REGION 1..9: note = Peptide
AETGSHYVA

SEQ ID NO: 61 - protein, 9 aa, synthetic construct
  REGION 1..9: note = Peptide
TEMWDLRAP

SEQ ID NO: 62 - protein, 9 aa, synthetic construct
  REGION 1..9: note = Peptide
MEFHSVAQA

SEQ ID NO: 63 - protein, 11 aa, synthetic construct
  REGION 1..11: note = Peptide
QEDPSSHLIIA

SEQ ID NO: 64 - protein, 9 aa, synthetic construct
  REGION 1..9: note = Peptide
KIDSESIIL

SEQ ID NO: 65 - protein, 9 aa, synthetic construct
  REGION 1..9: note = Peptide
FQPNNFQKY

SEQ ID NO: 66 - protein, 9 aa, synthetic construct
  REGION 1..9: note = Peptide
ISHDSLVLL

SEQ ID NO: 67 - protein, 9 aa, synthetic construct
  REGION 1..9: note = Peptide
SFKSNGLSM

SEQ ID NO: 68 - protein, 8 aa, synthetic construct
  REGION 1..8: note = Peptide
KTQQAHTA

SEQ ID NO: 69 - protein, 8 aa, synthetic construct
  REGION 1..8: note = Peptide
THYDSIYK

SEQ ID NO: 70 - protein, 8 aa, synthetic construct
  REGION 1..8: note = Peptide
ISDFFCCC

SEQ ID NO: 71 - protein, 8 aa, synthetic construct
  REGION 1..8: note = Peptide
ATRDLHTA

SEQ ID NO: 72 - protein, 8 aa, synthetic construct
  REGION 1..8: note = Peptide
KNRHLLEL

SEQ ID NO: 73 - protein, 8 aa, synthetic construct
  REGION 1..8: note = Peptide
WLTRPVGV

SEQ ID NO: 74 - protein, 9 aa, synthetic construct
  REGION 1..9: note = Peptide
HHHHHPSSS

SEQ ID NO: 75 - protein, 9 aa, synthetic construct
  REGION 1..9: note = Peptide
PCVETPSQR

SEQ ID NO: 76 - protein, 10 aa, synthetic construct
  REGION 1..10: note = Peptide
HHHHHHPSSS

SEQ ID NO: 77 - protein, 10 aa, synthetic construct
  REGION 1..10: note = Peptide
LHLLLFLVIV

SEQ ID NO: 78 - protein, 11 aa, synthetic construct
  REGION 1..11: note = Peptide
LTKSTNILYII

SEQ ID NO: 79 - protein, 11 aa, synthetic construct
  REGION 1..11: note = Peptide
QASHVPGAAAG

SEQ ID NO: 80 - protein, 10 aa, synthetic construct
  REGION 1..10: note = Peptide
SLDVGGPLRY

SEQ ID NO: 81 - protein, 10 aa, synthetic construct
  REGION 1..10: note = Peptide
WILMESCINF

SEQ ID NO: 82 - protein, 9 aa, synthetic construct
  REGION 1..9: note = Peptide
LTEKELLNY

SEQ ID NO: 83 - protein, 8 aa, synthetic construct
  REGION 1..8: note = Peptide
TEIEAIHF

SEQ ID NO: 84 - protein, 10 aa, synthetic construct
  REGION 1..10: note = Peptide
ESDVSSALSY

SEQ ID NO: 85 - protein, 9 aa, synthetic construct
  REGION 1..9: note = Peptide
CWIKWENAY

SEQ ID NO: 86 - protein, 10 aa, synthetic construct
  REGION 1..10: note = Peptide
ALYEKDNTYL

SEQ ID NO: 87 - protein, 10 aa, synthetic construct
  REGION 1..10: note = Peptide
TFSPCHFFLT

SEQ ID NO: 88 - protein, 8 aa, synthetic construct
  REGION 1..8: note = Peptide
ATRDLHTA

SEQ ID NO: 89 - protein, 10 aa, synthetic construct
  REGION 1..10: note = Peptide
LSSYHLAHVI

SEQ ID NO: 90 - protein, 9 aa, synthetic construct
  REGION 1..9: note = Peptide
TFFFNLEEF

SEQ ID NO: 91 - protein, 9 aa, synthetic construct
  REGION 1..9: note = Peptide
QNTREIFTY

SEQ ID NO: 92 - protein, 8 aa, synthetic construct
  REGION 1..8: note = Peptide
EETYFHLF

SEQ ID NO: 93 - protein, 8 aa, synthetic construct
  REGION 1..8: note = Peptide
LMEHIHKL

SEQ ID NO: 94 - protein, 9 aa, synthetic construct
  REGION 1..9: note = Peptide
RESRVTPPP

SEQ ID NO: 95 - protein, 11 aa, synthetic construct
  REGION 1..11: note = Peptide
QEDPSSHLIIA

SEQ ID NO: 96 - protein, 9 aa, synthetic construct
  REGION 1..9: note = Peptide
EETGTAISP

SEQ ID NO: 97 - protein, 9 aa, synthetic construct
  REGION 1..9: note = Peptide
AEERWEVVL

SEQ ID NO: 98 - protein, 9 aa, synthetic construct
  REGION 1..9: note = Peptide
AEAEGSLGP

SEQ ID NO: 99 - protein, 9 aa, synthetic construct
  REGION 1..9: note = Peptide
AEAGRSPEV

SEQ ID NO: 100 - protein, 10 aa, synthetic construct
  REGION 1..10: note = Peptide
ASEPRPFWGY

SEQ ID NO: 101 - protein, 9 aa, synthetic construct
  REGION 1..9: note = Peptide
ILMEHIHKL

SEQ ID NO: 102 - protein, 8 aa, synthetic construct
  REGION 1..8: note = Peptide
DTIEVAEV

SEQ ID NO: 103 - protein, 9 aa, synthetic construct
  REGION 1..9: note = Peptide
ITNKYQLVF

SEQ ID NO: 104 - protein, 9 aa, synthetic construct
  REGION 1..9: note = Peptide
IRRENIRFL

SEQ ID NO: 105 - protein, 9 aa, synthetic construct
  REGION 1..9: note = Peptide
HLIQEEIIL

SEQ ID NO: 106 - protein, 9 aa, synthetic construct
  REGION 1..9: note = Peptide
AEKPPGSVA

SEQ ID NO: 107 - protein, 11 aa, synthetic construct
  REGION 1..11: note = Peptide
ILMEHIHKLKA

SEQ ID NO: 108 - protein, 9 aa, synthetic construct
  REGION 1..9: note = Peptide
STDGIGKSY

SEQ ID NO: 109 - protein, 9 aa, synthetic construct
  REGION 1..9: note = Peptide
TLCDLYETL

SEQ ID NO: 110 - protein, 9 aa, synthetic construct
  REGION 1..9: note = Peptide
MEERVVRIA

SEQ ID NO: 111 - protein, 8 aa, synthetic construct
  REGION 1..8: note = Peptide
ASGPPVTL

SEQ ID NO: 112 - protein, 9 aa, synthetic construct
  REGION 1..9: note = Peptide
AEVGRSLEA

SEQ ID NO: 113 - protein, 10 aa, synthetic construct
  REGION 1..10: note = Peptide
KLLDPRIYYI

SEQ ID NO: 114 - protein, 8 aa, synthetic construct
  REGION 1..8: note = Peptide
KTQQAHTA

SEQ ID NO: 115 - protein, 9 aa, synthetic construct
  REGION 1..9: note = Peptide
MEFHSVAQA

SEQ ID NO: 116 - protein, 9 aa, synthetic construct
  REGION 1..9: note = Peptide
ISHDSLVLL

SEQ ID NO: 117 - protein, 9 aa, synthetic construct
  REGION 1..9: note = Peptide
MILLKLTSV

SEQ ID NO: 118 - protein, 9 aa, synthetic construct
  REGION 1..9: note = Peptide
ELDLKATQA

SEQ ID NO: 119 - protein, 9 aa, synthetic construct
  REGION 1..9: note = Peptide
KRFESWRVL

SEQ ID NO: 120 - protein, 8 aa, synthetic construct
  REGION 1..8: note = Peptide
DTSWPLSL

SEQ ID NO: 121 - protein, 9 aa, synthetic construct
  REGION 1..9: note = Peptide
MERRDIFTV

SEQ ID NO: 122 - protein, 9 aa, synthetic construct
  REGION 1..9: note = Peptide
SEEPLRLAA

SEQ ID NO: 123 - protein, 9 aa, synthetic construct
  REGION 1..9: note = Peptide
EEHQKMNKP

SEQ ID NO: 124 - protein, 9 aa, synthetic construct
  REGION 1..9: note = Peptide
RESKHIHAP

SEQ ID NO: 125 - protein, 8 aa, synthetic construct
  REGION 1..8: note = Peptide
LSRTRKGP

SEQ ID NO: 126 - protein, 9 aa, synthetic construct
  REGION 1..9: note = Peptide
AEVGRSPEV

SEQ ID NO: 127 - protein, 10 aa, synthetic construct
  REGION 1..10: note = Peptide
LSLSLSLQFS

SEQ ID NO: 128 - protein, 9 aa, synthetic construct
  REGION 1..9: note = Peptide
LLDPRIYYI

SEQ ID NO: 129 - protein, 8 aa, synthetic construct
  REGION 1..8: note = Peptide
AEERNLYV

SEQ ID NO: 130 - protein, 9 aa, synthetic construct
  REGION 1..9: note = Peptide
MESHSVAQA

SEQ ID NO: 131 - protein, 9 aa, synthetic construct
  REGION 1..9: note = Peptide
DLLLKETQL

SEQ ID NO: 132 - protein, 8 aa, synthetic construct
  REGION 1..8: note = Peptide
YEINAHKY

SEQ ID NO: 133 - protein, 10 aa, synthetic construct
  REGION 1..10: note = Peptide
EAERGESLDS

SEQ ID NO: 134 - protein, 9 aa, synthetic construct
  REGION 1..9: note = Peptide
TEAEAHLQA

SEQ ID NO: 135 - protein, 9 aa, synthetic construct
  REGION 1..9: note = Peptide
MQIFVKTLT

SEQ ID NO: 136 - protein, 9 aa, synthetic construct
  REGION 1..9: note = Peptide
AETGSHYVA

SEQ ID NO: 137 - protein, 12 aa, synthetic construct
  REGION 1..12: note = Peptide
PIVNTFNVFCNS

SEQ ID NO: 138 - protein, 8 aa, synthetic construct
  REGION 1..8: note = Peptide
PEIKLHTY

SEQ ID NO: 139 - protein, 8 aa, synthetic construct
  REGION 1..8: note = Peptide
EEMPLNVA

SEQ ID NO: 140 - protein, 9 aa, synthetic construct
  REGION 1..9: note = Peptide
NLDPAVHEV

SEQ ID NO: 141 - protein, 9 aa, synthetic construct
  REGION 1..9: note = Peptide
HCIQDNIYY

SEQ ID NO: 142 - protein, 10 aa, synthetic construct
  REGION 1..10: note = Peptide
QSLSARGSAP

SEQ ID NO: 143 - protein, 9 aa, synthetic construct
  REGION 1..9: note = Peptide
ETFRSCSFV

SEQ ID NO: 144 - protein, 8 aa, synthetic construct
  REGION 1..8: note = Peptide
KNVFSQEL

SEQ ID NO: 145 - protein, 9 aa, synthetic construct
  REGION 1..9: note = Peptide
IMGEFRTEV

SEQ ID NO: 146 - protein, 9 aa, synthetic construct
  REGION 1..9: note = Peptide
IICMSFVNF

SEQ ID NO: 147 - protein, 9 aa, synthetic construct
  REGION 1..9: note = Peptide
NSISKKKKN

SEQ ID NO: 148 - protein, 12 aa, synthetic construct
  REGION 1..12: note = Peptide
ATEHDKNSTNIY

SEQ ID NO: 149 - protein, 8 aa, synthetic construct
  REGION 1..8: note = Peptide
KNRHLLEL

SEQ ID NO: 150 - protein, 9 aa, synthetic construct
  REGION 1..9: note = Peptide
HLLDVPVVL

SEQ ID NO: 151 - protein, 10 aa, synthetic construct
  REGION 1..10: note = Peptide
HHHHHHPSSS

SEQ ID NO: 152 - protein, 9 aa, synthetic construct
  REGION 1..9: note = Peptide
HHHHHPSSS

SEQ ID NO: 153 - protein, 9 aa, synthetic construct
  REGION 1..9: note = Peptide
HLLQKELIL

SEQ ID NO: 154 - protein, 9 aa, synthetic construct
  REGION 1..9: note = Peptide
AEAGGLLEP

SEQ ID NO: 155 - protein, 9 aa, synthetic construct
  REGION 1..9: note = Peptide
HLLDVPVVL

SEQ ID NO: 156 - protein, 9 aa, synthetic construct
  REGION 1..9: note = AH1 peptide
SPSYVYHQF

SEQ ID NO: 157 - protein, 9 aa, synthetic construct
  REGION 1..9: note = Peptide
RGPSHFSRL

SEQ ID NO: 158 - protein, 9 aa, synthetic construct
  REGION 1..9: note = Peptide
KYLRLHERI

SEQ ID NO: 159 - protein, 9 aa, synthetic construct
  REGION 1..9: note = Peptide
SLPVRSLSL

SEQ ID NO: 160 - protein, 9 aa, synthetic construct
  REGION 1..9: note = Peptide
SVPVRVSII

SEQ ID NO: 161 - protein, 9 aa, synthetic construct
  REGION 1..9: note = Peptide
SLPVRSLSL

SEQ ID NO: 162 - protein, 9 aa, synthetic construct
  REGION 1..9: note = Peptide
KGPEHKLLL

SEQ ID NO: 163 - protein, 9 aa, synthetic construct
  REGION 1..9: note = Peptide
RGPSLSRDL

SEQ ID NO: 164 - protein, 9 aa, synthetic construct
  REGION 1..9: note = Peptide
SHPQKYERI

SEQ ID NO: 165 - protein, 8 aa, synthetic construct
  REGION 1..8: note = Peptide
TGPRRPQI

SEQ ID NO: 166 - protein, 9 aa, synthetic construct
  REGION 1..9: note = Peptide
RLAQLQTTI

SEQ ID NO: 167 - protein, 9 aa, synthetic construct
  REGION 1..9: note = Peptide
RGPLLEKLF

SEQ ID NO: 168 - protein, 9 aa, synthetic construct
  REGION 1..9: note = Peptide
GFLGSNTDI

SEQ ID NO: 169 - protein, 11 aa, synthetic construct
  REGION 1..11: note = Peptide
HPTVPNPYNLL

SEQ ID NO: 170 - protein, 9 aa, synthetic construct
  REGION 1..9: note = Peptide
GSPSLVHQV

SEQ ID NO: 171 - protein, 9 aa, synthetic construct
  REGION 1..9: note = Peptide
SNISYKNGI

SEQ ID NO: 172 - protein, 10 aa, synthetic construct
  REGION 1..10: note = Peptide
RPLISIKHGL

SEQ ID NO: 173 - protein, 9 aa, synthetic construct
  REGION 1..9: note = Peptide
LNPSALSAL

SEQ ID NO: 174 - protein, 9 aa, synthetic construct
  REGION 1..9: note = Peptide
RATPEVTLI

SEQ ID NO: 175 - protein, 8 aa, synthetic construct
  REGION 1..8: note = Peptide
YNPILSKL

SEQ ID NO: 176 - protein, 8 aa, synthetic construct
  REGION 1..8: note = Peptide
HIFSLHHF

SEQ ID NO: 177 - DNA, 300 bp, synthetic construct
  source 1..300: mol_type = other DNA
  misc_feature 1..300: note = cDNA; note = Gm37283 ENSMUST00000193111.1
ccccttatcccaacatctgttactctgatgatatccagtcttatacattcatatgacatcaaatactatgaattctcaaatcatatatttagtctgcatcactttcctgaatatataagacctggttgcttagttggcaacttgagtaggagagataaaagatgttttaatgctagaaagagagaaaaaaaaaaaaacaaaaaaaaaaaccagaggtccaatttcttcctacaccagtttcccgtttagtgaatggcattccatcctccacttaggatggtttaggtccagctcccaagg

SEQ ID NO: 178 - DNA, 240 bp, synthetic construct
  source 1..240: mol_type = other DNA
  misc_feature 1..240: note = cDNA; note = Gm17173 ENSMUST00000168000.1
gtttcctcagagctctgtgaagtgcctcaaatcatgactctccctgctgaccttcaggtcacactgctacctagattggcacaactccagaccaccatctgaggctggatgctttttcttatggcattagacttcccctgcctactagatcctaaatccacaatgaaacaaattaactctaatggaacccaaacttcagaaatccaatattagtggaatagttgtggttaagagcactga

SEQ ID NO: 179 - DNA, 180 bp, synthetic construct
  source 1..180: mol_type = other DNA
  misc_feature 1..180: note = cDNA; note = Gm37494 ENSMUST00000188038.2
catgtagggtctgtagtaaagcctttctatgtcctcctttccttcagaggcatgaaaaaactcacgctgaggaaaagccctttgaatgtaagcaatgtagtaaaatcttcagagattggagtaccttccaagtgcatgaaagatctcacactctagaaaagccctatgcatgtaagctat

SEQ ID NO: 180 - protein, 82 aa, Mus musculus
  REGION 28..35: note = Identified MHC class I bound peptide
PLIPTSVTLMISSLIHSYDIKYYEFSNHIFSLHHFPEYIRPGCLVGNLSRRDKRCFNARKREKKKNKKKNQRSNFFLHQFPV

SEQ ID NO: 181 - protein, 10 aa, Mus musculus
MAFHPPLRMV

SEQ ID NO: 182 - protein, 5 aa, Mus musculus
VQLPR

SEQ ID NO: 183 - protein, 33 aa, Mus musculus
  REGION 25..33: note = Identified MHC class I bound peptide
VSSELCEVPQIMTLPADLQVTLLPRLAQLQTTI

SEQ ID NO: 184 - protein, 36 aa, Mus musculus
GWMLFLMALDFPCLLDPKSTMKQINSNGTQTSEIQY

SEQ ID NO: 185 - protein, 5 aa, Mus musculus
WNSCG

SEQ ID NO: 186 - protein, 51 aa, Mus musculus
  REGION 26..34: note = Identified MHC class I bound peptide
HVGSVVKPFYVLLSFRGMKKLTLRKSPLNVSNVVKSSEIGVPSKCMKDLTL

SEQ ID NO: 187 - protein, 8 aa, Mus musculus
KSPMHVSY

SEQ ID NO: 188 - DNA, 240 bp, synthetic construct
  source 1..240: mol_type = other DNA
  misc_feature 1..240: note = MALAT1 ENST00000534336.1; note = cDNA
gatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggg

SEQ ID NO: 189 - DNA, 240 bp, synthetic construct
  source 1..240: mol_type = other DNA
  misc_feature 1..240: note = cDNA; note = AC079135.1 MICT00000210263.1
gacgcggagaagccgccggggagcgtggccatgagcgtagaggcgctaattaagcacgttgcgaacgcaaggagtgctcaccctggcgccgccgcccggtttccagcgcgaggactcgagggcgcgcggttcctctttgctaactgcaggatggagccgatcccctcaggatgtttcccctgttctcgacaacgctcttgtcttccctttaaaaatggaatcacttcttgacaacggcag

SEQ ID NO: 190 - protein, 24 aa, Homo sapiens
DEGSRRLGLATTRRRRAGVVRRGS

SEQ ID NO: 191 - protein, 49 aa, Homo sapiens
  REGION 41..49: note = Identified MHC class I bound peptide
TSMPVCQGHRESEWLVKIREVGNMLFFWNLLMVTFYLFSNIMGEFRTEV

SEQ ID NO: 192 - protein, 5 aa, Homo sapiens
RDLYG

SEQ ID NO: 193 - protein, 76 aa, Homo sapiens
  REGION 15..23: note = Identified MHC class I bound peptide
DAEKPPGSVAMSVEALIKHVANARSAHPGAAARFPARGLEGARFLFANCRMEPIPSGCFPCSRQRSCLPFKNGITS

SEQ ID NO: 194 - protein, 9 aa, synthetic construct
  REGION 1..9: note = Peptide
YYIPGLKGI

SEQ ID NO: 195 - RNA, 19 nt, synthetic construct
  source 1..19: mol_type = other RNA
  misc_feature 1..19: note = siRNA
agctgaccctgaagttctt

SEQ ID NO: 196 - RNA, 21 nt, synthetic construct
  source 1..21: mol_type = other RNA
  misc_feature 1..21: note = siRNA
ctcctcgcagatcgtcatctt

(19) United States
(12) Patent Application Publication
(10) Pub. No.: US 2026/0158125 A1
(43) Pub. Date: Jun. 11, 2026

(54) PEPTIDE VACCINE

(71) Applicant: ARGONAUT THERAPEUTICS LIMITED, Oxford (GB)

(72) Inventors: Nicholas LA THANGUE, Oxford (GB); Wojciech BARCZAK, Oxford (GB); Simon Mark CARR, Oxford (GB)

(21) Appl. No.: 18/676,282

(22) PCT Filed: Nov. 28, 2022
(86) PCT No.: PCT/GB2022/053012

§ 371 Date: May 28, 2024

(30) Foreign Application Priority Data
Nov. 29, 2021 (GB) 2117230.9

(51) Int. Cl. A61K 39/00; A61K 39/385; A61P 35/00; C12N 15/113; C12N 15/86; G01N 33/50

(52) U.S. Cl. CPC A61K 39/001154; A61K 39/385; A61P 35/00; C12N 15/1137; C12N 15/86; G01N 33/5023; A61K 2039/572; A61K 2039/575; A61K 2039/605; C12N 2710/10343; C12Y 201/01

(57) ABSTRACT

The present invention provides one or more immunogenic peptides derived from a PRMT5-E2F1 axis regulated long non-coding RNA gene or a derivative thereof; a pharmaceutical composition comprising one or more of said peptides; a vaccine comprising one or more of said peptides and their use in therapy, including a method for eliciting an immune response in a mammalian subject by administration of an agent capable of presenting the peptides to the host. The invention also relates to the use of a PRMT5 inhibitor for use in treating cancer by stimulating host immunity.

### FIELD OF THE INVENTION

The present invention provides one or more immunogenic peptides and method for eliciting an immune response in a mammalian subject by administration of an agent capable of presenting the peptides to the host. In particular, the invention relates to a vaccine for therapeutic use, such as in the treatment of cancer. The invention also relates to the use of a PRMT5 inhibitor for treating cancer by stimulating host immunity.

### INTRODUCTION

The retinoblastoma protein (pRb)-E2F pathway is a key point of control in the cell cycle. It is often deregulated in tumour cells, and deregulation of the pathway is widely regarded as a ‘hallmark’ of cancer. Classically, the pRb tumour suppressor protein is a negative regulator of E2F transcription factors, which act as a transcriptional hub through which pRb exerts effects on cell cycle progression. The temporal regulation of cyclin/CDK complexes and the sequential phosphorylation of pRb releases E2F at the G1/S phase transition enabling E2F to drive the transcription of genes required for cell cycle progression. Classical E2F target genes include well characterised cell cycle, DNA synthesis and apoptotic targets, and others connected with metabolic control, differentiation, senescence and autophagy (Blanchet et al., 2011; Denchi and Helin, 2005; Kent and Leone, 2019; Munro et al., 2012; Roworth et al., 2015; Wu et al., 2001; Yao et al., 2008).

It has become apparent that the pRb-E2F pathway regulates a much larger gene network than originally envisaged, reflecting an influential ‘reader-writer’ event which occurs at sites of arginine methylation on the E2F1 subunit by the protein arginine methyltransferase (PRMT) 5 (Barczak et al., 2020; Cho et al., 2012; Zheng et al., 2013). Residue-specific methylation occurs within a central arginine (R)-rich cluster which at the biological level facilitates proliferation and cell growth (Cho et al., 2012; Zheng et al., 2013). Significantly, genome-wide analysis identified a large repertoire of genes under PRMT5-E2F1 control (Roworth et al., 2019). This occurs through p100/TSN which, by virtue of its tudor domain reads the meR mark, switching E2F1 from its primary role as a transcriptional regulator to one with a wide influence on other levels of gene expression control, including alternative RNA splicing (Roworth et al., 2019). The frequent over-expression of PRMT5 in diverse human tumours and the critical role that E2F plays in the cancer cell cycle argues strongly for the importance of the PRMT5-E2F1 axis in driving malignant disease. Much of the human genome is made up of non-classical genes, composed of different structural and regulatory elements and includes genes for example encoding microRNAs and long non-coding (Inc) RNAs (Gebert and MacRae, 2019; Statello et al., 2021).

LncRNA genes are a major source of transcription in mammalian cells, typically encoding transcripts with lengths of over 200 nucleotides, but with most of them believed to exist as untranslated RNAs (Statello et al., 2021).

Here, we describe a new and unexpected role for the PRMT5-E2F1 axis in regulating the expression of long non-coding (Inc) genes within the non-coding cancer genome. Although widely believed to be an untranslated population of RNA, we have found that a group of lncRNA transcripts can be translated and further processed into peptides. Significantly, many lncRNA derived peptides contribute to the antigenic landscape of tumour cells, where they are presented by the MHC class I protein complex to the immune system. Both PRMT5 and E2F1 influence the expression of lncRNA genes and thereby control the repertoire of peptides presented by MHC class I antigens. Pharmacological control of the PRMT5-E2F1 axis alters the profile of tumour associated antigens derived from lncRNA genes thereby influencing an effective adaptive immune response.

As described herein, we have designed a stand-alone therapeutic vaccine, composed of lncRNA derived peptide antigens, and have found that this is immunogenic and prompts an effective anti-tumour immune response. These results show that PRMT5 links E2F activity to the non-coding genome and the antigenic landscape of tumour cells. Manipulating PRMT5 activity thus offers a therapeutic strategy to control the immunogenicity of tumour cells.

Cancer vaccines and long-term tumour-specific immunity may prove useful for managing human or animal treatment of various tumours, and preventing tumour recurrence. The present invention provides new tools and methods for vaccine immunotherapy.

### DESCRIPTION OF THE DRAWINGS

FIG. 1: Differential expression analysis of lncRNA transcripts present in HCT116 cells.

A) Venn diagrams demonstrating the crossover of lncRNA transcripts up- or down-regulated at the 30% level (q<0.05) in WT or E2F1 Cr cell lines treated with T1-44 for 48 h as indicated, with respect to the WT E2F1 DMSO treated cells.

B) A bar chart to represent the total number of lncRNA transcripts that are differentially up- and down-regulated at a statistically significant level (q<0.05) in each cell line and treatment, with respect to WT E2F1 DMSO treated cells. A fold change cut-off was not applied to the data.

C) (i) WT E2F1 or E2F1 Cr cell lines were treated with 1 μM T1-44 for 48 h prior to RT-qPCR analysis to determine the expression of the indicated lncRNA transcripts (labelled with their ENSEMBL transcript name). (ii) An immunoblot is also included to display input protein levels of E2F1, and SDMe was used as a marker for T1-44 activity.

D) (i) WT E2F1 or E2F1 Cr cell lines were treated with 1 μM T1-44 for 48 h prior to chromatin extraction and ChIP analysis using an E2F1 specific antibody, or an IgG control antibody. ChIP-seq data from ENCODE was used to identify potential E2F1 binding sites (marked by a smaller red box [no arrow]) around the TSS of each lncRNA gene (marked by an arrow), and primers were designed around these sites for use in qPCR. CDC6 and actin genes were included as positive and negative controls respectively. (ii) An immunoblot is also included to display input protein levels of E2F1, and SDMe was used to indicate activity of T1-44.

FIG. 2: Differential expression analysis of lncRNA transcripts present in CT26 cells grown in vitro and in situ as tumours

A) A bar chart representation of the number of lncRNA transcripts differentially upregulated or downregulated after T1-44 treatment in the CT26 (30% change, q<0.05) RNA-seq datasets.

B) CT26 cells were transfected with two different siRNAs targeting E2F1 for 72 h. They were additionally treated with 1 μM T1-44 8 h post-transfection, prior to RT-qPCR analysis to determine the expression of the indicated lncRNA transcripts (labelled with their ENSEMBL transcript name). An immunoblot is also included to display input protein levels of E2F1, and SDMe was used as a marker for T1-44 activity.

C) (i) Schematic representation of the experiment with T1-44 in colon26 tumours (i). Balb/c mice were treated with orally administrated T1-44 at 100 mg/kg for 19 days with respect to vehicle only control; n=7 per group; ii) Absolute tumour growth volume in T1-44 treated (squares) and non-treated (circles) Balb/c mice presented as a mean value (+/−SEM), n=7 iii) scatter plots of absolute tumour volume of individual mice at day 12 (t-test; *p<0.05), n=7; T1-44 treated (on right hand side) and non-treated (on left hand side). iv) Relative body weight representation of T1-44 treated (squares) and non-treated (triangles) Balb/c mice presented as a mean value, n=7; v) survival curves of treated (dotted line) and non-treated mice (solid line—terminates at d16) (Log-rank (Mantel-Cox) test; *p<0.05), n=7;

D) RNA was isolated from colon26 tumours treated with DMSO or T1-44 as indicated, prior to RT-qPCR analysis to determine the expression of the indicated lncRNA transcripts (labelled with their ENSEMBL transcript name).

E) i) Representative examples of immunohistochemical staining of SDMe in colon26 tumours collected from Balb/c mice at 19 days post treatment with 100 mg/kg T1-44, or at day 14 from non-treated controls (see experiment in FIG. 2C). Original magnification: 20×, scale bar, 50 μm; and 63×; scale bar, 16 μm. n=4; ii) As above, but immunohistochemical staining was performed with anti-CD8; iii) As above, but immunohistochemical staining was performed with anti-CD4; iv) As above, but immunohistochemical staining was performed with anti-CD163; v) Results were quantified by ImageJ Fiji software and normalised optical density was presented as a mean+/−SD. Statistical analysis was performed using two-tailed, unpaired Student's t-test with GraphPad Prism 8 software, n=4. Controls are shown in the upper panels, T1-44 shown in the lower panels.

FIG. 3: Immuno-peptidomic analysis of CT26 cells

A) A diagram to indicate the workflow of the immuno-peptidomics platform. Peptides extracted from cell surface MHC class I protein complexes are detected by mass spectrometry and identified by comparison against the in-house lncRNA proteomics database, or a database consisting of all reviewed mouse SwissProt entries. Candidate peptides are then ranked based on their predicted immunogenicity, MHC binding affinity and RNA expression profile, before selection as part of a dendritic cell-based cancer vaccine.

B) CT26 cells were treated for 72 h with 1 μM T1-44 or DMSO (control) prior to immunoprecipitation with antibodies specific to MHC class I alleles and extraction of MHC bound peptides for mass spectrometry analysis. The experiment was performed in biological replicate (rep1, rep2). Indicated in the Venn diagram is the overlap of MHC bound lncRNA derived peptides identified from the qualitative immuno-peptidomics analysis between each treatment and biological replicate. 328 peptides were detected using Peaks software.

C) i) The predicted allele frequency for each MHC allele bound by identified lncRNA-derived peptides from the CT26 immuno-peptidomics analysis is displayed on the bar graph, represented as a percentage of total; ii) Sequence logos demonstrating the amino acid residue conservation in MHC class I bound lncRNA-derived peptides derived from protein coding genes identified from the immuno-peptidomics analysis for each MHC allele

D) The peptide length of each peptide identified as being derived from the murine lncRNA data base is displayed on the graph. 195 peptides were detected in quantitative analysis using Progenesis software.

E) Part of the sequence of the Gm37283, Gm17173, and Gm37494 lncRNA transcripts are displayed (SEQ ID Nos 177, 178 and 179, respectively), with the predicted ORF (shown in grey highlight) giving rise to the identified MHC class I bound peptide (boxed). Amino acid sequences are recited in SEQ ID NOs: 180-187.

F) (i) An example polysome profiling assay performed in CT26 cells treated for 72 h with 1 μM T1-44 or DMSO, indicating total RNA quantity detected in each collected fraction (by absorbance reading at 254 nm). Fractions representing free, unbound RNA (fractions 1-5); 80S ribosomal fractions (fraction 6); and polysome fractions (fractions 7-12) with increasing polysome size are indicated. (ii) Polysome profiling assay for Gm37494 lncRNA giving rise to an MHC class I peptide is displayed from CT26 cells treated with DMSO or 1 μM T1-44 for 72 hours. Data are presented as percentage of total RNA in each fraction; n=3; (iii) Quantitation of polysome profiling assays by calculation of the heavy (fractions 10-12) to light (fractions 6-9) polysome ratios from each of the indicated lncRNA ribosome profiling assays; (iv) An immunoblot is included to demonstrate SDMe levels as a measure of T1-44 activity

FIG. 4: Immuno-peptidomic analysis of HCT116 cells

A) HCT116 cells were treated for 48 h with 1 μM T1-44 or DMSO (control) prior to immunoprecipitation with antibodies specific to MHC class I alleles and extraction of MHC bound lncRNA-derived peptides for mass spectrometry analysis. The experiment was performed in biological replicate (rep1, rep2). Indicated in the Venn diagram is the overlap of MHC bound lncRNA derived peptides identified from the qualitative immuno-peptidomics analysis between each treatment and biological replicate. 55 peptides were identified from the qualitative GENCODE annotated database.

B) (i) The predicted allele frequency for each MHC allele bound by identified lncRNA-derived peptides from the immuno-peptidomics analysis is displayed on the bar graph, represented as a percentage of total. ii) Sequence logos demonstrating the amino acid conservation in MHC class I bound peptides derived from lncRNA genes identified from the immuno-peptidomics analysis for each MHC allele

C) The peptide length of each peptide identified as being derived from the lncRNA GENCODE database (PROGENESIS software analysis) is displayed on the graph. 76 peptides were identified from the quantitative GENCODE database.

D) (i) An example polysome profiling assay performed in HCT116 cells treated for 48 h with 1 μM T1-44 or DMSO, indicating total RNA quantity detected in each collected fraction (by absorbance reading at 254 nm). Fractions representing free, unbound RNA (fractions 1-5); 80S ribosomal fractions (fraction 6); and polysome fractions (fractions 7-12) with increasing polysome size are indicated; (ii) Polysome profiling assays for MALAT1 and AC079135.1 lncRNAs are displayed from HCT116 cells treated with DMSO or 1 μM T1-44 for 48 hours. Data are presented as percentage of total RNA in each fraction; n=3; (iii) Quantitation of polysome profiling assays by calculation of the heavy (fractions 10-12) to light (fractions 6-9) polysome ratios from each of the indicated lncRNA ribosome profiling assays from HCT116 WT and E2F1 Cr cells treated with DMSO or 1 μM T1-44 for 48 hours; (iv) An immunoblot is included to demonstrate SDMe levels as a measure of T1-44 activity.

E) (i) A schematic representation of the pSF-CMV-NEO-COOH-FLAG plasmid that was used as the cloning vector for insertion of predicted ORFs from human lncRNA transcripts found to encode peptides presented on MHC class I. The predicted ORF and a short section of upstream sequence (containing any endogenous Kozak sequence) was ligated into the multiple cloning site (MCS) of the vector, in frame with the C-terminal 3×FLAG tag. (ii) Part of the sequence of the MALAT1 and AC079135.1 lncRNA transcripts is displayed (SEQ ID NOs: 188, 189), with the predicted ORF (shown in grey highlight) giving rise to the identified MHC class I bound peptide (boxed). Amino acid sequences are recited in SEQ ID NOs: 189-193). This ORF was cloned into the pSF-CMV-NEO-COOH-3×FLAG vector upstream of the C-terminal FLAG tag. (iii) HCT116 cells were transfected with 4 μg of the MALAT1 and AC079135.1 ORF-Flag plasmid for 48 h prior to immune fluorescence analysis with anti-Flag antibodies. Cell nuclei were stained with DAPI. (iv) HCT116 cells were transfected with 4 μg of MALAT1 and AC079135.1 ORF-Flag plasmids prior to immunoblot analysis with Flag antibodies.

F) HCT116 WT E2F1 and HCT116 E2F1 Cr cells were transfected with 4 μg of MALAT1 (MAL) or AC079135.1 (AC) ORF-Flag plasmids and 0.5 μg GFP plasmid for 72 h. Cells were also treated with DMSO or 1 μM T1-44 for the last 48 h, prior to immunoblot analysis with the indicated antibodies. SDMe was included to demonstrate activity of the T1-44 compound.

FIG. 5

A) Heat maps of peptide encoding lncRNA transcripts comparing expression in tumour versus normal tissues using TCGA and Cancer Cell Line datasets. Blue heatmap (top)—Expression presented as a mean of all samples [log 2(fpkm+0.001)] dependent on anatomical site of the tumour corresponding to normal tissue; red/green heatmap (bottom left)—representation of tumour/normal ratio [Log 2(Tumour/Normal FPKM ratio)], red (abundant in MALAT1) represents higher expression in normal when green represents the higher expression in tumour tissue; orange heatmap (bottom middle)—expression level in different colorectal cancer cell lines [Log 2(fpkm+0.001)]; light blue (bottom right)—Row Z-score normalised expression level in microsatellite stable vs instable patients.

B) Kaplan Meier curves of overall survival of patients with adrenocortical carcinoma (i), colorectal (ii), and pancreatic cancer (iii) for the expression of several lncRNA genes. Plots were generated using Gepia2 tool. For each analysis patients were divided into two groups: one with high expression (red line) or one with low expression (blue line.)

Characterisation of the 20 selected peptides encoded by murine lncRNAs identified in the immuno-peptidomics experiment on CT26 cells treated with T1-44, with respect to DMSO control. In order from the left, columns characterise: sequence of the peptide, lncRNA gene name, transcript accession ID; peptide length; Net MHCpan score and allele columns show the results from binding affinity prediction analysis; Peptide abundance fold change (T1-44 treated vs DMSO treated) (derived using PROGENESIS software); expression level in CT26 cells (based on our in house RNA-seq and other databases—GENEVESTIGATOR software) (Low—log 2TPM<7.5; Medium—log 2TPM 8.5-11.5; High—log 2TPM>11.5); expression in thymus (based on EXPRESSION ATLAS—www.ebi.ac.uk/gxa/home and GENEVESTIGATOR software)(Low—log 2TPM<7.5; Medium—log 2TPM 8.5-11.5; High—log 2TPM>11.5; below cut-off—no expression); last column represents the results from immunogenicity experiment.

FIG. 6: LncRNA derived MHC class I peptides as cancer vaccines in a colon26 tumour model

A) (i) Schematic representation of the immunogenicity assay used to measure immune responses against MHC class I bound peptides identified as being derived from lncRNAs. Briefly, Balb/c mice were vaccinated at day 0 with 20 peptides (50 μg each) divided into 4 groups (4 mice in each group) with pools of 5 peptides each, and a booster vaccination was given 7 days later. As adjuvants, CD40 antibody and poly:IC were used. As a positive control AH1 peptide (SPSYVYHQF; SEQ ID NO: 156) vaccination was used. Mice were culled 7 days post-boost and their spleens removed to isolate splenocytes for ELispot assay. (ii) Splenocytes from each separate mouse were stimulated with the respective individual peptides (15 μg/ml) that the group had been vaccinated with. Each peptide was tested in duplicate with the indicated peptides and activity was measured in interferon gamma-based ELispot assay. DMSO was used as a negative control. The spots were quantitated on an ELISPOT counter.

B) (i) Schematic representation of the dendritic cell (DC) based vaccine strategy used in a Colon26 tumour challenge experiment. Randomised Balb/c mice were vaccinated with dendritic cells (DCs) pulsed with pools of 15 lncRNA derived peptides at Day 0 and the second dose at day 7. As a control, unpulsed dendritic cells were used; n=6. ii) Absolute tumour growth volume in unpulsed and pulsed DC treated BALB/c mice presented as a mean value (+/−SEM), n=6 iii) scatter plots of absolute tumour volume of individual mice at day 12 (t-test; *p<0.05), n=6; iv) Relative body weight representation of unpulsed and pulsed DCs treated BALB/c mice presented as a mean value, n=7;

C) Model diagram to indicate regulation of lncRNA-derived antigen presentation by the E2F1-PRMT5 axis. It is proposed that PRMT5-directed methylation of E2F1 influences its transcriptional activity on a number of genes, including the expression of many lncRNAs, which are subsequently translated into polypeptides that can be processed to generate peptide epitopes for presentation on MHC class I protein complexes. Pharmacological manipulation of PRMT5 activity with compound T1-44 results in altered expression of several lncRNA transcripts encoding immunogenic peptides. We propose that subsequent presentation of these immunogenic peptides by MHC class I complexes contributes to the increased immune cell infiltration of the tumour micro-environment (TME) observed.

### SUMMARY OF THE INVENTION

According to a first aspect of the invention there is provided a peptide derived from a PRMT5-E2F1 axis regulated long non-coding RNA gene or a derivative thereof.

According to a second aspect of the invention there is provided a nucleic acid sequence encoding a peptide according to the first aspect of the invention.

According to a third aspect of the invention there is provided a vector comprising a nucleic acid of the second aspect of the invention. Suitably, the vector is an expression vector capable of expressing the nucleic acid of the second aspect of the invention. Expressing the nucleic acid of the invention also includes producing the peptide encoded by the sequence.

According to a fourth aspect of the invention there is provided a host cell comprising the nucleic acid of the second aspect of the invention or the vector (e.g. expression vector) of the third aspect of the invention.

According to a fifth aspect of the invention there is provided a vaccine comprising at least one peptide according to the first aspect of the invention or the nucleic acid of the second aspect of the invention or the vector of the third aspect of the invention.

According to a sixth aspect of the invention there is provided a method for making a tumour specific vaccine, comprising identifying peptides expressed by a tumour that are encoded by a PRMT5-E2F1 axis regulated long non-coding RNA gene; and incorporating one or more of said peptides or a nucleic acid encoding said one or more peptide(s) into a vaccine. Optionally, said peptides are also tested to confirm that they are immunogenic or bind to MHC class 1 molecules.

According to a seventh aspect of the invention there is provided a pharmaceutical composition comprising the peptide of the first aspect of the invention, the nucleic acid of the second aspect of the invention, or the vaccine of the fifth aspect of the invention and a pharmaceutically acceptable excipient.

According to an eighth aspect of the invention there is provided the pharmaceutical composition of the seventh aspect of the invention or the peptide of the first aspect of the invention, or the nucleic acid of the second aspect of the invention, or the vaccine of the fifth aspect of the invention for use in therapy. Suitably, the therapy is the treatment of cancer. In a particular embodiment, the treatment of cancer involves administration of the pharmaceutical composition of the seventh aspect of the invention or the peptide of the first aspect of the invention, or the nucleic acid of the second aspect of the invention, or the vaccine of the fifth aspect of the invention in combination with a PRMT5 inhibitor.

According to a ninth aspect of the invention there is provided a method for selecting peptides for inclusion in a tumour vaccine, comprising contacting a tumour cell sample with a PRMT5 inhibitor and determining the expression level of one or more lncRNA gene encoded peptides by the contacted (treated) cells and selecting one or more peptides that are dysregulated and immunogenic for inclusion in a tumour vaccine. Suitably, the tumour cell sample is taken from a patient with a cancer/tumour. Suitably the peptide is one that is upregulated in the tumour cell.

Thus, in a variation of the ninth aspect of the invention there is provides a method for selecting peptides for inclusion in a tumour vaccine, comprising contacting cells of a tumour type with a PRMT5 inhibitor and determining the expression level of one or more lncRNA gene encoded peptides by the contacted cells and selecting one or more peptides that are upregulated for inclusion in a tumour vaccine, optionally prior to selection the peptides are also tested to determine or predict whether they are immunogenic and one or more immunogenic peptides are selected for inclusion in a tumour vaccine.

According to a tenth aspect of the invention there is provided a method for making a vaccine, comprising selecting the peptides for inclusion into the vaccine according to the ninth aspect of the invention and generating a vaccine capable of presenting said peptides.

According to an eleventh aspect of the invention there is provided a PRMT5 inhibitor for use in the treatment of cancer by stimulating an immune response.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following:

### DETAILED DESCRIPTION OF THE INVENTION

The disclosed methods may be understood more readily by reference to the following detailed description which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

The methods of the present disclosure will employ, unless otherwise indicated, conventional techniques of molecular biology (including recombinant techniques), microbiology, cell biology, and biochemistry, which are within the skill of the art. Exemplary techniques are explained fully in the literature, such as, “Molecular Cloning: A Laboratory Manual”, 2nd edition (Sambrook et al., Molecular Cloning: A Laboratory Manual, New York: Cold Spring Harbor Press, 1989); “Current Protocols in Molecular Biology” (F. M. Ausubel et al., eds., Current Protocols of Molecular Biology, John Wiley and Sons (1987); and “PCR: The Polymerase Chain Reaction”, (Mullis et al., eds., Birhsuser, Boston, 1994).

Unless indicated otherwise, each gene name used herein corresponds to the Official Symbol assigned to the gene and provided by Entrez Gene (URL: www.ncbi.nlm.nih.gov/sites/entrez) as of the filing date of this application.

### Definitions

Reference to a particular numerical value includes at least that particular value, unless the context clearly dictates otherwise. When a range of values is expressed, another embodiment includes from the one particular value and/or to the other particular value. Further, reference to values stated in ranges include each and every value within that range. All ranges are inclusive and combinable.

It is to be appreciated that certain features of the disclosed methods, which are for clarity, described herein in the context of separate embodiments, may also be provided in combination in a single embodiment. Conversely, various features of the disclosed methods that are, for brevity, described in the context of a single embodiments, may also be provided separately or in any sub-combination.

The articles “a,” “an,” and “the” are used herein to refer to one or to more than one (i.e. to at least one) of the grammatical object of the article.

The use of the alternative (e.g., “or”) should be understood to mean either one, both, or any combination thereof of the alternatives.

The term “and/or” should be understood to mean either one, or both of the alternatives.

When values are expressed as approximations, by use of the antecedent “about”, it will be understood that the particular value forms another embodiment.

As used herein and unless stated otherwise, it is to be understood that the term “about” is used synonymously with the term “approximately”. Illustratively and unless stated otherwise, the use of the term “about” indicates values slightly outside the cited criteria values, for example ±15%, ±10%±8%, ±5% or conveniently ±2%. Such values are thus encompassed by the scope of the claims reciting the terms “about” or approximately”.

As used herein, the term “in vitro” means performed or taking place in a test tube, culture dish, or elsewhere outside a living organism. The term also includes ex vivo because the analysis takes place outside an organism.

As used herein, the term “isolated” means material that is substantially or essentially free from components that normally accompany it in its native state. In the context of isolated from a subject it can mean is removed from the subject. In particular embodiments, the term “obtained” or “derived” is used synonymously with isolated.

A “subject,” “individual,” or “patient” as used herein, includes any animal that can be tested using the present invention. Suitable subjects include laboratory animals (such as mouse, rat, rabbit, or guinea pig), farm animals (such as horses, cows, sheep, pigs), and domestic animals or pets (such as a cat or dog). In particular embodiments, the subject is a mammal. In certain embodiments, the subject is a non-human primate and, in a particular embodiment, the subject is a human.

### Peptides of the Invention

The present invention is based in part on the recognition that tumour cells subjected to inhibition of PRMT5 results in an increased expression of various peptides by the tumour cells, including on the cell surface, which the inventors have determined are coded for by long non-coding (Inc) RNA genes. Such peptides are regulated by PRMT5-E2F1 axis. A bioinformatic analysis of the differentially expressed lncRNAs found that the majority of these were associated with or possessed an E2F binding site implying that these are under transcriptional control of E2F1.

The inventors have discovered that many of these peptides are immunogenic, capable of binding to MHC-class I molecules, and elicit a host immune response, which resulted in a slowing of tumour cell growth. These peptides are thus a new source of tumour associated antigens that can be utilised in vaccine design.

One aspect of the invention also relates to a PRMT5 inhibitor for use in treating cancer by stimulating a host immune response against the tumour cells. This new mechanism of targeting tumour cells offers up new clinical uses, including the ability to treat cancers that have acquired resistance to the direct cancer killing effect of PRMT5 inhibition. Furthermore, dendritic cells exposed to these peptides were capable of slowing down tumour cell growth demonstrating that these peptides can serve as agents for stimulating a host immune response and thus suitable for use in a vaccine. The peptides of the invention can thus be used in therapy, particularly in the treatment of cancer.

Much of the human genome is made up of non-classical genes, composed of different structural and regulatory elements and includes genes for example encoding microRNAs and long non-coding (Inc) RNAs (Gebert and MacRae, 2019; Statello et al., 2021). LncRNA genes (referred to herein as lncRNAs or Incgenes) are a major source of transcription in mammalian cells, typically encoding transcripts with lengths of over 200 nucleotides. However, whilst a small number of lncRNA transcripts have been shown to be processed in the same way as mRNA, and in rare cases suggested to perform biological roles, most of them were believed to exist as untranslated RNAs (Statello et al., 2021).

According to the first aspect of the invention there is provided a peptide derived from a PRMT5-E2F1 axis regulated long non-coding RNA gene or a derivative thereof.

Suitably, the peptide is immunogenic. Suitably, the peptide is an MHC-class I associated peptide or a derivative thereof. Suitably, the peptide is presented by a human leukocyte antigen (HLA) class I molecule. Suitably, the peptide is isolated.

The term “isolated” as used herein generally means a biological component (such as a nucleic acid molecule, protein or peptide) that has been substantially separated or purified away from other biological components in the cell of the organism in which the component naturally occurs, i.e., other chromosomal and extrachromosomal DNA and RNA, and proteins; and/or is a shorter component of the natural molecule. Nucleic acids and proteins that have been “isolated” include nucleic acids and proteins purified by standard purification methods. The term also embraces nucleic acids and proteins prepared by recombinant expression in a host cell as well as chemically synthesized nucleic acids, proteins and peptides.

Suitably the lncRNA transcript has a predicted E21F binding site.

Suitably the peptide has an amino acid sequence that is encoded by part of an lncRNA transcript that also has a predicted E21F binding site. The E21F binding site is a DNA sequence in the promoter of the gene that binds E2F1. E2F1 is a transcription factor; once bound to the DNA sequence, E2F1 can then drive transcription of the gene. The canonical E2F1 binding site sequence is TTTSSCGGC where S is guanine or cytosine.

Suitably, the peptide is one disclosed in Table 1, or a derivative of a peptide disclosed therein.

TABLE 1

| | SEQ ID |
|---|---|
| Sequence | NO: |
| ASEPRPFWGY | 1 |
| GSDRGLYLEY | 2 |
| ALIKHVANA | 3 |
| IMGEFRTEV | 4 |
| MVAESPPRV | 5 |
| RLATHIDGA | 6 |
| RLLQETHQA | 7 |
| AMVAESPPRV | 8 |
| LTEKELLNY | 9 |
| MESHSVAQA | 10 |
| SEVDSVRDRLP | 11 |
| TEIEAIHF | 12 |
| AEEERRPPA | 13 |
| AEKPPGSVA | 14 |
| AESAIPVRT | 15 |
| MEATHSTLP | 16 |
| MESHFVAQA | 17 |
| HRARSIFIF | 18 |
| THAKSFLVF | 19 |
| EETYFHLF | 20 |
| MEERVVRIA | 21 |
| AEAGELLEPGR | 22 |
| GMQGPPPGQVP | 23 |
| EAGAGAVEGRVA | 24 |
| LCDSLPRY | 25 |
| VSDHPALLY | 26 |
| LTEKELLNY | 27 |
| GSDRGLYLEY | 28 |
| ASEPRPFWGY | 29 |
| SISSPTLLLY | 30 |
| MSSGMTKDSLY | 31 |
| VMGLLDLSERY | 32 |
| ASDYGSEDSRY | 33 |
| ATEHDKNSTNIY | 34 |
| YSETSLSNGGKY | 35 |
| RLATHIDGA | 36 |
| HLLDVPVVL | 37 |
| RLLQETHQA | 38 |
| HLIQEEIIL | 39 |
| ALAEVFHQL | 40 |
| IMGEFRTEV | 41 |
| GVVSLFVGL | 42 |
| HLLQKELIL | 43 |
| VMLESPPKGV | 44 |
| KILDLVSGAL | 45 |
| EETYFHLF | 46 |
| EHIILNTL | 47 |
| MESPSVRP | 48 |
| AEKPPGSVA | 49 |
| AEAGRSLEA | 50 |
| RESKHIHAP | 51 |
| EEHQKMNKP | 52 |
| AEARGSPEV | 53 |
| MESHSVAQA | 54 |
| SEEPLRLAA | 55 |
| AEVGRSPEV | 56 |
| AERFSTLTA | 57 |
| MEERVVRIA | 58 |
| RESRVTPPP | 59 |
| AETGSHYVA | 60 |
| TEMWDLRAP | 61 |
| MEFHSVAQA | 62 |
| QEDPSSHLIIA | 63 |
| KIDSESIIL | 64 |
| FQPNNFQKY | 65 |
| ISHDSLVLL | 66 |
| SFKSNGLSM | 67 |
| KTQQAHTA | 68 |
| THYDSIYK | 69 |
| ISDFFCCC | 70 |
| ATRDLHTA | 71 |
| KNRHLLEL | 72 |
| WLTRPVGV | 73 |
| HHHHHPSSS | 74 |
| PCVETPSQR | 75 |
| HHHHHHPSSS | 76 |
| LHLLLFLVIV | 77 |
| LTKSTNILYII | 78 |
| QASHVPGAAAG | 79 |
| SLDVGGPLRY | 80 |
| WILMESCINF | 81 |
| LTEKELLNY | 82 |
| TEIEAIHF | 83 |
| ESDVSSALSY | 84 |
| CWIKWENAY | 85 |
| ALYEKDNTYL | 86 |
| TFSPCHFFLT | 87 |
| ATRDLHTA | 88 |
| LSSYHLAHVI | 89 |
| TFFFNLEEF | 90 |
| QNTREIFTY | 91 |
| EETYFHLF | 92 |
| LMEHIHKL | 93 |
| RESRVTPPP | 94 |
| QEDPSSHLIIA | 95 |
| EETGTAISP | 96 |
| AEERWEVVL | 97 |
| AEAEGSLGP | 98 |
| AEAGRSPEV | 99 |
| ASEPRPFWGY | 100 |
| ILMEHIHKL | 101 |
| DTIEVAEV | 102 |
| ITNKYQLVF | 103 |
| IRRENIRFL | 104 |
| HLIQEEIIL | 105 |
| AEKPPGSVA | 106 |
| ILMEHIHKLKA | 107 |
| STDGIGKSY | 108 |
| TLCDLYETL | 109 |
| MEERVVRIA | 110 |
| ASGPPVTL | 111 |
| AEVGRSLEA | 112 |
| KLLDPRIYYI | 113 |
| KTQQAHTA | 114 |
| MEFHSVAQA | 115 |
| ISHDSLVLL | 116 |
| MILLKLTSV | 117 |
| ELDLKATQA | 118 |
| KRFESWRVL | 119 |
| DTSWPLSL | 120 |
| MERRDIFTV | 121 |
| SEEPLRLAA | 122 |
| EEHQKMNKP | 123 |
| RESKHIHAP | 124 |
| LSRTRKGP | 125 |
| AEVGRSPEV | 126 |
| LSLSLSLQFS | 127 |
| LLDPRIYYI | 128 |
| AEERNLYV | 129 |
| MESHSVAQA | 130 |
| DLLLKETQL | 131 |
| YEINAHKY | 132 |
| EAERGESLDS | 133 |
| TEAEAHLQA | 134 |
| MQIFVKTLT | 135 |
| AETGSHYVA | 136 |
| PIVNTFNVFCNS | 137 |
| PEIKLHTY | 138 |
| EEMPLNVA | 139 |
| NLDPAVHEV | 140 |
| HCIQDNIYY | 141 |
| QSLSARGSAP | 142 |
| ETFRSCSFV | 143 |
| KNVFSQEL | 144 |
| IMGEFRTEV | 145 |
| IICMSFVNF | 146 |
| NSISKKKKN | 147 |
| ATEHDKNSTNIY | 148 |
| KNRHLLEL | 149 |
| HLLDVPVVL | 150 |
| HHHHHHPSSS | 151 |
| HHHHHPSSS | 152 |
| HLLQKELIL | 153 |
| AEAGGLLEP | 154 |
| HLLDVPVVL | 155 |

The peptide may be of any length from 5 amino acid upwards. Typically, the peptide is between 6 and 30 amino acids long, inclusive, such as between 8 and 20 amino acids long, inclusive. The immunogenic peptides presented by MHC1 identified in the Examples had an average length of 9 amino acids. In a particular embodiment, the immunogenic peptide is 8, 9 or 10 amino acids long.

A peptide of the first aspect may be referred to herein as a peptide of the invention or lncRNA gene peptide of the invention.

A “derivative” of one of the peptides refers to a mutant or variant peptide, which may contain amino acid deletions, additions or substitutions, subject to the requirement to maintain the immunogenicity of the peptide. Thus, conservative amino acid substitutions may be made substantially without altering the nature of the peptide, as may truncations from the 5′ or 3′ ends. Deletions and substitutions may moreover be made to the peptides of the invention. Substitutional, deletional or insertional variants of the peptides can be prepared by recombinant methods and screened for immuno-cross reactivity with the native peptide. A derivative also encompassed fusion proteins wherein one part of the fusion protein is the peptide of the invention and the other some other peptide sequence. By “derived from” includes the situation where the peptide is a part of the polypeptide that is expressed from the lncRNA. Thus, by way of illustration, if the lncRNA gene encodes a polypeptide of 80 amino acids in length, a peptide derived therefrom may be a smaller part thereof, such as a peptide of 8 or 9 or more amino acids, but fewer that the 80.

Thus, the peptide derived from a PRMT5-E2F1 axis regulated long non-coding RNA gene may be the entire translated polypeptide or a part thereof, i.e. a peptide. Suitably the peptide is immunogenic. Suitably, the peptide is capable of being presented on MHC class 1 molecules, for example, as an antigen-MHC-class 1 complex.

In particular embodiment the derivative of the peptide comprises at least 80%, such as at least 85%, at least 90%, or at least 95% amino acid sequence identity with the peptide. With respect to the parent lncRNA gene encoded peptide, such derivative peptide may be referred to as a modified variant of the parent lncRNA gene encoded peptide.

### Nucleic Acids and Host Cells

According to a second aspect of the invention there is provided a nucleic acid sequence encoding a peptide according to the first aspect of the invention.

Suitably the nucleic acid is DNA, cDNA, PNA, RNA or combinations thereof.

According to a third aspect of the invention there is provided an vector comprising the nucleic acid of the second aspect of the invention. Any vector, such as a plasmid, a cosmid, a viral vector may be used. The vector can be used to amplify the number of copies of the insert sequence using standard approaches of, for example bacterial transformation, cell culture and vector purification. The vector may also facilitate the expression of the encoded protein.

In a particular embodiment the vector is an expression vector and capable of expressing the nucleic acid of the second aspect of the invention. Expressing the nucleic acid of the second aspect of the invention also includes producing the peptide encoded by the sequence.

According to a fourth aspect of the invention there is provided a host cell comprising the nucleic acid of the second aspect of the invention or the vector of the third aspect of the invention.

Any suitable host cell, e.g. prokaryotic or eukaryotic, can be used, in particular embodiments the host cell is selected from the group consisting of a bacterial, fungal (including yeast), and mammalian cell.

### Vaccines

Cancer vaccines generally relate to one of the following categories: protein antigen/adjuvant vaccines, DNA vaccines, viral vector-based vaccines, tumour cell vaccines, and dendritic cell vaccines. Any of these types of vaccine can be employed in the present invention.

According to a fifth aspect of the invention there is provided a vaccine comprising at least one peptide according to the first aspect of the invention or the nucleic acid of the second aspect of the invention or the vector of the third aspect of the invention.

Vaccine technologies are well advanced and any vaccine platform that is capable of presenting the peptides of the invention to the immune cells of the body can be employed. Suitably, the vaccine is a protein antigen/adjuvant vaccines, peptide vaccine, RNA vaccine, DNA vaccine, vector vaccine or dendritic cell vaccine. Suitably, the vector vaccine is a viral vector vaccine.

Peptide vaccines can be used wherein the antigen peptide of choice is include in a vaccine and presented on antigen presenting cells to trigger the immune response. Peptide vaccines are often co-administered with an adjuvant to prime a local immune response. The peptide needs to stimulate CD8+ and/or CD4+ specific T cells and so the majority of peptide vaccines use peptides of around 8-30 amino acids in length that contain nested CD8+ T cell and/or CD4+ T cell epitopes that comprise an immunogenic portion thereof. The immunogenic portion is the peptide that is bound by MHC1, such immunogenic peptide will typically be 8, 9 or 10 amino acids long, 9 being most usual. Suitably, multi-peptide vaccines are used so that many antigens can be targeted at the same time to generate a polyclonal antigen T cell response, enhance the immune response and mitigate for any antigen loss on the tumour cell.

The advantages of peptide vaccines is that they can be chemically synthesised and manufactured at large scale and at reduced costs compared to other cancer treatments.

The administration of free adjuvant with peptide antigens in a cancer vaccine can result in their dissociation following injection. One option to address this is to link the adjuvant and peptide antigen together, e.g. by direct conjugation. Peptides can be linked to hydrophobic carriers such as lipids, fatty acids and TLR agonists for more efficient delivery to antigen presenting cells.

In particular embodiment of the invention, one or more peptides of the invention is conjugated to an adjuvant.

Nucleic acid based vaccines are designed to enter the cells and transcribe, translate and process the antigen epitopes (e.g. peptide(s) of the invention) so that they can be presented to MHC and elicit a host immune response.

DNA vaccines are simple to design, relatively cheap to produce and have reasonable stability (at 2-8° C.) and solubility. Plasmid DNA vaccines can be designed to act as both antigen and adjuvant, unmethylated DNA containing a CG-rich region can also act as an adjuvant. Plasmid DNAs can be designed so that they are taken up into antigen presenting cells, where they are transcribed, translated and processed with epitopes (e.g. immunogenic peptides of the invention) presented on the cell surface in combination with MHC.

RNA vaccines have the advantage that they are not incorporated into the host cell genome and thus avoid the safety concerns associated with such integration. They are easy to design, can encode multiple epitopes and being single stranded they also stimulate TLP7 and 8 and so have adjuvant function. They also only need to be delivered to the cytoplasm for translation into protein, unlike DNA that needs to be delivered to the nucleus for transcription. However, they are much more susceptible to nuclease (e.g. RNAse) degradation and so are more labile than DNA vectors.

Viral vectors have been designed and created from numerous viruses. The most commonly used viral vectors are derived from adenoviruses, poxviruses and alphaviruses. The majority are replication defective or attenuated versions of these. A disadvantage of viral vectors is that they are recognised as foreign by the immune system and so repeat immunisation with the same or similar vector can result in the immune system neutralising these vectors thus diminishing their effect and preventing effective repeat administration.

Suitably, the viral vector is derived from a virus such as adenovirus, adeno-associated virus (AAV), herpesvirus, poxvirus, alpha virus (such as Semliki Forest virus, Sindbis virus, or Venezuelan equine encephalitis virus), arenaviruses (like lassa virus, machupovirus, or junin virus), measles virus, vaccinia virus, retrovirus (including lentivirus), or influenza virus.

Some are double stranded DNA viruses (like adenovirus); some are single stranded RNA viruses (like flavivirus and alphaviruses) which require reverse transcription of the genome into DNA and then subsequent expression of the encoded polypeptides.

The ChAdOx1 vector is an example of a suitable adenovirus vector. More recently this vector has been used as a platform for the COVID-19 vaccine known as ChAdOx1 nCoV-19. Other suitable adenovirus vectors that can be employed in the present invention include, but are not limited to: Ad5-S-nb2 and Ad26-S.

For a review of viral vectors for therapeutic use, including vaccine use, please consult Lundstrom (2020). Table 2 therein provides a list of preclinical and clinical cancer vaccines studies as of 2020 and identifies the particular vaccine vector being employed.

In particular embodiments, the vaccine vector for use in the present invention is selected from the group consisting of: ChAdOx1, Ad5-S-nb2, Ad26-S, Ad5/35, SFV (semliki forest virus), AAV (adeno associated virus), KUN, VSVAG, HSV-1 T-VEC and VEE (Venezuelan equine encephalitis).

Cell based vaccine can also be used. Dendritic cells are particularly suitable for cancer vaccines given their ability to uptake and present tumour-associated antigens (TAA) through a variety of mechanisms, priming effector responses against the tumour cell. Besides direct antigen presentation, the DC can move between lymphoid and non-lymphoid tissue and modulate cytokine and chemokine gradient s to control inflammation and lymphocyte homing, all of which are important for systemic and long-lasting anti-tumour effects. Different DC populations can be used, but mounting evidence suggests that conventional type 1 DCs (cDC1s) play an integral role in tumour immunity and represent a promising alternate cell type for vaccination purposes.

Patient derived DCs can be exposed to the antigen peptides of the invention ex vivo using established protocols and reimplanted into the patient. The cells may be allogeneic or autologous. Cells that have been correctly pulsed with the peptides should then be capable of antigen presentation, in vivo

When using a dendritic cell vaccine approach, the dendritic cells can be exposed to multiple immunogenic peptides. The vaccine can therefore be a mixture of the immunogenic peptides of the invention.

One approach, particularly suited to viral vaccines is to include multiple immunogenic lncRNA gene derived peptides into a chimeric polypeptide comprising these peptides in series and presented this as part of a vaccine. Thus, in particular embodiments, the vaccine comprises 1, 2, 3, 4, 5, 6, 7, 8, 9, 10, 11, 12, 13, 14, 15, 20, 25, 30, 35, 40 or more peptides of the invention. Optionally, particularly when involving multiple peptides, such peptides are provided within a chimeric polypeptide comprising the multiple peptides. These peptides may be in series and may be separated by one or more spacer amino acids, and/or protease cleavage sites.

Suitably, the vaccine comprises at least 5 peptides, at least 10 peptides or at least 15 peptides of the invention.

According to a sixth aspect of the invention there is provided a method for making a tumour specific vaccine, comprising identifying immunogenic peptides expressed by a tumour that are encoded by a PRMT5-E2F1 axis regulated long non-coding RNA gene; and incorporating one or more of said peptides or a nucleic acid encoding said one or more peptide(s) into a vaccine.

lncRNA expression is specific to the type of tumour, therefore the vaccine will work preferentially on the tumour that express the lncRNAs from which the peptides were derived.

### Vaccine Adjuvants

In order to enhance the adaptive immune response, vaccines can be administered with an adjuvant which can aid in attracting immune cells to the injection site and promote cell mediated trafficking of antigen to draining lymph nodes and for triggering antigen presenting cells.

Numerous vaccine adjuvants have been developed and tested over the years. Including, water in oil emulsions such as Montanide ISA-51 or Montanide ISA-720; pathogen associated molecular pattern molecules (PAMPs); Toll-like receptor agonists such as polyinosinic-polycytidylic acid with polylysine and carboxymethylcellulose (Pol-ICLC a TLR3 agonist, monolphosphoryl lipid A (MPLA) a TLR4 agonist, imiquimod a TLR7 agonist, resiquimod a TLR7 and TLR8 agonist, CpG oligodeoxynucleotide (CpG ODN) a TLR9 agonist; CD40 agonists; stimulator of interferon genes protein (STING) agonists; cytokines, such as IL-2, IL12, interferon gamma, and granulocyte-macrophage colony stimulating factor (GM-CSF) (for review see Paston et al. 2021.

The majority of cancer vaccines are currently using TLP agonists as adjuvants.

Suitably, peptide vaccines are directly fused to an adjuvant. Peptides can be linked to hydrophobic carriers such as lipids, fatty acids and TLR agonists for more efficient delivery to antigen presenting cells.

In particular embodiments, the vaccines of the present invention are administered with a suitable adjuvant, such as one disclosed above.

The person of skill in the art can select the appropriate adjuvant for use with the selected vaccine.

### Vaccine Delivery Systems

The delivery of nucleic acid-based vaccines into antigen presenting cells (APC) can be enhanced using electroporation, wherein small electrical pulses induce the formation of transient pores in the cell membrane through which the nucleic acid can more easily pass. Due to causing some local tissue damage it stimulates proinflammatory cytokines into the vicinity and so also has an adjuvant effect.

Various nanoparticle based delivery systems, such as polymeric nanoparticles, liposomes, micelles, carbon nanotubes, gold nanoparticles, mesoporous silica nanoparticles, and virus nanoparticles, have also been used for vaccine delivery. Liposomes and lipid-based nanoparticle formulations, such as DOTMA, DOTE, DOTAP, cholesterol, Lipolex, are particularly popular.

The antigen for stimulating an immune response can also be delivered as part of a self-assembling peptide, these systems have certain advantages over liposomes or nanoparticles including, high drug loading, biodegradability and smaller size. Cell penetrating peptide (CPP) based gene vectors, such as glycosaminoglycab (GAG)-binding enhanced transduction (GT) delivery system is one such example.

### Combination

Suitably the vaccines of the invention are administered in combination with another therapeutic agent. Cancer vaccines have the potential to induce potent immune responses but tumour cells have a variety of immune evasion mechanisms that interfere with the function of, and recognition by, T cells. Immune checkpoint inhibitors are cell surface receptors that regulate the immune response. In the TME the expression of checkpoint receptors can suppress T cell activation and thus evade the immune response. The cytotoxic T lymphocyte protein 4 (CTLA4) and programmed cell death protein 1 (PD-1) are the best characterised checkpoint receptors. Antibodies that specifically block the CTLA-4 or PD-1/PD-L1 pathway have the potential to the T-cell immune suppression enabling successful recognition of the tumour antigens and killing of the tumour cells.

Checkpoint inhibitor molecules are thus particularly useful for combination with vaccines. The tumour microenvironment (TME) can lead to immunosuppression of the. The use of a checkpoint inhibitor can relieve this TME driven immunosuppression to maximise the vaccine efficacy.

Many cancer vaccines currently in clinical trials are combined with a checkpoint inhibitor such as CTLA-4, PD-1 or PDL-1 inhibitor. Exemplary checkpoint inhibitor molecules for use in combination with a vaccine of the invention include those which block CTLA-4, such as ipilimumab, those which blocks which block PD-1, such as pembrolizumab or nivolumab, and those which block PDL-1, such as atezolimumab or durvalumab.

### Pharmaceutical Compositions

According to a seventh aspect of the invention there is provided a pharmaceutical composition comprising the peptide of the first aspect of the invention, the nucleic acid of the second aspect of the invention, or the vaccine of the fifth aspect of the invention and a pharmaceutically acceptable excipient.

In this context, the peptide of the first aspect of the invention, the nucleic acid of the second aspect of the invention, or the vaccine of the fifth aspect of the invention may be referred to as “the agent”.

The term “pharmaceutically-acceptable excipient” as used herein means one or more compatible solid or liquid fillers, diluents or encapsulating substances that are suitable for administration into a human. The term “excipient” denotes an organic or inorganic ingredient, natural or synthetic, with which the active ingredient is combined to facilitate the application. Types of suitable excipient are salts, buffering agents, wetting agents, emulsifiers, preservatives, compatible carriers, diluents, carriers, vehicles, supplementary immune potentiating agents such as adjuvants and cytokines that are well known in the art and are available from commercial sources for use in pharmaceutical preparations (see, e.g. Remington: The Science and Practice of Pharmacy with Facts and Comparisons: Drugfacts Plus, 20th Ed. Mack Publishing; Kibbe et al., (2000) Handbook of Pharmaceutical Excipients, 3rd Ed., Pharmaceutical Press; and Ansel et al., (2004) Pharmaceutical Dosage Forms and Drug Delivery Systems, 7th Ed., Lippencott Williams and Wilkins). Optionally, the pharmaceutical compositions contain one or more other therapeutic agents or compounds. Suitable pharmaceutically acceptable excipients are relatively inert and can facilitate, for example, stabilisation, administration, processing or delivery of the active compound/agent into preparations that are optimised for delivery to the body, and preferably directly to the site of action.

The pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use.

When administered, the agent is administered in pharmaceutically acceptable preparations/compositions.

Administration may be enteral (e.g. oral), i.e., substance is given via the gastrointestinal tract, or parenteral, i.e., substance is given by other routes than the digestive tract such as by injection. Large biologic molecules or nucleic acid molecules (such as certain vaccines) are typically administered parenterally by injection.

Pharmaceutical compositions for parenteral administration (e.g. by injection), include aqueous or non-aqueous, isotonic, pyrogen-free, sterile liquids (e.g. solutions, suspensions), in which the active ingredient/agent is dissolved, suspended, or otherwise provided (e.g. in a liposome or other microparticulate). Such liquids may additionally contain one or more pharmaceutically acceptable carriers, such as anti-oxidants, buffers, stabilisers, preservatives, suspending agents, and solutes that render the formulation isotonic with the blood (or other relevant bodily fluid) of the intended patient. In particular embodiments, the composition may be lyophilised to provide a powdered form that is ready for reconstitution as and when needed. When reconstituted from lyophilised powder the aqueous liquid may be further diluted prior to administration. For example, diluted into an infusion bag containing 0.9% sodium chloride injection, USP, or equivalent, to achieve the desired dose for administration. In particular embodiments, such administration can be via intravenous infusion using an intravenous (IV) apparatus.

Suitably, the agent is formulated in accordance with routine procedures as pharmaceutical compositions adapted for intravenous administration to human beings. Typically, the active agent for IV administration is in solution, e.g. in sterile isotonic aqueous buffer. Where necessary, the compositions can also include a solubilizing agent. Compositions for IV administration can optionally include a local anaesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule. Where the agent is to be administered by infusion, it can be dispensed, for example, with an infusion bottle containing sterile pharmaceutical grade water or saline. Where the agent is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example, prepared by conventional means with pharmaceutically acceptable excipients such as binding agents (e.g., pre-gelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavouring, colouring and sweetening agents as appropriate. Oral compositions can include standard vehicles such as mannitol, lactose, starch, magnesium stearate, sodium saccharine, cellulose, magnesium carbonate, and the like.

Compositions for use in accordance with the present invention can be formulated in conventional manner using one or more physiologically acceptable excipients. Thus, the agent and optionally another therapeutic or prophylactic agent and their physiologically acceptable salts and solvates can be formulated into pharmaceutical compositions for administration by inhalation or insufflation (either through the mouth or the nose) or oral, parenteral or mucosal (such as buccal, vaginal, rectal, sublingual) administration. In a particular embodiment, local or systemic parenteral administration is used.

The pharmaceutical compositions for use the treatment methods of the invention are for administration in an effective amount. An “effective amount” is the amount of a composition that alone, or together with further doses, produces the desired response.

Suitably, the agent can be administered as a pharmaceutical composition in which the pharmaceutical composition comprises between 0.1-1 mg, 1-10 mg, 10-50 mg, 50-100 mg, 100-500 mg, or 500 mg to 5 g of the agent.

The preparation of a suitable pharmaceutical composition of the drug and the dosage to administer to a subject is within the capabilities of a person of skill in the art.

### Medical Uses

According to an eighth aspect of the invention there is provided the pharmaceutical composition of the seventh aspect of the invention or the peptide of the first aspect of the invention, or the nucleic acid of the second aspect of the invention, or the vaccine of the fifth aspect of the invention for use in therapy.

According to a variation of this eighth aspect of the invention there is provided a method of treating cancer in a subject suffering from cancer, the method comprising administering to the subject therapeutically effective amount of a pharmaceutical composition of the seventh aspect of the invention or a peptide of the first aspect of the invention, or a nucleic acid of the second aspect of the invention, or a vaccine of the fifth aspect of the invention.

According to a variation of this eighth aspect of the invention there is provided a peptide of the first aspect of the invention, or a nucleic acid of the second aspect of the invention, or a vaccine of the fifth aspect of the invention for use in the manufacture of a medicament for therapy, such as for treating cancer.

In a particular embodiment, the therapy is the treatment of cancer. In a particular embodiment, the treatment of cancer involves administration of the pharmaceutical composition of the seventh aspect of the invention or the peptide of the first aspect of the invention, or the nucleic acid of the second aspect of the invention, or the vaccine of the fifth aspect of the invention in combination with a PRMT5 inhibitor.

Any therapeutic agent capable of inhibiting PRMT5 can be employed in this combination aspect of the invention. In particular embodiments, the PRMT5 inhibitor can be selected from the group consisting of: an antibody, an siRNA, an antisense oligonucleotide (ASO) or a small molecule compound.

The PRMT5 inhibitor for use in this aspect of the invention may be formulated as a pharmaceutical composition as described above for “the agent”. The dosage, route of administration and indeed treatment regime can be determined by the person of sill in the art. PRNT5 inhibitors are further described below.

Suitably, the PRMT5 inhibitor is a small molecule compound selected from the group consisting of: GSK3326595 (pemrametostat), PF-6939999, JVNJ-64619178 (onametostat), LLY-283 and PRT543.

Components of a combination treatment may be administered in combination or conjunction with each other. A combination treatment may be in the form of a combined preparation, for example a combined preparation of an isolated peptide or vaccine of the invention and PRMT5 inhibitor. A combination may comprise separate formulations of each of the components, for example, separate formulations of vaccine and PRMT5 inhibitor.

Components in a combination (e.g. separate formulations) may be administered sequentially, separately and/or simultaneously as described herein in relation to the combination treatment methods. Thus, for example, a vaccine of the invention may be administered separately, sequentially or simultaneously with a PRMT5 inhibitor. In one embodiment the components are administered simultaneously (optionally repeatedly). In one embodiment the components are administered sequentially (optionally repeatedly). In one embodiment the components are administered separately (optionally repeatedly).

The skilled person will understand that where separate formulations of the agents in a combination are administered sequentially or serially, that this could be administration of the agents in any order.

Where the administration of separate formulations is sequential or separate, the delay in administering the second (or subsequent) formulation should not be such as to lose the beneficial therapeutic effect of the combination treatment. Ideally, the two agents will be present within the treated subject substantially at the same time. Suitably, the two (or more) agents will be administered to the subject in the same treatment regime.

### Cancers

The various aspects of the invention that use cancer cells, or are directed to methods or uses for treating cancer, apply to any cancer. Suitably, the cancer is selected from the group consisting of: leukaemia, lymphoma, multiple myeloma, lung cancer, liver cancer, breast cancer, head and neck cancer, neuroblastoma, thyroid carcinoma, skin cancer (including melanoma), oral squamous cell carcinoma, urinary bladder cancer, Leydig cell tumour, biliary cancer, such as cholangiocarcinoma or bile duct cancer, brain cancer, pancreatic cancer, colon cancer, colorectal cancer and gynaecological cancers, including ovarian cancer, endometrial cancer, fallopian tube cancer, uterine cancer and cervical cancer, including epithelia cervix carcinoma. In suitable embodiments, the cancer is leukaemia and can be selected from the group consisting of acute lymphoblastic leukaemia, acute myelogenous leukaemia (also known as acute myeloid leukaemia or acute non-lymphocytic leukaemia), acute promyelocytic leukaemia, acute lymphocytic leukaemia, chronic myelogenous leukaemia (also known as chronic myeloid leukaemia, chronic myelocytic leukaemia or chronic granulocytic leukaemia), chronic lymphocytic leukaemia, monoblastic leukaemia and hairy cell leukaemia. In further preferred embodiments, the cancer is acute lymphoblastic leukaemia. In a suitable embodiment the cancer is lymphoma, which may be selected from the group consisting of: Hodgkin's lymphoma; non-Hodgkin lymphoma; Burkitt's lymphoma; and small lymphocytic lymphoma.

In particular embodiments, the methods and uses disclosed herein provide a precision medicine approach, such as one that targets a particular type of tumour, or sub-set of patients with a particular tumour, or particular stage of tumour, or even an individual patient.

Suitably the treatment of such cancers may achieve effective treatment of the cancer by preventing or treating the development of the cancer, by preventing or treating the progression of the cancer, by preventing or treating the recurrence of the cancer, or by preventing or treating the propagation (including metastasis) of the cancer.

Suitably, the treatment induces the host (subject to whom the treatment is administered) to elicit an immune response against the cancer cells retarding their growth or causing them to be killed. The immune response may be humoral or cell mediated.

### Vaccine Design and Synthesis

According to a ninth aspect of the invention there is provided a method for selecting peptides for inclusion in a tumour vaccine, comprising contacting a tumour cell sample with a PRMT5 inhibitor and determining the expression level of one or more lncRNA gene encoded peptides by the contacted (treated) cells and selecting one or more peptides that are dysregulated for inclusion in a tumour vaccine. Suitably, the tumour cell sample is or has been taken from a patient with a cancer/tumour. By dysregulated we mean upregulated or down regulated compared to the expression in the absence of contacting with the PRMT5 inhibitor. Suitably there is at least a 20%, at least a 30%, at least a 40%, at least a 50%, at least a 60%, or at least a 70% deviation from the expression level the absence of contacting with the PRMT5 inhibitor. In a particular embodiment, the expression level (e.g. transcript level) is upregulated, such as by at least 30%.
Optionally, the dysregulated transcripts are tested to see if they encode immunogenic peptides, e.g if the encoded peptides are presented or likely to be presented on cell surfaces via major histocompatibility complexes (MHCs), e.g. MHC class1. Such testing can involve use of an immunopeptidomic analytical approach such as described in the Examples. Alternatively, a prediction of whether the peptide binds MHC class 1 molecles can be performed (e.g. HLA class I peptides prediction using NetMHC4.0 online algorithm)

Suitably, a peptide can be predicted to be immunogenic using one or more in silico algorithms such as NetMHC4.0 online algorithm or EpiQuest-B.

A peptide can be determined to be immunogenic by a variety of ways, including injection into a host animal and detection of antibodies, or by immuno-peptidomic analysis, for example by the immuno-peptidomic mass spectrometry (MS) technique used in the Examples. Immunogenic peptides induced after a tumour cell is contacted with a PRMT5 inhibitor will bind to MNC class I molecules.

Immunoprecipitation with antibodies specific to MHC class I alleles followed by determination of MHC bound peptides by mass spectrometry analysis can be carried out.

In an embodiment, the method involves determining the expression levels of the lncRNA gene encoded peptides from multiple sources of a single tumour type so as to select the appropriate peptides for inclusion in a vaccine that can be used on any patient with that type of cancer.

Peptides which are identified to be differentially expressed when the cells are contacted with a PRMT5 inhibitor can be tested to determine if they are presented or likely to be presented on cell surfaces via MHC, (such as MHC class I). By putatively presented we mean those that are predicted to be presented using a suitable algorithm (such as NetMHC4.0). Peptides which are predicted to be or are demonstrated to be immunogenic can be selected for inclusion in a vaccine.

Thus, in a variation of the ninth aspect of the invention there is provided a method for selecting peptides for inclusion in a tumour vaccine, comprising contacting cells of a tumour type with a PRMT5 inhibitor and determining the expression level of one or more lncRNA gene encoded peptides by the contacted cells and selecting one or more peptides that are dysregulated, preferably upregulated inclusion in a tumour vaccine. The peptides may be any of those of the first aspect of the invention. Optionally, the peptides determined to be dysregulated are assessed to determine if they are presented or likely to be presented on cell surfaces via MHC, and thus immunogenic. Those that are presented or likely to be presented on cell surfaces via MHC, such as MHC class I are selected for inclusion in a tumour vaccine.

In another variation of the ninth aspect of the invention there is provided method for selecting one or more peptides for inclusion in a tumour vaccine, comprising:
(i) contacting a tumour cell sample with a PRMT5 inhibitor and identifying those lncRNA transcripts that are differentially up- and down-regulated following contact with the PRMT5 inhibitor;
(ii) determining whether the differentially regulated lncRNA gene transcripts identified in step (i) encode immunogenic peptides;
(iii) selecting one or more peptides determined as being immunogenic in step (ii) for inclusion in a tumour vaccine.

In another variation of the ninth aspect of the invention there is provided method for selecting one or more peptides for inclusion in a tumour vaccine, comprising:
(i) contacting a tumour cell sample with a PRMT5 inhibitor;
(ii) preparing a tumour cell lysate from the PRMT5 contacted tumour cell sample of (i);
(iii) contacting the tumour cell lysate with an MHC class 1 specific antibody or antibody fragment thereof bound to a resin on a column; and
(iv) selecting one or more peptides bound peptides for inclusion in a tumour vaccine.

Suitably, the bound peptides in step (ii) are elued from the column and then analysed and identified by mass spectrometry. Following this method, a vaccine comprising the selected peptides is generated.

Thus, according to a tenth aspect of the invention there is provided a method for making a vaccine, comprising selecting the peptides for inclusion into the vaccine according to the ninth aspect of the invention and generating a vaccine capable of presenting said peptides.

The skilled person will appreciate that there are many suitable examples of tumour cell samples that can be employed. Suitably such a sample may include cells from the cancer or pre-cancerous condition. A suitable biological sample may be a tissue sample, such as a sample from a biopsy or surgical resection, or a biofluid sample that comprises tumour cells, such as blood, plasma, serum, sputum, saliva, pleural effusion, ascites, urine and the like. The sample may be fresh, frozen or paraffin embedded.

### PRMT5 Inhibitor Treatments

Protein arginine methyltransferase (PRMT) 5 (PRMT5) is responsible for the mono-methylation and symmetric dimethylation of arginine, and its expression level and methyl transferring activity have been demonstrated to have a close relationship with tumorigenesis, development and poor clinical outcomes of human cancers. PRMT5 is over-expressed in a wide variety of cancers and has been implicated with a key oncogenic role. E2F1 is a significant target for PRMT5, where the methylation mark widens the gene network under E2F1 control.

The inventors have found that pharmacological inhibition of PRMT5 altered the expression of lncRNA genes and consequently antigen presentation by tumour cells. The delayed tumour growth apparent upon PRMT5 inhibition reflected an influx of lncRNA-derived peptide-specific cytotoxic CD8 T cells into the tumour micro-environment and helper CD4 T lymphocytes.

According to an eleventh aspect of the invention there is provided a PRMT5 inhibitor for use in the treatment of cancer by stimulating an immune response.

Thus, administration of a PRMT5 inhibitor induced a host immune response against tumour cells. This represents a new mechanism of action and new clinical approach for treating cancer.

The immune response could be a humoral response and/or cell-mediated response, e.g. adaptive cell mediated response. Suitably, when the PRMT5 inhibitor is administered to a subject it stimulates the subject to production of CD8+ T-cells and helper CD4 T lymphocytes.

Any therapeutic agent capable of inhibiting PRMT5 can be employed in this tenth aspect of the invention. In particular embodiments, the PRMT5 inhibitor can be selected from the group consisting of: an antibody, an siRNA, an antisense oligonucleotide (ASO) or a small molecule compound.

Suitably, the PRMT5 inhibitor is a small molecule compound selected from the group consisting of: GSK3326595 (pemrametostat), PF-6939999, JVNJ-64619178 (onametostat), LLY-283 and PRT543.

PRMT5 inhibitors compounds with distinct chemophores are known. DeFreitas et al (2019) reviews some of the PRMT5 inhibitors, recites their structures and outlines their mechanism of action.

The following table lists some of the patent publication filed by various pharmaceutical companies, and others directed to PRMT5 inhibitors which could be used in the invention.

| | |
|---|---|
| Epizyme Inc & | WO 2016/022605 |
| GSK | |
| Epizyme Inc | WO 2015/200680, WO 2015/200677, WO 2014/100730, WO |
| | 2014/100734, WO 2014/100719, WO 2014/100764, WO |
| | 2014/100695, WO 2014/100716, WO 2014/062720 |
| Eli Lilly | WO 2016/178870 |
| Merck Sharp & | WO 2021/126728, WO 2020/033285, WO 2020/033288, WO |
| Dohme | 2019/094311, WO 2021/126731, WO 2020/033282, WO |
| | 2021/126732, WO 2019/094312, WO 2021/126729, WO |
| | 2020/033284 |
| Prelude | WO 2018/160855, WO 2018/075601, WO 2019/178368, WO |
| Therapeutics | 2018/152548, WO 2019/084470, WO 2018/160824, WO |
| | 2019/032859, WO 2019/085818, WO 2018/085833, WO |
| | 2018/152501, WO 2021/055797, WO 2020168125 |
| Janssen | WO 2017/153186, WO 2018/065365, WO 2017/032840, WO |
| Pharmaceutica | 2019/110734 |
| Lupin Limited | WO 2021/111322, WO 2019/116302, WO 2020/250123 |
| Bayer Akt | WO 2019/002074 |
| Angex Pharma | WO 2019/112719, WO 2020/243178 |
| SK Biopharma | WO 2021/080359, WO 2021/066578 |
| Amgen | WO 2021/163344 |
| Pfizer | WO 2021/140427 |
| Others | WO 2021/207052, WO 2021/088992, WO 2020/198323, WO |
| | 2020/259478, WO 2020/205660, WO 2018/161922, WO |
| | 2019/165189, WO 2018/081451, WO 2019/173804, WO |
| | 2017/153513, WO 2016/145150, WO 2019/180628, WO |
| | 2021/050915, WO 2014/145214, WO 2017/153515, WO |
| | 2021/202480, WO 2019/180631, WO 2016/034675, WO |
| | 2017/153518, WO 2019/102494, WO 2016/034673, WO |
| | 2021/068953, WO 2017/218802, WO 2020/205867, WO |
| | 2016/034671, WO 2011/079236 |

Compound 208 in WO 2014/100719 (Epizyme) is GSK3326595 (pemrametostat).

The compound of Example 2 in WO 2016/178870 (Eli Lilly) is LLY-283

Compound 80 in WO 2017/032840 (Janssen Pharmaceuticals) is JNJ-64619178.

See also De Freitas et al (2019).

Other suitable PRMT5 inhibitors include:
(1) WO 2018/167269 (Argonaut Therapeutics Limited) which disclose compounds of formula I, or a salt, solvate or hydrate thereof,

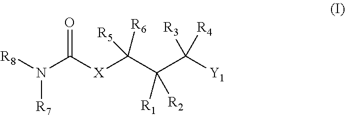

wherein,
R₁, R₃, R₄, R₅ and R₆ are each independently selected from hydrogen and C₁₋₃ alkyl;
R₂ is selected from hydrogen and R₁₄;
X is O or NR₉, where R₉ is hydrogen or a C₁₋₃ alkyl;
Y₁ is a group selected from one of formula A and B,

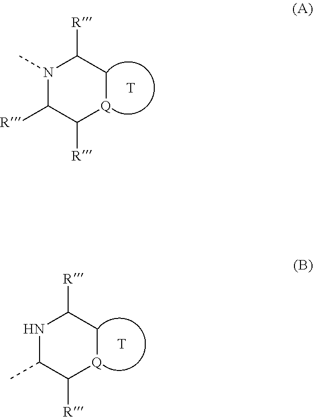

where each R′″ is independently selected from H and C₁₋₃ alkyl;
Q is C or N;
T is selected from a fused phenyl group and a fused 5- or 6-membered heteroaryl group, wherein each group is optionally substituted with one or more substituents selected from halo and C₁₋₃ alkyl; and
R₇ and R₈ are taken together with the intervening nitrogen atom to form a 3-12 membered heterocycloalkyl ring, wherein the 3-12 membered heterocycloalkyl ring is optionally substituted with one or more R₁₀; and/or optionally fused to one or more C₆₋₁₂ aryl, C₅₋₁₂ heteroaryl, C₃₋₈ cycloalkyl and 3-12 membered heterocycloalkyl rings, wherein each fused C₆₋₁₂ aryl, C₅₋₁₂ heteroaryl, C₃₋₈ cycloalkyl and 3-12 membered heterocycloalkyl ring is optionally substituted with one or more R₁₄;
R₁₀ is selected from a group of the formula L₁-L₂-R₁₁ or L₂-L₁-R₁₁, where L₁ is a linker of the formula—[CR₁₂R₁₃]ₙ—, where n is an integer of from 0 to 3 and R₁₂ and R₁₃ are in each instance each independently selected from H and C₁ to C₂ alkyl,
where L₂ is absent or a linker that is selected from O, S, SO, SO₂, N(R′), C(O), C(O)O, [O(CH₂)ᵣ]ₛ, [(CH₂)ᵣO]ₛ, OC(O), CH(OR′), C(O)N(R′), N(R′)C(O), N(R′)C(O)N(R′), SO₂N(R′) or N(R′)SO₂, where R′ and R″ are each independently selected from hydrogen and a C₁ to C₂ alkyl, and where r is 1 or 2 and s is 1 to 4,
R₁₁ is independently selected from hydrogen, CN, NO₂, hydroxyl, ═O, halogen, C₁₋₆ haloalkyl, C₁₋₆ haloalkoxy, C₁₋₆ alkyl, O—C₁₋₆ alkyl, C₃₋₆ cycloalkyl, C₆₋₁₂ aryl, C₅₋₁₂ heteroaryl, 3-10 membered heterocycloalkyl, —C(═O)R^{d}, —C(═O)OR^{d}, —C(═O)NR^{e}R^{d}, —C(O)C(═O)R^{d}, —NR^{e}R^{d}, —NR^{e}C(═O)R^{d}, —NR^{e}C(═O)OR^{d}, —NR^{e}C(═O)NR^{e}R^{d}, —NR^{e}S(═O)₂R^{d}, —NR^{e}S(═O)₂NR^{e}R^{d}, —OR^{d}, —SR^{d}, —OC(═O)R^{d}, —OC(═O)NR^{e}R^{d}, —OC(═O)OR^{d}, —S(═O)₂R^{d}, —S(═O)R^{d}, —OS(═O)R^{d}, —OS(═O)₂R^{d}, —OS(═O)₂OR^{d}, —S(═O)NR^{e}R^{d}, —OS(═O)₂NR^{e}R^{d}, and —S(═O)₂NR^{e}R^{d}, wherein, where R₁₁ is independently selected from C₃₋₆ cycloalkyl, C₆₋₁₂ aryl, C₅₋₁₂ heteroaryl and 3-10 membered heterocycloalkyl, each C₃₋₆ cycloalkyl, C₆₋₁₂ aryl, C₅₋₁₂ heteroaryl and 3-10 membered heterocycloalkyl is optionally substituted with one or more R₁₄;
each R^{a} and R^{b} is independently selected from hydrogen and C₁₋₆ alkyl;
each R^{d} is independently selected from hydrogen, hydroxyl, halogen, CN, C₁₋₆ haloalkyl, 3-7 membered heterocycloalkyl, C₃₋₆ cycloalkyl, C₁₋₆ alkyl, O—C₁₋₆ alkyl and C₆₋₁₁ aryl, wherein said C₁₋₆ alkyl, C₆₋₁₁ aryl, 3-7 membered heterocycloalkyl and C₃₋₆ cycloalkyl are optionally substituted with one or more groups selected from hydroxyl, ═O, halogen, CN, COR^{a}, NR^{a}R^{b}, C₁₋₆ haloalkyl, C₃₋₆ cycloalkyl, C₆₋₁₁ aryl, 3-7 membered heterocycloalkyl, C₁₋₆ alkyl and O—C₁₋₆ alkyl;
each R^{e} is independently selected from hydrogen, hydroxyl, halogen, CN, C₁₋₆ haloalkyl, C₃₋₆ cycloalkyl, C₁₋₆ alkyl and O—C₁₋₆ alkyl; or
R^{e} and R^{d}, when attached to the same atom, together with the atom to which they are attached form a 3-7 membered heterocycloalkyl ring, optionally substituted with one or more substituent selected from hydroxyl, ═O, halogen, CN, COR^{a}, NR^{a}R^{b}, C₁₋₆ haloalkyl, C₃₋₆ cycloalkyl, C₆₋₁₁ aryl, 3-7 membered heterocycloalkyl, C₁₋₆ alkyl and O—C₁₋₆ alkyl; and
R¹⁴ is independently selected from halo, CN, NO₂, hydroxyl, ═O, halogen, C₁₋₆ haloalkyl, C₁₋₆ haloalkoxy, C₁₋₆ alkyl, O—C₁₋₆ alkyl, C₃₋₆ cycloalkyl, C₆₋₁₂ aryl, 5-6 membered heteroaryl, 3-7 membered heterocycloalkyl, C₁₋₆alkylC₆₋₁₂aryl, —C(═O)R^{d}, —C(═O)OR^{d}, —C(═O)NR^{e}R^{d}, —C(O)C(═O)R^{d}, —NR^{e}R^{d}, —NR^{e}C(═O)R^{d}, —NR^{e}C(═O)OR^{d}, —NR^{e}C(═O)NR^{e}R^{d}, —NR^{e}S(═O)₂R^{d}, —NR^{e}S(═O)₂NR^{e}R^{d}, —OR^{d}, —SR^{d}, —OC(═O)R^{d}, —OC(═O)NR^{e}R^{d}, —OC(═O)OR^{d}, —S(═O)₂R^{d}, —S(═O)R^{d}, —OS(═O)R^{d}, —OS(═O)₂R^{d}, —OS(═O)₂OR^{d}, —S(═O)NR^{e}R^{d}, —OS(═O)₂NR^{e}R^{d}, and —S(═O)₂NR^{e}R^{d}.
(2) WO 2018/167276 (Argonaut Therapeutics Limited) which disclose compounds of formula I, or a salt, solvate or hydrate thereof,

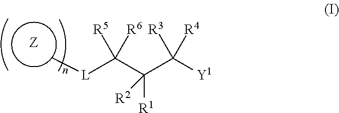

wherein,
Y¹ is a group selected from one of formula A and B,

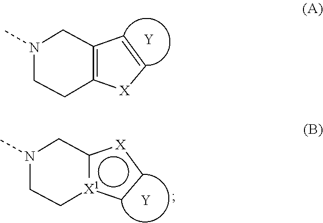

X is selected from O, S, CH and NR⁷;
X¹ is selected from C and N;
Y is selected from a fused aryl group and a fused heteroaryl group, where each group is optionally substituted with one or more R¹;
n is 1 and L is selected from —(CH₂)ₚN(R^{a})C(O)—, —(CH₂)ₚC(O)N(R^{a})—, —(CH₂)ₚN(R^{a})S(O_{q})—, —(CH₂)ₚS(O_{q})N(R^{a})—, —(CH₂)ₚN(R^{b})C(O)N(R^{b})—, —(CH₂)ₚN(R^{c})C(O)O—, and —(CH₂)ₚOC(O)N(R^{c})—; or
n is 0 and L is selected from R^{d}(R^{e})NC(O)—, —R^{d}(R^{e})NC(O)N(R^{b})—, R^{d}(R^{e})NC(O)O—, R^{d}(R^{e})NS(O_{q}) and R^{d}(R^{e})N—;
p is a number selected from 0, 1, 2 and 3;
q is a number selected from 1 and 2;
Z is selected from C₆₋₁₀ aryl optionally substituted by one or more R¹⁰, (C₇₋₁₆)alkylaryl optionally substituted by one or more R¹⁰, C₃₋₁₁cycloalkyl optionally substituted by one or more R¹⁰, (C₄₋₁₇)cycloalkylalkyl optionally substituted by one or more R¹⁰, 3-15 membered heterocycloalkyl optionally substituted by one or more R¹⁰, 4-21 membered alkylheterocycloalkyl optionally substituted by one or more R¹⁰ 5-15 membered heteroaryl optionally substituted by one or more R¹⁰, and 6-21 membered alkylheteroaryl optionally substituted by one or more R¹⁰;
R¹ is selected from hydrogen, halogen, —NR^{e}R^{d}, OR^{f}, and C₁₋₆ alkyl optionally substituted with one or more R⁹;
R² is selected from hydrogen, halogen and C₁₋₆ alkyl optionally substituted with one or more R⁹;
R³, R⁴, R⁵ and R⁶ are independently selected from hydrogen, halogen and C₁₋₆ alkyl optionally substituted with one or more R⁹;
R⁷ is selected from hydrogen, hydroxyl, C₁₋₆ alkyl, C₁₋₆ haloalkyl, phenyl and C₃-6 cycloalkyl, wherein said C₁₋₆ alkyl, phenyl and C₃₋₆ cycloalkyl are optionally substituted by one or more substituents selected from hydroxyl, halogen, ═O, CN, COR^{a}, NR^{a}R^{b}, C₁₋₆ haloalkyl, C₃₋₆ cycloalkyl, C₆₋₁₁ aryl, 3-7 membered heterocycloalkyl, C₁₋₆ alkyl and O—C₁₋₆ alkyl;
each R⁹ is independently selected from hydrogen, hydroxyl, halogen, CN, C₁₋₆ haloalkyl, 3-7 membered heterocycloalkyl, C₃₋₆ cycloalkyl, C₁₋₆ alkyl, O—C₁₋₆alkyl and phenyl, wherein said C₁₋₆alkyl, phenyl, 3-7 membered heterocycloalkyl and C₃₋₆ cycloalkyl are optionally substituted with one or more groups selected from hydroxyl, ═O, halogen, CN, NR^{a}R^{b}, COR^{a}, C₁₋₆ haloalkyl, C₃₋₆ cycloalkyl, phenyl, 3-7 membered heterocycloalkyl, C₁₋₆ alkyl and O—C₁₋₆ alkyl;
each R¹⁰ is independently selected from hydrogen, hydroxyl, ═O, halogen, CN, C₁₋₆ haloalkyl, C₁₋₆ haloalkoxy, C₁₋₆ alkyl, O—C₁₋₆ alkyl, C₃₋₆ cycloalkyl, phenyl, 5-6 membered heteroaryl, 3-7 membered heterocycloalkyl, —C(═O)R^{d}, —C(═O)OR^{d}, —C(═O)NR^{e}R^{d}, —C(O)C(═O)R^{d}, —NR^{e}R^{d}, —NR^{e}C(═O)R^{d}, —NR^{e}C(═O)OR^{d}, —NR^{e}C(═O)NR^{e}R^{d}, —NR^{e}S(═O)₂R^{d}, —NR^{e}S(═O)₂NR^{e}R^{d}, —OR^{d}, —SR^{d}, —OC(═O)R^{d}, —OC(═O)NR^{e}R^{d}, —OC(═O)OR^{d}, —S(═O)₂R^{d}, —S(═O)R^{d}, —OS(═O)R^{d}, —OS(═O)₂R^{d}, —OS(═O)₂OR^{d}, —S(═O)NR^{e}R^{d}, —OS(═O)₂NR^{e}R^{d}, and —S(═O)₂NR^{e}R^{d}, where said C₃₋₆ cycloalkyl, C₁₋₆alkyl, phenyl, 5-6 membered heteroaryl and 3-7 membered heterocycloalkyl are optionally substituted with one or more groups selected from hydroxyl, halogen, ═O, CN, C₁₋₆ haloalkyl, C₁₋₆ haloalkoxy, C₃₋₆ cycloalkyl, C₁₋₆ alkyl and O—C₁₋₆alkyl;
R¹¹ is selected from hydrogen, hydroxyl, halogen, CN, NR^{a}R^{b}, C₁₋₆ haloalkyl, 3-7 membered heterocycloalkyl, C₃₋₆ cycloalkyl, C₁₋₆ alkyl, O—C₁₋₆alkyl and phenyl, wherein said C₁₋₆alkyl, phenyl, 3-7 membered heterocycloalkyl and C₃₋₆ cycloalkyl are optionally substituted with one or more groups selected from hydroxyl, ═O, halogen, CN, COR^{a}, NR^{a}R^{b}, C₁₋₆ haloalkyl, C₃₋₆ cycloalkyl, C₆₋₁₁ aryl, 3-7 membered heterocycloalkyl, C₁₋₆ alkyl and O—C₁₋₆alkyl;
each R^{a}, R^{b} and R^{c} is independently selected from hydrogen and C₁₋₆alkyl;
each R^{d} is independently selected from hydrogen, hydroxyl, halogen, CN, C₁₋₆ haloalkyl, 3-7 membered heterocycloalkyl, C₃₋₆ cycloalkyl, C₁₋₆ alkyl, O—C₁₋₆alkyl and C₆₋₁₁ aryl, wherein said C₁₋₆ alkyl, C₆₋₁₁ aryl, 3-7 membered heterocycloalkyl and C₃₋₆ cycloalkyl are optionally substituted with one or more groups selected from hydroxyl, ═O, halogen, CN, COR^{a}, NR^{a}R^{b}, C₁₋₆ haloalkyl, C₃₋₆ cycloalkyl, C₆₋₁₁ aryl, 3-7 membered heterocycloalkyl, C₁₋₆ alkyl and O—C₁₋₆ alkyl;
each R^{e} is independently selected from hydrogen, hydroxyl, halogen, CN, C₁₋₆ haloalkyl, C₃₋₆ cycloalkyl, C₁₋₆ alkyl and O—C₁₋₆ alkyl; or
R^{e} and R^{d}, when attached to the same atom, together with the atom to which they are attached form a 3-7 membered heterocycloalkyl ring, optionally substituted with one or more substituent selected from hydroxyl, ═O, halogen, CN, COR^{a}, NR^{a}R^{b}, C₁₋₆ haloalkyl, C₃₋₆ cycloalkyl, C₆₋₁₁ aryl, 3-7 membered heterocycloalkyl, C₁₋₆ alkyl and O—C₁₋₆ alkyl; and
R^{f} is independently selected from hydrogen and C₁₋₆ alkyl optionally substituted with one or more substituents selected from hydroxyl, halogen, CN, COR^{a}, NR^{a}R^{b}, C₁₋₆ haloalkyl, C₃₋₆ cycloalkyl, phenyl, 3-7 membered heterocycloalkyl and O—C₁₋₆ alkyl.
(3) GB2108383.7 (Argonaut Therapeutics Limited) which discloses compounds of formula (1) or a deuterated form, salt, solvate, or hydrate thereof,

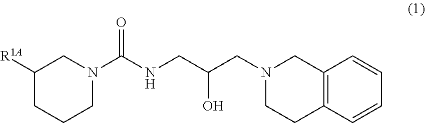

wherein:
R^{1A} is represented by formula (A1),

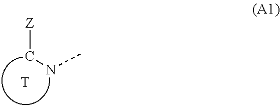

Z is ═O;
T taken together with the intervening carbon and nitrogen atoms (e.g. shown in formula (A1)) is selected from a monocyclic 5- to 7-membered heterocycloalkyl group, a fused bicyclic 6- to 10-membered heterocycloalkyl group and a bridged bicyclic 6- to 9-membered heterocycloalkyl group, wherein each of the monocyclic 5- to 7-membered heterocycloalkyl group, the fused bicyclic 6- to 10-membered heterocycloalkyl group and the bridged bicyclic 6- to 9-membered heterocycloalkyl group is optionally substituted with one or more R^{S1};
R^{S1} is selected from C₁₋₆alkyl, C₂₋₆alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}; and
R^{S2} is selected from hydroxy, halo, CN and nitro.

Any of these PRMT5 compounds can be used in the present invention.

In particular embodiments, the PRMT5 inhibitor for use in the present invention is a small molecule compound selected from the group consisting of: GSK3326595 (pemrametostat), PF-6939999, JVNJ-64619178 (onametostat), LLY-283 and PRT543.

Effective anti-cancer treatments using, for example, a chemotherapeutic agent or radiotherapy, are hampered by the ability of cancer cells to develop resistance to these treatments. The finding that PRMT5 inhibitor can also stimulate a host immune response against a tumour cell would permit the use of a PRMT5 inhibitor to treat a patient or patient group whose cancers have developed resistance to the direct cell targeting effect of the PRMT5.

The following Examples and associated Figures serve to illustrate the invention. These Examples and Figures are in no way intended to limit the scope of the invention, but rather as examples from which equivalents will be recognized by those of ordinary skill in the art.

Unless it is apparent from the context, each of the embodiments listed above can be applied for use in any of the aspects of the invention.

### EXAMPLES

Protein arginine methyltransferase (PRMT) 5 is over-expressed in a wide variety of cancers and has been implicated with a key oncogenic role. E2F1 is a significant target for PRMT5, where the methylation mark widens the gene network under E2F1 control. We show here that the PRMT5-E2F1 axis has an additional unexpected role in controlling expression from the non-coding genome where a large group of long non-coding (Inc) genes are direct transcription targets. Analysis of antigen presentation by tumour cells identified that many MHC class I protein-associated peptides were derived from small open reading frames in lncRNA genes. Further, pharmacological inhibition of PRMT5 and manipulation of E2F1 activity altered the expression of lncRNA genes and consequently antigen presentation by tumour cells. The delayed tumour growth apparent upon PRMT5 inhibition reflected an influx of lncRNA-derived peptide-specific cytotoxic CD8 T cells into the tumour micro-environment. When presented to the immune system as a stand-alone therapeutic vaccine, lncRNA-derived peptides were found to be immunogenic and, significantly, drive a potent anti-tumour immune response. These results show that PRMT5 links the E2F pathway to the non-coding genome and the antigenic landscape of tumour cells. Pharmacological control of PRMT5 activity thus offers a therapeutic strategy to influence the immunogenicity of tumour cells.

### Materials and Methods

### Cell Line Generation, Culture, and Compound Treatments

Human p53^{-/-} HCT116 E2F1 CRISPR and CAS9 control cells have been described previously (Barczak et al., 2020). Mouse CT26 cells were acquired from ATCC (CRL-2638). Cells were cultured in Dulbecco's modified Eagle medium (DMEM) (Sigma-Aldrich, St. Louis, MO, US) supplemented with 10% fetal bovine serum (Labtech, Heathfield, UK) and 1% penicillin/streptomycin (Gibco, Life Technologies, Carlsbad, CA, USA). All cell lines were tested for mycoplasma contamination before use. Selective PRMT5 inhibitor (T1-44) (synthesised by Argonaut Therapeutics Ltd, Oxford UK) has been described and characterised previously (Barczak et al., 2020) and was used for 48 hours at 1 μM final concentration unless otherwise stated.

### Plasmid/siRNA Transfections

Plasmid transfections were performed for 48 hours using the GeneJuice transfection reagent (Novagen), as per the manufacturer's instructions. RNA interference was performed with 25 nM siRNA for 72 hours using the Oligofectamine transfection reagent (Invitrogen), as per the manufacturer's instructions. Sequences for siRNA are as follows: nontargeting control, 5′-AGCUGACCCUGAAGUUCUU-3′ (SEQ ID NO: 195); E2F1 (human), 5′-CUCCUCGCAGAUCGUCAUCUU-3′; E2F1 (mouse)(cat no: EMU075181, Merck) (SEQ ID NO: 196).

### Immunoblots and Antibodies

For immunoblots, cells were harvested in modified RIPA buffer (50 mM tris-HCl pH 7.5, 150 mM NaCl, 1% Igepal CA-630 [v/v], 1 mM EDTA, 1 mM NaF, 1 mM Na₃VO₄, 1 mM AEBSF, protease inhibitor cocktail) and incubated on ice for 30 min prior to SDS-PAGE and transfer to nitrocellulose. The following antibodies were used in immunoblots: β-actin (AC-74, Sigma-Aldrich), E2F1 (Cell Signalling, 3742S), symmetric di-methyl arginine (SDMe) (Cell Signalling, 13222S), FLAG M2 (Sigma, F1804), GFP (D5.1; Cell Signalling, 2956S), GAPDH (Bethyl Laboratories).

### RNA Isolation and Quantitative RT-PCR

RNA was isolated from cells using TRIzol (Thermo Fisher Scientific) or the Direct-zol RNA MiniPrep kit (Zymo Research) according to the manufacturer's instructions. One microgram of total RNA was used for complementary DNA (cDNA) synthesis. Reverse transcription with oligo(dT)20 primer (Invitrogen) was performed using SuperScript III Reverse Transcriptase (Invitrogen) as per the manufacturer's instructions. Quantitative PCR (qPCR) was then carried out in triplicate using the indicated primer pairs and the Brilliant III SYBR Green qPCR Master Mix (Stratagene) on an AriaMx (Agilent) qPCR instrument. Results were expressed as average (mean) fold change compared to control treatments using the ΔΔCt method from three biological repeat samples. Glyceraldehyde-phosphate dehydrogenase (GAPDH) primer sets were used as an internal calibrator. Error bars represent SD unless otherwise indicated.

### RNA Sequencing

WT E2F1, E2F1 Cr HCT116, and CT26 cells were treated with 1 μM concentration of PRMT5 inhibitor (T1-44) or DMSO as a negative control, for 48 h (HCT116) or 72 h (CT26). Total RNA from WT E2F1, WT E2F1 T1-44, E2F1 Cr, E2F1 CrT1-44, CT26, and CT26 T1-44 (triplicates) was isolated using Direct-zol RNA MiniPrep kit (Zymo Research) according to the manufacturer's instructions. Alternatively, RNA isolated from mouse tumours in situ was used for RNA-seq analysis. RNA-sequencing was performed by BGI Genomics. Briefly, an Agilent 2100 Bioanalyzer (Agilent RNA 6000 Nano Kit) was used for RNA sample quality control purposes (RNA concentration, RIN value, 28S/18S, and the fragment length distribution). mRNAs were isolated from total RNA using the oligo(dT) method. Then the mRNAs were fragmented, and first strand/second strand cDNA were synthesized. cDNA fragments were purified and resolved with EB buffer for end reparation and single nucleotide A (adenine) addition. Subsequently, the cDNA fragments were linked with adapters. Those cDNA fragments with suitable size were selected for the PCR amplification. Agilent 2100 Bioanalyzer and ABI StepOnePlus Real-Time PCR System were used in quantification and qualification of those libraries. The RNA sequencing was carried out using Illumina HiSeq Platform, and 5.12 Gb per sample was generated.

### Long Non-Coding RNA (LncRNA) Expression Data Processing

FASTQ files for p53−/− WT E2F1, p53−/− E2F1 Cr HCT116, and CT26 cells treated with PRMT5 inhibitor or DMSO control were generated from three biological repeat experiments. These were trimmed to remove adapters and low-quality bases with TrimGalore v.0.4.3 (http://www.bioinformatics.babraham.ac.uk/projects/trim_galore/). LncRNA expression analysis was performed using kallisto (v. 0.44.0) with k-mer length 31 and 100 bootstrap samples. GENCODE mouse lncRNA annotation version M22 and human lncRNA annotation version 34 were used as reference dataset to construct kallisto indices. Differential expression of lncRNA was computed with sleuth package (v 0.30.0). The log 2(fold-change) in expression was computed from estimated counts values (provided by kallisto) averaged across all replicates for a given condition.

Significantly differentially expressed transcripts were identified using FDR threshold (q-value) of 0.05.

HCT116 p53−/− and HCT116 p53−/− E2F1 Cr RNA-seq datasets have been deposited to the Gene Expression Omnibus (GEO) under accession code GSE142430. CT26 and colon26 tumour sample RNA-seq datasets have been deposited to GEO under accession code GSE181401.

Data Processing for Proteomic Mass-Spectrometry (lncRNA-Derived Peptide Databases)

Nucleotide sequences of all lncRNAs expressed at detectable levels in our HCT116 and CT26 RNA-seq datasets (mouse: GENCODE annotation; human: FANTOM 5 and GENCODE annotation) were converted into peptide sequences using 3-frame translation. The peptide sequence data were broken down into 3 groups, in accordance with expression values of corresponding lncRNAs: non-expressed (TPM=0); weakly expressed (0.5<TPM<1.0); expressed (TPM>1.0). The non-expressed group were used as a decoy database for MS proteomic experiments (detailed below).

### HLA Class I Immunoprecipitation

Antibodies were sourced from hybridoma supernatants (ATCC® HB-95 and -79, respectively) using a standard purification procedure using Sepharose-protein A beads (Expedeon). 0.5 ml/sample Sepharose-protein A beads (Expedeon) were incubated with 5 mg/sample of W632 antibody (specific for HLA-class I for HCT116), or antibody clone 34.1.2 s (recognising H-2 Kd, Dd, Ld for CT26), for 30 minutes at room temperature. The resin was washed with 10 cv (column bed volumes) of borate buffer (50 mM borate, 50 mM KCl, pH 8.0) and antibodies were cross-linked by adding 10 cv of 40 mM dimethyl pimelimidate in borate buffer (pH8.3) for 30 minutes at room temperature. The reaction was stopped with 10 cv of ice-cold 0.2 M Tris, pH 8.0, followed by a washing step of 10 cv of 0.1 M citrate, pH 3.0, to remove any unbound antibody, and finally equilibrated with 10 cv of 50 mM Tris, pH 8.0.

Cell pellets were lysed in 3 ml lysis buffer (1% IGEPAL 630; 100 mM Tris, pH8.0; 300 mM NaCl; supplemented with complete Protease Inhibitor Cocktail, EDTA-free, Roche) by mild agitation. Samples were incubated for 45 minutes on ice. Lysates were then cleared by sequential centrifugation steps at 500 g for 10 minutes then 20,000 g for 1 h at 4° C. Peptide-HLA class I complexes were captured on the immunoresin by overnight incubation at 4° C. under mild agitation. The lysate was then removed by gravity flow, and the column was washed consecutively with 10 ml wash buffer 1 (0.005% IGEPAL, 50 mM Tris pH8.0, 150 mM NaCl, 5 mM EDTA), 10 ml wash buffer 2 (50 mM Tris pH8.0, 150 mM NaCl), 10 ml wash buffer 3 (50 mM Tris pH8.0, 450 mM NaCl) and 10 ml wash buffer 4 (50 mM Tris pH8.0). Peptide-HLA complexes were eluted by addition of 5 cv of 10% acetic acid.

### HLA Peptide Purification Strategies

Samples were loaded onto a Ultimate 3000 HPLC system (ThermoFisher Scientific) and peptides were separated from larger complex components using a monolithic column (4.6×50 mm ProSwift RP-1 S, ThermoFisher Scientific) by applying a 10 minutes gradient from 2 to 35% buffer B (0.1% TFA in acetonitrile) with a flow rate at 1000 μl/min. Each sample was fractionated in 15 fractions and alternate fractions containing the HLA peptides but not ß2-microglobulin, were pooled in two final fractions. Samples were dried, re-suspended in 20 μl of loading buffer (0.1% TFA, 1% ACN) and stored at −80° C. prior to MS analysis.

### LC-Tandem Mass Spectrometry (LC-MS/MS)

For HCT116 cell samples, HLA peptides were analyzed by either an Orbitrap Fusion Lumos Tribrid mass spectrometer (Thermo Scientific) or a Q Exactive HF-X mass spectrometer (Thermo Scientific). CT26 cell samples were measured on a Q Exactive HF-X (Thermo Scientific). Either mass spectrometer instrument was coupled with an Ultimate 3000 RSLCnano System supplemented with a PepMap C18 column, 2 μm particle size, 75 μm×50 cm (Thermo Scientific). Peptides were eluted using a 60 minute linear gradient of 3% to 25% acetonitrile in 5% DMSO, 0.1% formic acid in water at flow rate of 250 nl/min and 40° C., and introduced into the mass spectrometer using a nano EASY-Spray source at 2000 V (Thermo Scientific). The ion transfer tube was set to 305° C. for both instruments.

For samples analysed by the Orbitrap Fusion Lumos, the resolution for full MS was set at 120,000 with ACG target of 400,000 and scan range of 300-1500 m/z. Precursor selection and isolation were performed using TopSpeed in a 2 s cycle time and 1.2 amu quadrupole isolation width. MS2 resolution was set at 30,000 and peptide ions were accumulated at a maximal injection time of 120 ms with an AGC target of 300,000. Precursor ions were fragmented using high-energy collisional dissociation (HCD): Collision energy was set to 28 for peptides with charge state of 2-4, and set to 32 for singly-charged ions. For samples analysed on the Q Exactive HFX, full MS (320-1600 m/z scan range) resolution was set at 120,000, and an AGC target of 300,000. Peptide ions were isolated at 1.6 amu isolation width. MS2 resolution was set to 60,000 at an AGC target of 50,000 and the collision energy was set at an energy of 28 for peptides with a charge state of 2-4 fragmentation of precursor ions and 25 for those with a charge state of 1-4.

### HLA Binding Prediction

HLA class I peptides prediction was performed using NetMHC4.0 online algorithm (http://www.cbs.dtu.dk/services/NetMHC/) and Seq2Logo2.0. (http://www.cbs.dtu.dk/services/NetMHC/) or WebLogo (http://www.weblogo.berkeley.edu/logo.cgi).

Chromatin immunoprecipitation (ChIP)

E2F1 ChIPs were performed as described previously (Carr et al., 2017), using 3 μg of appropriate antibody (control rabbit IgG, anti-E2F1 [A300-766A, Bethyl Laboratories) and pre-blocked protein A beads. The recovered DNA was purified and real-time PCR was performed in triplicate with Brilliant III Ultra-Fast SYBR green QPCR master mix on an AriaMx QPCR instrument (Agilent) using primers flanking proposed E2F sites in gene promoters. DNA occupancy was investigated by calculating the percentage enrichment of input for both the E2F1 ChIP and IgG controls from triplicate biological repeat experiments. In all cases, the presented figure displays SD unless otherwise stated. The CDC6 and actin promoters were used as a positive and negative control for E2F1 occupancy respectively.

Human and Mouse lncRNA Promoter Analysis

LncRNA gene promoter characterisation was performed utilising bioinformatics tools present in UCSC Genome Browser (http://genome.ucsc.edu; GRCh37/h19 assembly) and analysing ChIP-seq data for E2F tracks from the ENCODE project (http://genome.ucsc.edu/ENCODE/) for three cell lines (K562, MCF7, HeLa). The ‘Transcription factor ChIP-seq clusters from ENCODE 3’, ‘Transcription factor ChIP-seq clusters from ENCODE with factorbook motifs’, ‘Transcription factor ChIP-seq peaks from ENCODE 3’, ‘Transcription factor ChIP-seq uniform peaks from ENCODE/Analysis’ and ‘Transcription factor binding sites by ChIP-seq from ENCODE/Stanford/Yale/USC/Harvard’ track tools were used to display E2F1 ChIP-seq peaks or signal as appropriate. Genes were scored as being potential E2F1 targets if ChIP-seq data was apparent within 1000 bp wide regions around the annotated promoter (annotated by GENCODE and FANTOM6). For promoter characterisation in mouse, the GRCm38/mm10 assembly was used, and mouse E2F1 ChIP-seq peak data was loaded as a custom track using data deposited in GEO (GSM288349). ChIP-seq peak coordinates were intersected with 1000 bp wide regions around lncRNA transcription start sites (GENCODE annotation).

### Polysome Profiling

Cells were treated with 100 mg/ml cycloheximide for 10 minutes at 37° C., treated with 1× trypsin-EDTA solution for 10 min and washed twice with ice cold 1×PBS containing 100 mg/ml of cycloheximide. Polysome lysis buffer composed of 20 mM Tris HCl pH 7.4, 5 mM MgCl₂, 100 mM KCl, 100 μg/mL cycloheximide, 1% Triton X-100, 1× RNase inhibitor, and 1× protease inhibitors was used to resuspend cells, followed by 30 min incubation on ice (occasional inverting) and 10 min centrifugation at 12,000 g at 4° C. Sucrose gradients were prepared using 10% and 50% sucrose solutions (sucrose diluted in polysome extraction buffer without Triton X-100 and prepared in RNAse-free conditions) in polypropylene, 13.2-ml tube (Beckman Coulter). The gradient was left at 4° C. overnight to become linear. Clear supernatants from lysed cells were loaded (equal amount—300 μg—of RNA measured by Nanodrop) onto the 10-50% sucrose gradients and centrifuged at 39,000 rpm (190,000 g) (SW40Ti rotor, Beckman Coulter Optima XE) for 90 min at 4° C. Twelve sucrose gradient fractions were separated using manual collection, and the absorbance was measured at 254 nm (NanoDrop (Thermo Fisher Scientific) to record the polysome profile.

### LncRNA ORF Cloning Strategy

For those lncRNAs identified in HCT116 and CT26 cells as giving rise to peptides loaded onto MHC class I, the peptide sequence was initially reverse-translated back to the lncRNA transcript. lncRNA transcripts were then translated in all 3 frames, and potential open reading frames (ORFs) were identified by highlighting all sequences contained between every ATG codon (encoding a start methionine) and a subsequent in frame STOP codon. Any potential ORF that would generate a polypeptide that contained the identified MHC peptide was identified as a sequence for cloning into a plasmid vector expressing a C-terminal FLAG tag (pSF-CMV-NEO-COOH-3×FLAG; OG629, OxGene). Primers were designed to amplify the ORF (minus the STOP codon) and 30 bp upstream sequence (to include any Kozak sequence present in the endogenous transcript) and contained restriction sites for NotI and XhoI/EcoRV as appropriate. A PCR reaction was performed using Phusion High Fidelity DNA Polymerase (M0530S, New England Biolabs) and cDNA from HCT116 or CT26 cells as a template (generated as described above for quantitative RT-PCR). PCR products were purified using a QiAquick PCR purification Kit (Qiagen) and digested with NotI and XhoI/EcoRV (Promega) as appropriate. Digested products were gel purified using a QiAquick Gel Extraction Kit (Qiagen) prior to ligation into digested vector using T4 DNA ligase (New England Biolabs). All plasmids were sequenced to confirm correct cloning prior to use in transfections.

### Functional Genomics Analysis—TCGA

For the analysis of peptide coding lncRNAs transcripts expression levels in human cancers, Xena browser (University of California) was used (https://xena.ucsc.edu/). The TCGA TARGET GTEx dataset was selected, which contained transcript expression data from TCGA (cancer tissue; https://www.cancer.gov/about-nci/organization/ccg/research/structural-genomics/tcga) and Genotype-Tissue Expression (GTEx; healthy tissue; https://gtexportal.org/home/) samples. For subsequent detailed analysis of microsatellite instability and staging, datasets from TCGA were used. Also, Broad Institute Cancer Cell Line Encyclopedia (portals.broadinstitute.org>ccle) was used to analyse the expression of lncRNA genes in colorectal cancer cell lines. Heatmaps were generated utilising Heatmapper tool (http://heatmapper.ca/). For detailed survival analysis, GEPIA2 tool was used (http://gepia2.cancer-pku.cn/#index).

Colon26 Mouse Tumour Model with T1-44 Treatment

All experiments and protocols were approved by the animal welfare body at Charles River Discovery Research Services Germany (where experiment was performed) and the local authorities, and were conducted according to all applicable international, national and local laws and guidelines. Sixteen female Balb/c mice at 6-8 weeks of age (8 mice per group: control and treated) (Charles River Laboratories, Germany) received unilateral subcutaneous injections of 5×10⁵ Colon26 cells in PBS in a total injection volume of 100 μl/mouse. Upon reaching individual tumour volumes of 50-150 mm³, mice were assigned to treatment groups based on tumour volumes aiming at comparable group mean/median tumour volumes. Within 24 hours of randomization, mice were daily treated by oral administration (gavage) with 100 mg/kg (dosing volume 10 ml/kg) of T1-44 using 0.5% Tween/PBS as a vehicle. Body weights and tumour volume [mm³] by caliper measurement were performed twice weekly. Termination of individual mice was conducted at day 19^{th} of the experiment or at >1000 mm³ (unilateral), in case of tumour ulceration or body mass loss at <70% of initial weight. From each group, four snap frozen tumours were collected for RNA isolation and four formalin-fixed samples were prepared for immunohistochemical staining.

### Mice and Vaccination

Groups of 8 week old Balb/c mice (Charles River) were vaccinated i.v with the indicated peptide mix (Table 2), (50 μg/peptide (GenScript)) with 30 μg polyIC and 25 μg anti-CD40 mAb). As a positive control AH peptide (SPSYVYHQF SEQ ID NO: 156) vaccination was used (Huang et al., 1996). Vehicle-only mice were vaccinated with DMSO/PBS plus adjuvants as above. Mice were boosted i.v. 7 days later with the same formulation. Mice were culled 7 days post-boost and their spleens removed (4 mice per group). All animals were housed in specific pathogen-free conditions at the Biomedical Services Building (University of Oxford). All work was performed under UK Home Office license PPL PP3430109 in accordance with the UK Animal (Scientific Procedures) Act 1986. All work was performed by trained and licensed individuals.

| TABLE 2Group | | | | | |
|---|---|---|---|---|---|
| (mouse) | Peptide (SEQ ID NO:) | | | | |
| 1 (1-4, | RGPSHFSRL | KYLRLHERI | SLPVRSLSL | SVPVRVSII | SLPVRSLSL |
| neg) | (SEQ ID | (SEQ ID NO: | (SEQ ID NO: | (SEQ ID NO: | (SEQ ID NO: |
| | NO: 157) | 158) | 159) | 160) | 161) |
| 2 (1-4, | KGPEHKLLL | RGPSLSRDL | SHPQKYERI | TGPRRPQI | RLAQLQTTI |
| neg) | (SEQ ID NO: | (SEQ ID NO: | (SEQ ID NO: | (SEQ ID NO: | (SEQ ID NO: |
| | 162) | 163) | 164) | 165) | 166) |
| 3 (1-4, | RGPLLEKLF | GFLGSNTDI | HPTVPNPYNLL | GSPSLVHQV | SNISYKNGI |
| neg) | (SEQ ID NO: | (SEQ ID NO: | (SEQ ID NO: | (SEQ ID NO: | (SEQ ID NO: |
| | 167) | 168) | 169) | 170) | 171) |
| 4 (1-4, | RPLISIKHGL | LNPSALSAL | RATPEVTLI | YNPILSKL | HIFSLHHF |
| neg) | (SEQ ID NO: | (SEQ ID NO: | (SEQ ID NO: | (SEQ ID NO: | (SEQ ID NO: |
| | 172) | 173) | 174) | 175) | 176) |

### ELispot Assay

The day before culling, Merk Multiscreen 96 well Filter Plates (Merck) were incubated with primary antibody (anti-mouse INFy mAb clone AN18, Mabtech) diluted in sterile PBS (Gibco), at 4° C. The next day, the antibody was removed, plates were washed 4 times with PBS at 250 μl/well, then blocked with 200 μl/well R10 (RPMI (Gibco) supplemented with 10% heat-inactivated FCS, Non-essential amino acids, L-Glutamine and Penicillin/Streptomycin (all from Sigma) for 2 hours at 37° C.). Mice were culled, their spleens removed, and passed through a 40 μm cell strainer (Falcon) and the single cell suspension pelleted by centrifugation. The splenocytes were resuspended in 3 ml ACK lysis buffer (Lonza) for 3-5 mins to lyse the red blood cells, then stopped with 20 ml PBS, followed by centrifugation at 1500 rpm, 5 mins at room temperature. The splenocyte pellet was resuspended in 5 ml R10, counted and the cell concentration adjusted to 4×10⁵/ml. Blocking buffer was removed and replaced with 50 μl of cells which were stimulated with the respective individual peptides (50 μl of peptide at 15 μg/ml) that the group had been vaccinated with. Each peptide was tested in duplicate. Negative control wells contained DMSO only while cells were stimulated with concanavalin A as a positive control. The plates were incubated overnight (15-20 hours) in a 37° C. (5% CO₂) incubator. The cells and peptides were removed and the wells washed 7 times with sterile PBS. Secondary antibody (biotin conjugated mouse anti-INFy, MabTech) diluted 1:2000 in assay diluent (AD) (25 mg/ml BSA in PBS), was added (50 μl/well) and incubated for 2 hours at room temperature. The plates were then washed 4 times with PBS then 50 μl of anti-biotin-Alkaline Phosphotase (Mabtech) diluted 1:750 in AD was added and incubated for 2 hours at room temperature. Plates were washed 4 times with PBS then 50 μl BCP/NBT substrate was added to each well and allowed to develop for 5-10 mins until spots were visible in the positive control wells. Reaction was stopped by rinsing the plates in DI water three times. The rubber bottom was removed and the membrane was rinsed on both sides with DI water then allowed to dry. The spots were quantitated on an ELISPOT counter (AID, Germany).

### Dendritic Cell Vaccine Strategy in Colon26 Mouse Tumour Model

All experiments and protocols were approved by the animal welfare body at WuXi AppTec (HongKong) Limited (where experiment was performed) and the local authorities, and were conducted according to all applicable international, national and local laws and guidelines. Sixteen female Balb/c mice (RRID:IMSR_CRL:547) at 6-8 weeks of age (8 mice per group: control (unpulsed dendritic cells) and peptide pulsed dendritic cells) (Vital River Laboratory Animal Technology Co., LTD) received unilateral subcutaneous injections of 3×10⁵ colon26 cells in PBS in a total injection volume of 100 μl/mouse. Upon reaching individual tumour volumes of 60-80 mm³, mice were assigned to treatment groups based on tumour volumes aiming at comparable group mean/median tumour volumes. Within 24 hours of randomization, mice were vaccinated with 1×10⁶ cells/0.2 mL unpulsed or pulsed dendritic cells intravenously. To prepare the vaccine mouse bone marrow cells were harvested. Next, cells were treated with GM-CSF (250 IU/mL) and IL-4 (5 IU/mL) containing medium, and incubated at 37° C. in 5% CO₂. Medium was half changed at day 3. At day 6, cells were treated with GM-CSF, IL-4 and LPS to mature dendritic cells. After incubation for 24 hours, DC cells were harvested and the phenotype was analysed by FACS (CD11c, CD80, CD86). Then, 2×10⁵ DC cells/mL were pulsed with peptides (tab. X) at 75 μg/ml (15 peptides, 5 μg/mL each) and incubated for 5 h. After harvesting and washing DC cells with medium they were ready for injection. The second vaccination was performed at day 7. Body weights and tumour volume [mm³] were performed by caliper measurement twice weekly. Termination of individual mice was conducted at day 12 of the experiment or at >3000 mm3 (unilateral), in case of tumour ulceration or body mass loss at <80% of initial weight. From each group formalin-fixed samples were prepared for immuno-histochemical staining.

### Immunohistochemical Staining

FFPE slides were washed for 5 min with Histochoice (Sigma Aldrich), followed by two times 3 min washing in 100% Ethanol, 3 min in 70% Ethanol and 5 min in tap water. Next, samples were incubated with antigen retrieval solution (sodium citrate buffer or Tris/EDTA depending on the antibody used) at 99° C. in a water bath for 20 min. After 3× washing with purified water, samples were incubated with freshly made 6% Methanol/H₂0₂ for 15 min, and washed in tap water. In the next steps, slides were washed in 1% PBST for 5 min, blocked in blocking serum solution (Vectastatin ABC kit) for 20 min, washed again in 1% PBST for 5 min and incubated overnight at 4° C. with primary antibody: SDMe (1:5000, Cell signaling), CD8 (1:11000, Abcam), CD4 (1:8000, Abcam), FoxP3 (1:4000, Cell signaling), CD163 (1:5000, Abcam). On the next day, slides were washed with 1% PBST for 5 min followed by 30 min incubation with secondary antibody (Vectastain ABC kit) at room temperature. In the next step ABC solution (VECTASTAIN® ABC-HRP Kit, Peroxidase, Rabbit IgG, PK-4001) was added for 30 min, slides were washed in 1% PBST and incubated with DAB solution (Vector DAB) for 10 min. Then, slides were washed in purified water and counterstained in hematoxylin (Sigma Aldrich). Results were analysed using a Leica microscope and presented as semi-quantitative data using ImageJ software (National Institutes of Health).

### Statistical Analysis

Statistical analyses were performed using two-tailed, unpaired Student's t-test and one way ANOVA test with GraphPad Prism 8 Software (GraphPad Software). Data are shown as means with SE displayed unless otherwise indicated. P values lower than 0.05 were considered significant and are labelled by asterisks (*) for p<0.05, (**) for p<0.01, (***) for p<0.001, and (****) for p s 0.0001.

### Example 1

E2F1 and PRMT5 Control Expression of the Human lncRNA Genes

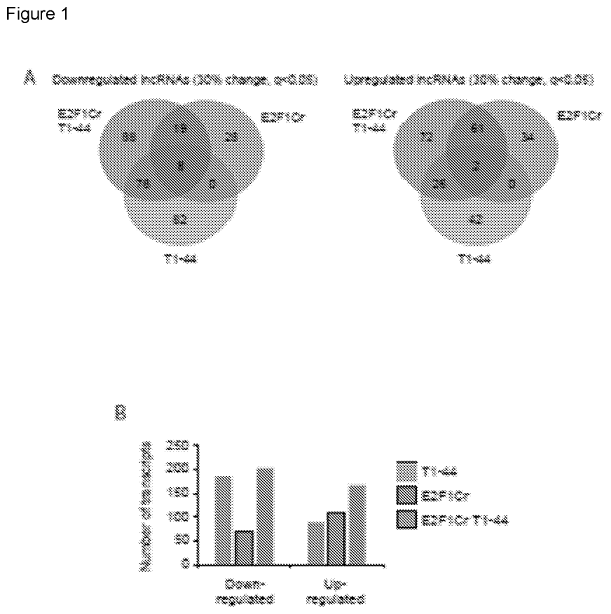

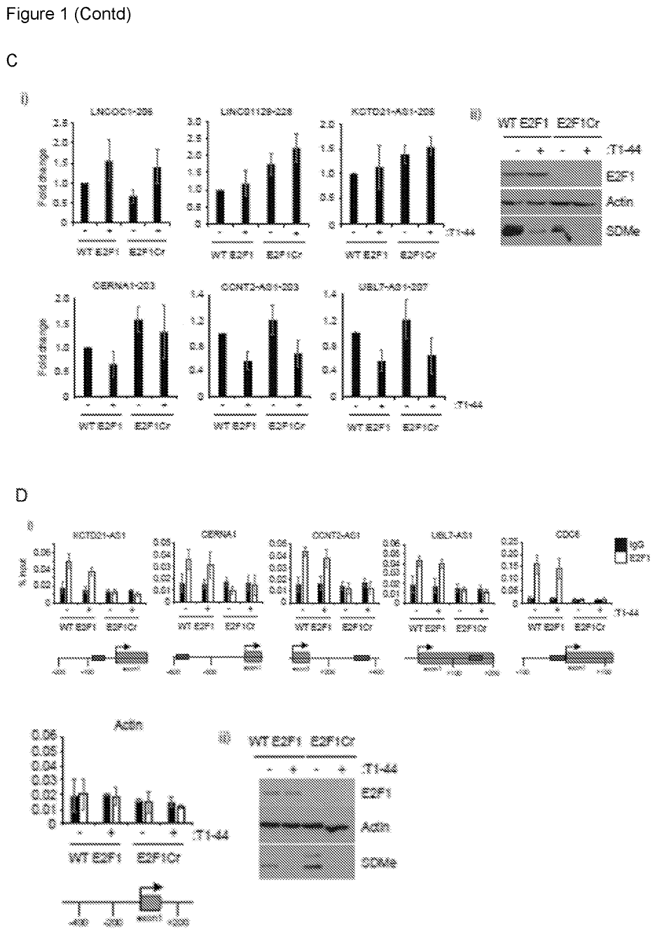

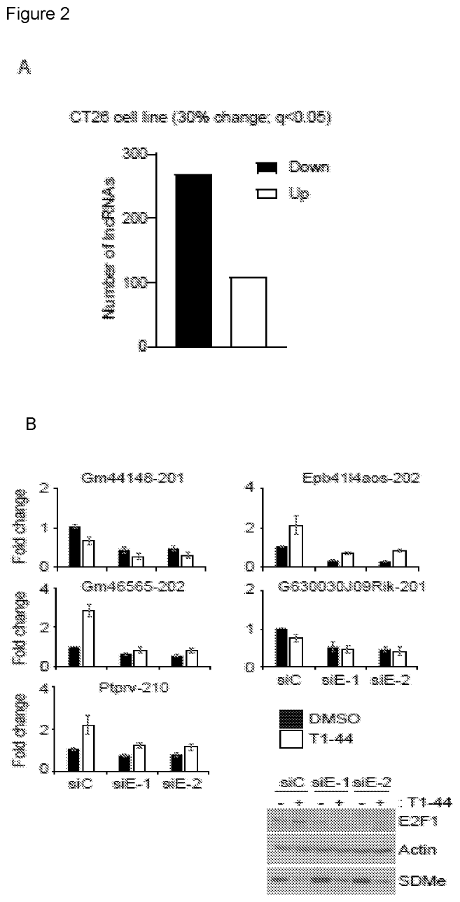

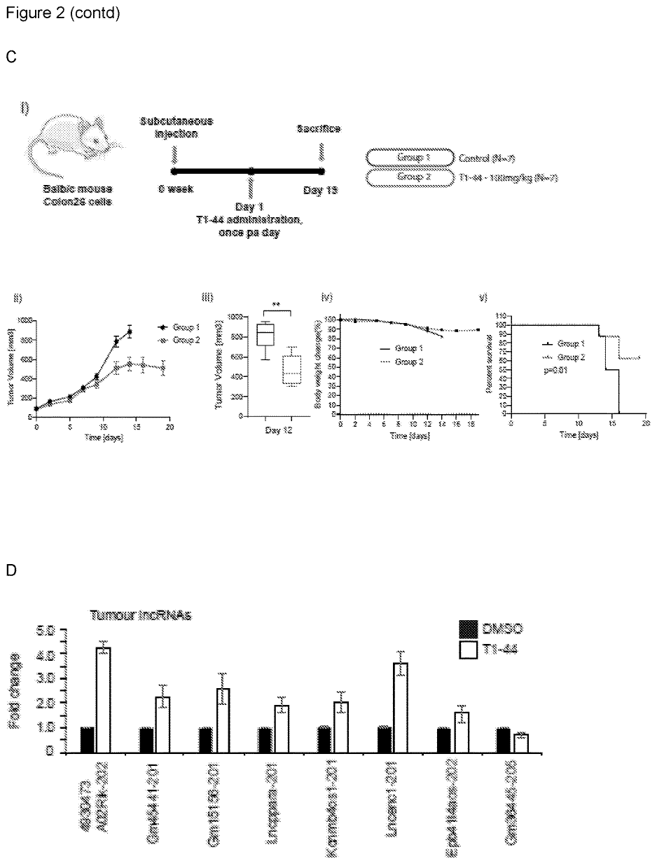

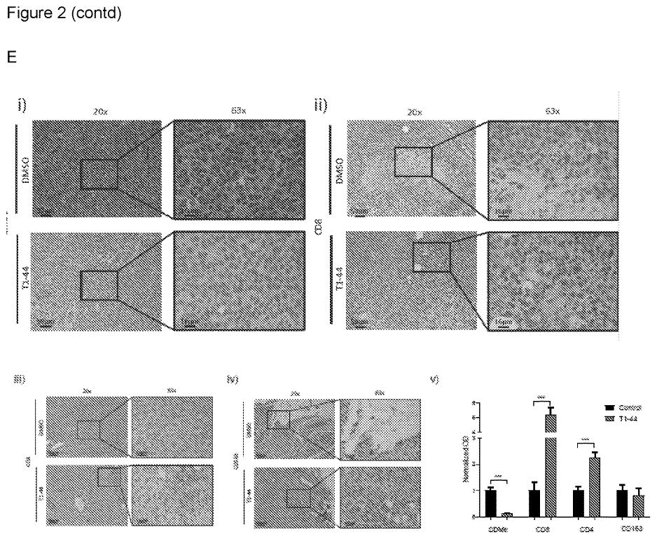

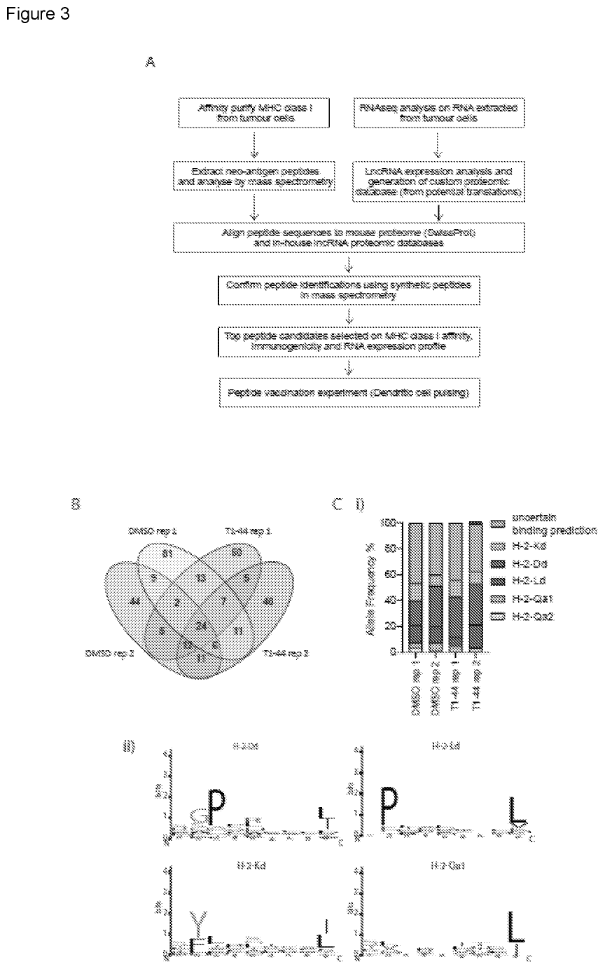

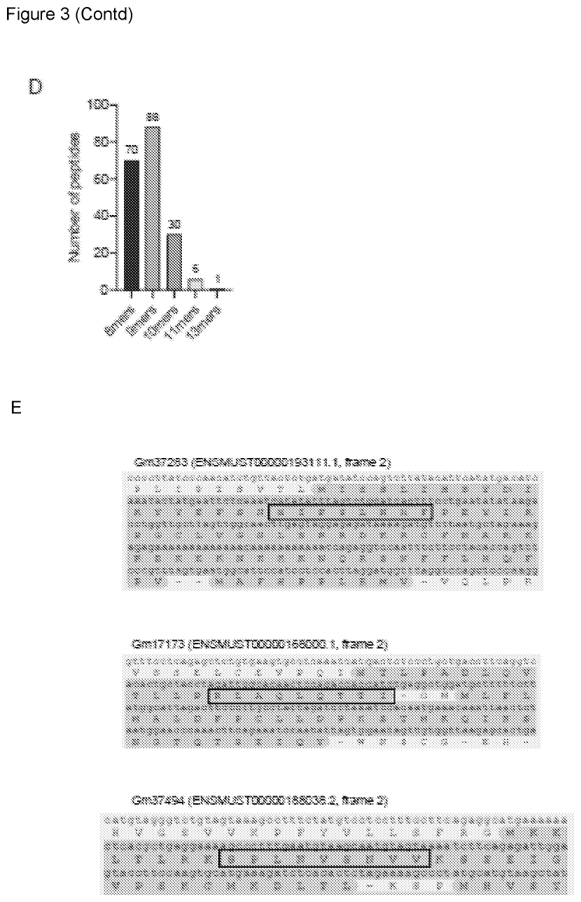

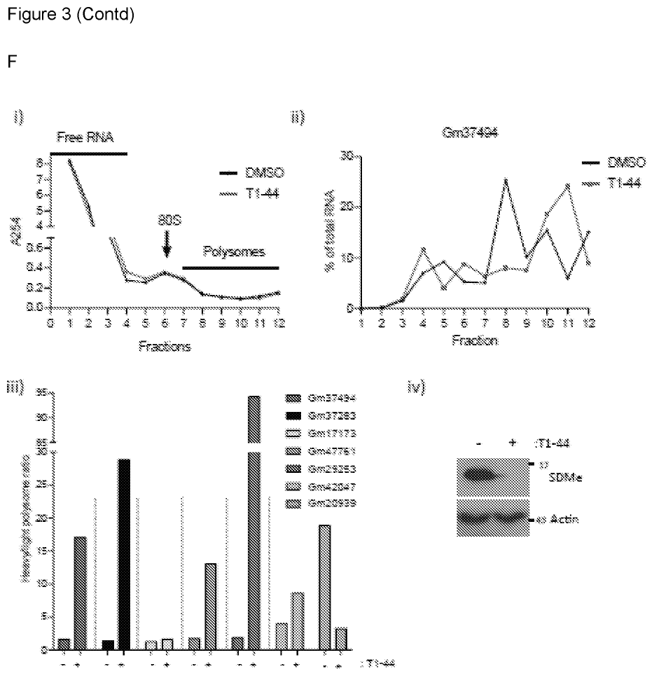

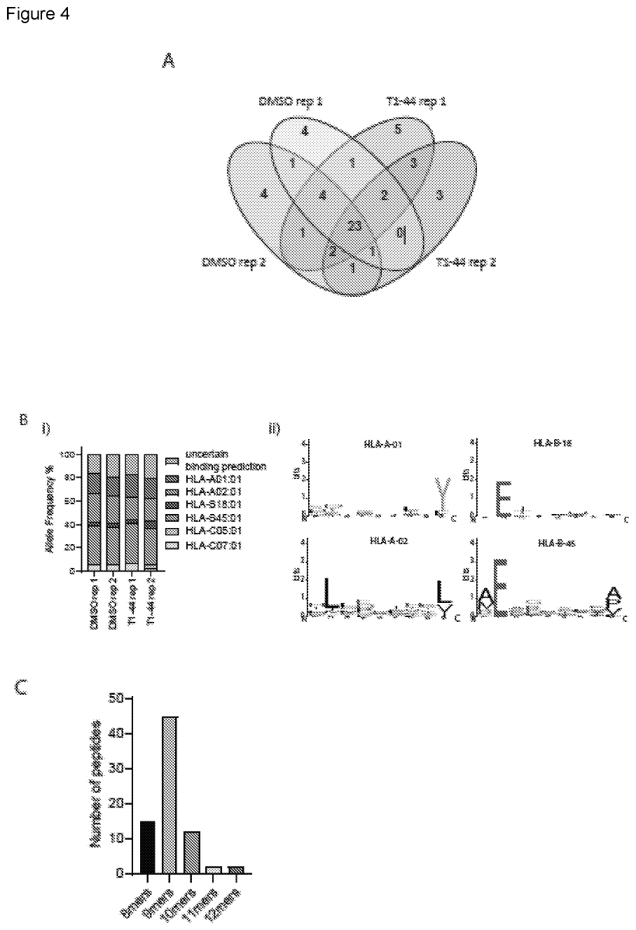

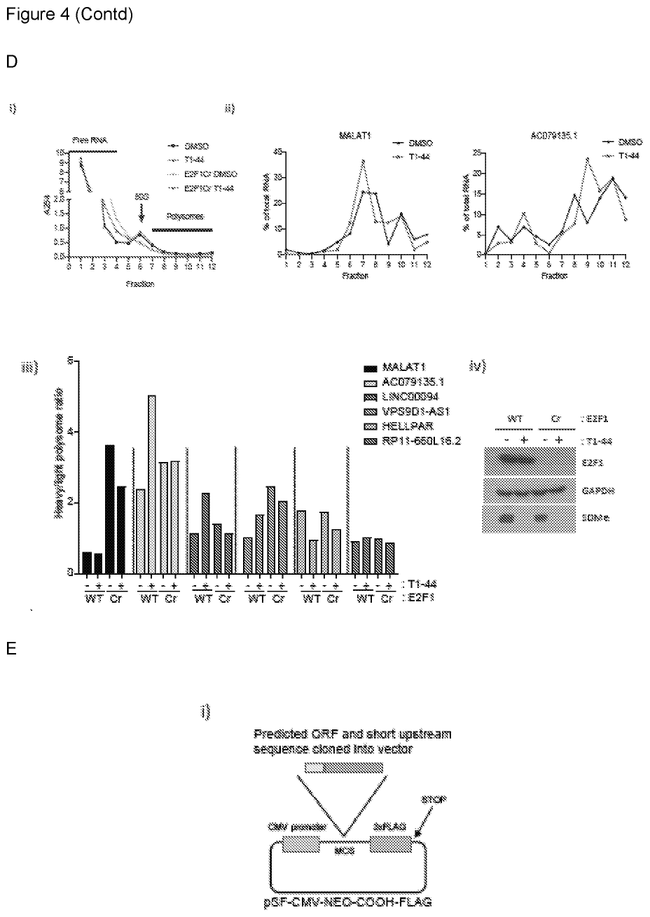

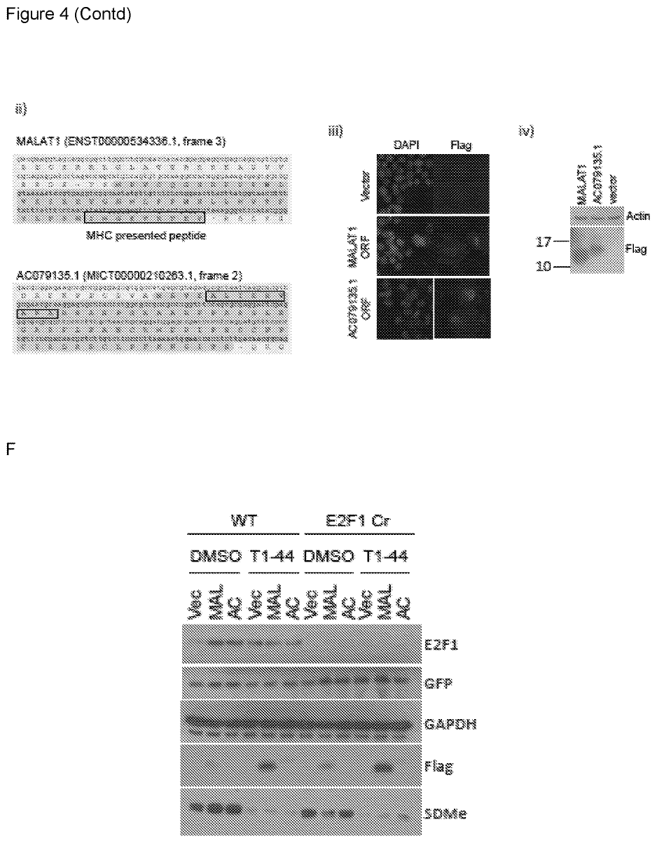

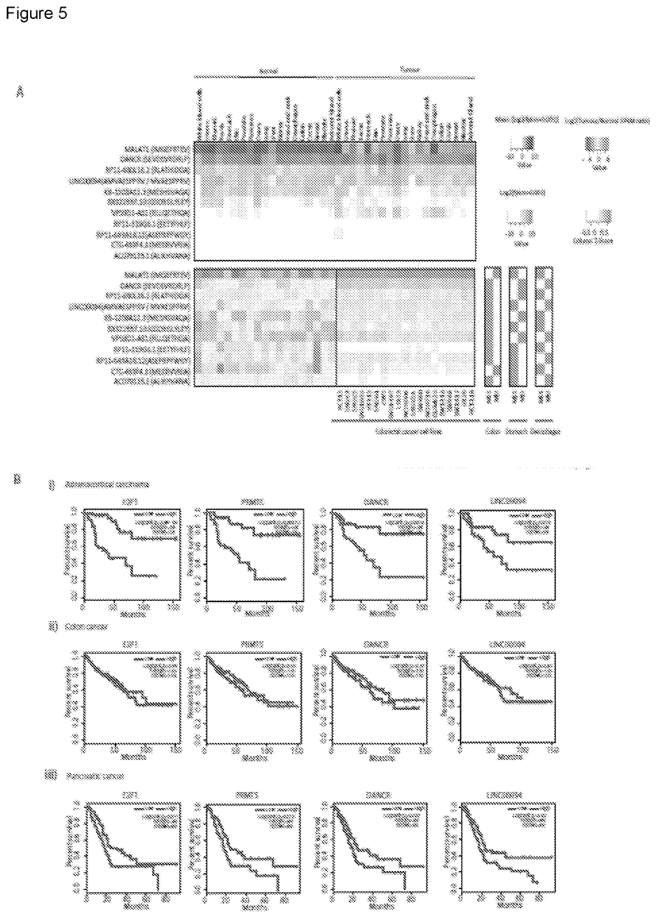

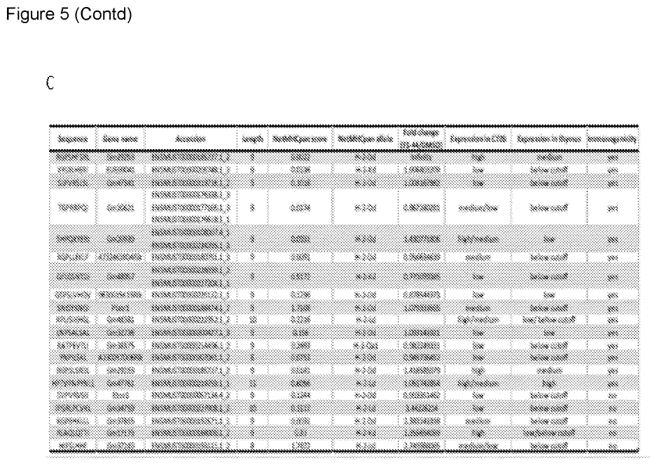

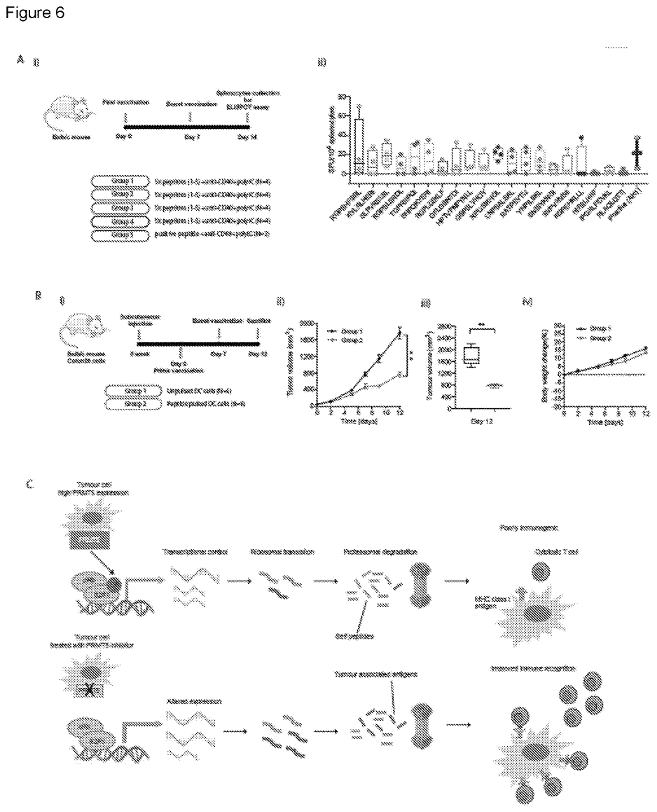

We explored the influence of pharmacological inhibition of PRMT5 activity using a small molecule active site inhibitor, T1-44, and CRISPR knock-out (KO) of the E2F1 gene on expression of lncRNA genes in HCT116 colorectal cancer (CRC) cells (Barczak et al., 2020). RNA-seq datasets were mined for changes in lncRNA gene expression (over 30% change) which revealed that lncRNA transcripts were differentially expressed between each condition. We identified lncRNAs that were either up- or down-regulated (with 237 up-compared to 303 down-regulated), with some overlap of lncRNA expression apparent between the different conditions (FIGS. 1A and B). Differentially expressed lncRNAs represented around 3% of the total lncRNAs present at detectable levels within our RNA-seq datasets (18,377 total lncRNA transcripts). When the expression of a selected group of lncRNA transcripts was individually measured by qPCR, gene expression patterns were apparent that increased upon T1-44 treatment, like LNCOC1, whereas CERNA1, CCNT2-AS1 and UBL7-AS1 decreased (FIG. 1C). Similarly, lncRNA expression was impacted by E2F1 activity; as examples, the LINC01128 and KCTD21-AS1 transcripts were expressed at higher levels in E2F1 KO cells (FIG. 1C).

We selected a group of lncRNA genes to assess whether they are direct targets for E2F1 by inspecting annotated E2F1 ChIP-seq data sets (Dunham et al., 2012). Many of the lncRNA transcripts that scored as differentially expressed upon manipulating PRMT5 and E2F1 were derived from genes that had ChIP-seq reads in close proximity to the transcript start sites (TSS) or within the body of the transcribed sequence. This information prompted us to design primers around the predicted E2F binding sites, which we then used in gene-specific ChIP experiments to confirm the association with E2F1. For example, E2F1 was chromatin-bound in KCTD21-AS1, CERNA1, CCNT2-AS1, and UBL7-AS1 genes in WT E2F1 expressing HCT116 cells, whilst E2F1 KO cells displayed no E2F1 enrichment, as expected (FIG. 1D). Treating cells with T1-44 did not have a significant impact on ChIP bound E2F1 (FIG. 1D).

We evaluated at the genome-wide level the proportion of differentially expressed lncRNAs that harbour E2F binding sites or alternatively are located with close proximity to an established protein coding E2F target gene.

Remarkably, the percentage of lncRNA genes differentially regulated at a statistically significant level (q<0.05) in the RNA-seq dataset that score as potential direct E2F1 target genes (using ChIP-seq data from ENCODE, reads within 500 bp of the TSS) was 39.2%, whilst an additional 38.9% of the lncRNA genes were at the genomic level located close to or within a known E2F target gene (overlapping the gene boundaries of an E2F1 target gene on the same or opposite strand, or contained within the gene boundaries of an E2F1 target gene on the same or opposite strand), though did not appear to be direct E2F targets themselves. 21.9% were non E2F1 target. Thus, the majority of the lncRNA genes identified here have a close relationship with the E2F pathway.

### Example 2

### E2F1. PRMT5 and the Murine Genome

We pursued a genome-wide RNAseq analysis in the murine CRC CT26 cell line. The RNAseq analysis was performed in CT26 cells grown in vitro treated with compound T1-44 which was compared to the control treatment. We mined the RNAseq datasets to evaluate the effect of PRMT5 focusing on lncRNA transcripts. A set of lncRNAs was found to be significantly regulated (at 30% cut-off) with 109 up-regulated and 282 down-regulated upon PRMT5 inhibition relative to the control treatment (FIG. 2A). We confirmed that lncRNA transcripts were regulated when analysed at the single gene level; for example, Gm44148, Gm46565, Ptprv, Epb41l4aos and G630030J09Rik transcripts were differentially expressed upon manipulating PRMT5 (FIG. 2B). Many of the murine lncRNA genes were shown to be E2F1 targets according to E2F1 ChIP-seq data. Remarkably, 83.9% of differentially expressed lncRNAs were derived from genes that contained E2F binding site ChIP-seq peaks with close proximity to their TSS, whilst an additional 8.9% of the lncRNA genes were at the genomic level located close to or within an E2F target gene. We also evaluated the expression in siE2F1 treated cells where the expression level was frequently down-regulated (FIG. 2B), highlighting regulation of murine lncRNA genes by PRMT5 and E2F1.

We followed on with an analysis of lncRNA gene expression in syngeneic colon26 tumours growing in vivo and assessed the impact of T1-44 treatment. Treatment of tumour bearing mice with T1-44 caused a significant delay in tumour growth:

The percentage of lncRNA genes differentially regulated at a statistically significant level (q<0.05) in the CT26 RNA-seq data that score as potential direct E2F1 target genes (using ChIP-seq data from GEO [GSM288349], reads within 500 bp of the TSS) was 83.9%, or whilst not being direct targets themselves 8.9% were found to be associated with other potential E2F1 target genes (overlapping the gene boundaries of an E2F1 target gene on the same or opposite strand, or contained within the gene boundaries of an E2F1 target gene on the same or opposite strand). Non-E2F1 targets represent 7.2%.

RNAseq performed on growing tumours compared to T1-44 treated tumours identified lncRNA transcripts differentially regulated between the two treatment conditions (30% cut-off). At the single lncRNA gene level, 4930473A02Rik, Gm45441, Gm15156, Lncppara, Kcnmb4os1, Lncenc1 and Epb41l4aos transcripts were up-regulated upon treatment with compound T1-44 (FIG. 2C). Moreover, some of the lncRNAs, including Epb41l4aos, Gm44148 and Gm46565 identified in the CT26 RNA-seq followed the same trend in colon26 tumours. We conclude therefore that the PRMT5-E2F1 axis regulates lncRNA gene expression in mouse tumour cells, including tumours growing in situ, in a similar way to that seen in human cancer cell lines.

### Example 3

### PRMT5 Regulates the Immune Response in the Tumour Micro-Environment (TME)

The role of the TME in facilitating an effective anti-tumour response led us to test whether the delayed tumour growth upon PRMT5 inhibition (FIG. 2C) reflected events in the TME caused by the deregulated gene expression. The inhibition of tumour growth upon T1-44 treatment coincided with a reduced level of the SDMe mark within tumour biopsies (FIG. 2E), thus confirming catalytic inhibition of PRMT5. Upon further examination of the TME, we found that T1-44 treatment had a striking impact on the infiltrating lymphocyte population, most clearly evidenced by the influx of cytotoxic CD8 and a modest increase in helper CD4 T lymphocytes (FIG. 2E); on other relevant cell populations, like tumour-associated macrophages, the effect of T1-44 treatment was minimal (FIG. 2E). We considered it possible that the increased level of CD8 T lymphocytes was due to an influence on the adaptive immune response, and because CD8 T lymphocytes principally engage with the MHC class I antigen complex through their T cell receptor, it was plausible that T1-44 treatment influenced antigen presentation via the MHC class I protein complex.

To address whether the peptide antigen content of the MHC class I complex was altered upon treating CT26 cells with compound T1-44, we performed a mass spectrometry (MS) immuno-peptidomics analysis to assess the repertoire of peptides displayed by the MHC class I complex in treated relative to untreated cells (FIGS. 3A and B). The results revealed a large group of MHC class I bound peptides with many derived from cellular proteins. Given the role of the PRMT5-E2F1 axis in regulating lncRNA gene expression, we undertook to examine whether lncRNAs contributed to the MHC class I peptide repertoire. We generated an in-house proteomic database containing predicted translations from all 3-frames of lncRNA transcripts expressed at detectable levels in our CT26 RNA-seq dataset. Peptides detected from the immuno-peptidomics analysis were then matched to either this in-house database or a standard proteomic database containing all reviewed mouse SwissProt protein entries (FIG. 3A). Notably, we identified 382 peptides derived from lncRNA genes (with a mean size of 9 residues), representing 6.5% of the total peptides detected in the analysis (FIG. 3B-D). The lncRNA-derived peptides had predicted high affinity for the murine MHC class I alleles H-2-Kd, Dd, Ld, Qa1, Qa2 and exhibited the conserved residues required for efficient MHC class I binding (FIG. 3C). A selection of the peptide sequences identified in the immuno-peptidomics was subsequently confirmed using mass spectrometry by comparing to the synthetic peptide sequence. Most importantly, both qualitative and quantitative differences were apparent within the population of lncRNA-derived peptides, when the data were compared between T1-44 treated and control cell peptides.

We examined the role of PRMT5 and E2F1 in regulating the peptide coding lncRNAs and found that many of them, like Gm20621, 1110038B12Rik and 4933406J09Rik were up-regulated in CT26 cells grown in vitro and as tumours upon PRMT5 inhibition. Moreover, the expression profile of lncRNAs generally correlated with similar qualitative change in the peptide, such as Gm37283 (peptide sequence HIFSLHHF; SEQ ID NO: 176) and Gm17173 (peptide sequence RLAQLQTTI; SEQ ID NO: 166) which were up-regulated, and 4732463B04Rik (peptide sequence RGPLLEKLF; SEQ ID NO: 167) which was down-regulated upon T1-44 treatment. We also evaluated the impact of E2F1 on the expression of these lncRNAs using siE2F1 silencing and found that of the set tested, lncRNAs were regulated in an E2F1-dependent fashion. In fact, the majority of lncRNA genes that encode MHC class I bound peptides were found to be E2F target genes by reference to ChIP-seq data sets; around 81% were direct E2F1 targets, with a further 8% being associated with or over-lapping other E2F1 target genes.

MHC class I associated peptides are usually generated from larger proteins that are subject to proteolytic degradation and funnelled into the endo-lysosomal vesicular system (Rock et al., 2016). Although lncRNAs are generally regarded to be non-coding (Hartford and Lal, 2020; Statello et al., 2021), because we identified peptides derived from lncRNA genes, we wished to test whether they can encode proteins that could, potentially, be processed to deliver a small peptide. For many of the lncRNAs that gave rise to a peptide, we were able to identify a theoretical open reading frame (ORF) from which the peptide would have been derived (FIG. 3E); most of the ORFs were small encoding polypeptides with less than 100 residues although another group of lncRNAs was apparent that had larger ORFs. Further, transcripts derived from the lncRNA genes were able to associate with the translating polysomal fraction of ribosomes, including for example Gm 37494, Gm37283, and Gm17173 (FIG. 3E). In some cases, like Gm37283 and Gm47761, their association was enhanced upon PRMT5 inhibition (FIG. 3Fiii) which in turn coincided with increased levels of the derived peptide (HIFSLHHF; SEQ ID NO: 176 and YYIPGLKGI; SEQ ID NO: 194, respectively).

We subsequently tested whether lncRNA gene RNA can be directly translated into a detectable protein which we performed by cloning the predicted ORF cDNA into an expression vector tagged with a FLAG epitope at the C-terminal end. As an example, the Gm29253 lncRNA had a theoretical ORF encoding a polypeptide of 26 kD. A specific polypeptide derived from ectopic expression of the Gm29253 ORF was detected in transfected cells by immuno-staining and immunoblotting, with the anticipated molecular weight for the predicted ORF. We therefore conclude that lncRNAs that give rise to MHC class I bound peptides can associate with ribosomes and further be translated into polypeptides which subsequently are processed to deliver peptides to the MHC class I protein complex.

### Example 4

### LncRNA-Derived MHC Class I Associated Peptides in Human Tumour Cells

We performed a similar immuno-peptidomics analysis on MHC class I associated peptides in human HCT116 cells comparing untreated with T1-44 treated cells, which also highlighted a significant set of peptides derived from human lncRNA genes (118 unique peptides identified from all in-house databases; FIG. 4A-C; the additional larger set derived from cellular proteins was also apparent. Individual lncRNA peptide sequences were confirmed by comparing the mass spectrometry derived peptide sequence to its synthetic peptide counterpart. The size of the peptides averaged at 9 residues with the anticipated conserved residues required for human HLA MHC class I binding (FIGS. 4B and C) and were predicted to have high affinity for the human HLA-A, -B, and -C allele MHC class 1 proteins (FIG. 4B). Furthermore, quantitative analysis indicated that 42% of the peptides (as identified from the lncRNA database using GENCODE annotation) were regulated upon T1-44 treatment with a 30% change or over, where 10% were upregulated, 32% downregulated and 58% unchanged.

We measured the expression of human lncRNAs that give rise to MHC class I bound peptides by qPCR and found that upon T1-44 treatment many were differentially expressed, and some of the expression changes reflected the similar relative changes seen in the MHC class I associated peptide. For example, the increased level of peptides derived from HELLPAR (peptide sequence LSLSLSLQFS; SEQ ID NO: 127) and RP11-660L16.2 (peptide sequence RLATHIDGA; SEQ ID NO: 36) lncRNAs reflected increased lncRNA expression under T1-44 treatment, whilst a number which displayed reduced expression including AC079135.1 (peptide sequence AEKPPGSVA; SEQ ID NO: 106), RP11-319G6.1 (peptide sequence EETYFHLF; SEQ ID NO: 20) and VPS9D1-AS1 (peptide sequence RLLQETHQA; SEQ ID NO: 38) lncRNAs coincided with reduced levels of peptide bound to MHC class I antigen. The expression of many of the lncRNAs were also impacted by E2F1, displaying either increased (AC004943.2, PPM1F-AS1, AC018445.6) or decreased expression (C5orf34-AS1, RP11-319G6.1, AC079135.1) in the E2F1 KO cell line.

We confirmed that in human cancer cells, many of the peptide-encoding lncRNAs represent E2F1-target genes. We used ChIPseq data to identify E2F1 binding sites, designed primers surrounding these sites and then by ChIP confirmed the presence of chromatin-bound E2F1. E2F1 was observed to be enriched at the promoters of many of the lncRNA genes that produced peptides; around 39% and 77% of lncRNAs from GENCODE and FANTOM databases, respectively, appeared to be direct E2F1 target genes, whilst a further 26% (GENCODE) and 14% (FANTOM) of peptide-encoding lncRNA genes were associated with other E2F1 target genes respectively. The impact of T1-44 treatment on E2F1 recruitment appeared to be modest.

We performed a similar analysis on the ORFs within the human lncRNAs expressed in HCT116 cells. Most of the lncRNA ORFs that gave rise to the MHC bound peptides were less than 100 residues, producing a polypeptide of less than 12 kD, and a weak translation initiating sequence was apparent when all the ORFs were compared. We tested whether the lncRNAs could associate with the translating polysomal fraction of ribosomes and found that many lncRNAs, for example MALAT1, AC079135.1, and VPS9D1-AS1, were able to do so (FIG. 4D; in some cases, there was a sedimentation shift upon PRMT5 inhibition and E2F1 KO conditions, for example MALAT1 and VPS9D1-AS1 (FIG. 4D).

Following on, we addressed whether human lncRNA transcripts can be directly translated into protein by cloning the predicted ORF as a cDNA into an expression vector. For the two examples tested, MALAT1 and A0079135.1, small proteins derived from the ORF containing the MHC associated peptide could be detected by immuno-staining and immunoblotting (FIGS. 4E and 4F). In addition, the expression of these lncRNA polypeptides was induced after treatment with PRMT5 inhibitors, whilst the expression of GFP from a control plasmid was not affected, suggesting that the translation of lncRNA derived polypeptides can be further regulated by PRMT5 (FIG. 4F).

### Example 5

### Relevance to Human Cancer

It is noteworthy that some of the PRMT5-E2F1 responsive lncRNAs which encode MHC bound peptides, like MALAT1 and DANCR, are already known to exhibit deregulated expression in human cancer (Zhao et al., 2021). We therefore evaluated some of the less characterised peptide-encoding lncRNAs (identified here) for expression patterns in malignant disease. As part of this exercise, we confirmed MALAT1 and DANCR expression across a range of cancers and normal tissue (FIG. 5A). The expression pattern of other lncRNAs was variable; for example, VPS9D1-AS1 exhibited heterogenous expression, with high expression in some cancers and generally low expression in normal tissue. This contrasted with CTC-459F4 which had uniformly low expression in cancer and normal tissue.

In a detailed analysis of expression in CRC cell lines, lncRNA expression was detected across a range of tumour cell lines, with some lncRNA genes exhibiting high and others lower expression on the panel of cell lines (FIG. 5A). Interestingly, when lncRNA expression was analysed in RNA prepared from human colorectal, stomach and oesophageal cancer, there was clear differentiation between expression in the micro-satellite stable (MSS) and micro-satellite instable (MSI) sub-groups; for example, in colorectal cancer the majority of lncRNA expression occurred in the MSS sub-group and not the MSI sub-group, which was less marked in stomach and oesophageal cancers (FIG. 5A). Generally, therefore, the expression of the lncRNAs is disease-specific.

It was of further interest to assess the prognostic significance of the lncRNAs in human disease, where we focused on adrenocortical (AC) carcinoma, CRC and pancreatic cancer (PC), and measured the expression level across a collection of tumour biopsies and related this information to survival. PRMT5 levels were of prognostic significance in AC as high levels correlated with poor prognosis; E2F1 expression followed a similar trend (FIG. 5Bi). Some lncRNAs behaved in a similar way, with high expression correlating with poor prognosis (for example DANCR and LINC00094; FIG. 5Bi). However, this relationship was less apparent in CRC and PC (FIGS. 5Bii and 5Biii). Generally, it appears that the prognostic value of lncRNA expression is quite good in AC, but quite variable in CRC and PC.

### Example 6

Immunogenicity and Tumour Growth Inhibition with lncRNA Derived Peptides

We tested whether the lncRNA derived MHC class I bound peptides were immunogenic in mice. Twenty peptides encoded by murine lncRNA genes and identified in the immuno-peptidomics analysis were chosen for the immunological analysis based on their predicted high affinity for MHC class I, low expression of their counterpart lncRNA gene in normal mouse tissue and differential regulation upon PRMT5 inhibition (FIG. 5C). Pools of five peptides were used to immunise mice when, at day 14, splenocytes were harvested for IFN y ELISpot by restimulating the cells from each mouse with the relevant pool of peptides. Out of the peptides used to immunize the mice, we identified 15 peptides which were immunogenic, resulting in a significant increase in IFN y activated T cells, compared to unimmunized mice (FIG. 6A). We included a positive immunogenic control peptide (AHI1) and in addition several lncRNA derived peptides which failed to exhibit immunogenicity (FIG. 6A). These results provide evidence that lncRNA-derived MHC class I bound peptides are immunogenic and stimulate an effective T cell response.

We were interested to test whether the T cell response against the lncRNA-derived peptides would translate into a therapeutic benefit when they were delivered in the context of a cancer vaccine, namely whether they augment the anti-tumour response. The approach that we took to address this question was to use an ex vivo dendritic cell delivery platform. Bone marrow dendritic cells were harvested from mice, matured and then pulsed with the pooled immunogenic peptides. After 7 days, the peptide pulsed and control dendritic cells were introduced into Balb/c mice with established syngeneic colon26 tumours, and any effect on tumour growth monitored. Strikingly, transfer of the peptide pulsed dendritic cells enhanced the anti-tumour response, reflected as delayed growth of the colon26 tumours compared to the control treated dendritic cells (FIG. 6B). In the context of dendritic cell delivery, lncRNA derived peptides are able to hinder the growth of colon26 tumours (from which the peptides were initially derived) and thus provide a therapeutic benefit.

### Discussion

The classical view of the pRb-E2F pathway as a regulator of cell growth and division through control of G1 to S phase transcription has advanced considerably in recent years. It is now clear that a much wider repertoire of target genes is subject to E2F pathway control, which includes genes where the effect of E2F is atypical, affecting alternative splicing rather than the usual transcriptional effect (Roworth et al., 2019). Arginine methylation mediated by PRMT5, which targets a small R-rich region in E2F1, is a major influence on gene expression control by E2F1 by determining which mechanism of gene control predominates (Zheng et al., 2013). Given the frequent over-expression of PRMT5 in cancer (Bedford and Clarke, 2009; Bedford and Richard, 2005; Jansson et al., 2008; Yang and Bedford, 2013), its role in regulating E2F activity is likely to be highly important. In this study, we have described an unexpected set of observations which extend the influence of the PRMT5-E2F1 axis to control of lncRNA genes and further to a role in immune recognition. A key finding of our study shows that a large number of lncRNA genes, directly under E2F pathway control, encode small peptides that assemble with MHC class I proteins and are subsequently recognised by the immune system.

Human genomic analysis has suggested that there are more than 17,000 lncRNA genes (GENCODE v38) (Statello et al., 2021), with others implying around 28,000 human lncRNAs genes (FANTOM5 database) (Hon et al., 2017). LncRNAs are usually over 200 nucleotides in length, and a small number have been described to be processed and spliced in a similar way to mRNAs (Statello et al., 2021). Whether lncRNAs are biologically important remains a debated topic; some have been ascribed cellular functions, for example, in chromatin biology (Schlackow et al., 2017; Vos et al., 2018). In other studies, lncRNAs may influence RNA biogenesis (Statello et al., 2021). Further, lncRNA expression has been connected with cancer. For example, MALAT1 is a highly conserved lncRNA that is abundantly expressed in cells and tissues and may play a role in regulating genes at both the transcriptional and post-transcriptional levels (Engreitz et al., 2014; Yang et al., 2013). MALAT1 was initially identified as exhibiting elevated expression in metastatic lung cancer and the same has been described in different solid or lymphoid tumours. In a murine metastatic cancer model, MALAT1 loss resulted in differentiation of primary tumours and a significant reduction in metastasis (Arun et al., 2016).

Through a detailed immuno-peptidomic analysis of the peptide composition of MHC class I proteins on cancer cells (human and mouse), we identified a significant proportion of peptides derived from lncRNA genes that were in turn shown to be genes regulated by the PRMT5-E2F1 axis. Many of these peptides were regulated upon pharmacological inhibition of PRMT5 activity in addition to being E2F1 target genes, indicating that together PRMT5 and E2F1 regulate the immunogenicity of cancer cells and therefore in turn the immune response against tumours. Interestingly, MALAT1 was one lncRNA that we found to encode a peptide derived from a larger polypeptide encoded by an ORF in the coding sequence. It is noteworthy that bioinformatics approaches have been used to suggest that tumour associated antigens can be derived from non-canonical parts of the genome, although the biological basis and therapeutic significance remains for the most part to be elucidated (Chong et al., 2020; Laumont et al, 2018; Zhang et al, 2018). The results described here firmly establish a new and important role for lncRNA genes in contributing to the peptide composition of MHC class I antigen presentation machinery.

lncRNA genes may be aberrantly expressed in cancer, exhibiting either increased or decreased expression (Huarte, 2015). In the context of the results described here, the altered expression level could translate into quantitative differences in the spectrum of peptides presented to the immune system by cancer cells and thereby impact on immune recognition. These observations in turn shed light on a cancer-relevant pathway regulated by PRMT5; namely, the increased level of PRMT5 widely reported in many human cancers through control of lncRNA expression impacts on the immunogenicity of tumour cells, by altering the peptide repertoire presented to the immune system. It is noteworthy that the peptides derived from lncRNA genes are self-antigens and therefore, theoretically, T cells directed against such peptide antigens should be eliminated during development by central and peripheral tolerance mechanisms. However, we found that many of the peptides tested could drive an active T cell response, suggesting that any tolerance which does exist against this class of lncRNA derived peptides is either partial or can be over-ridden. The fact that we gained evidence for a T cell response against the synthetic lncRNA-derived peptides led us on to test whether this would translate into an anti-cancer response against tumours. For this, we used the colon26 model, from where the peptides were originally identified and importantly are naturally expressed and presented in the context of MHC class I proteins. Our results suggested that this is indeed the case. Thus, in the colon26 syngeneic mouse model, lncRNA derived peptides delivered through ex vivo peptide loaded dendritic cells could stimulate an effective anti-cancer immune response.

The results highlight lncRNA genes as an unanticipated rich source of tumour associated antigens, controlled by the PRMT5-E2F1 axis and presented to the immune system through the classical route as MHC class I associated antigens (FIG. 6C). The ability to influence their expression through pharmacological manipulation of PRMT5, itself an enzyme with high cancer-relevance, allows for a new approach to control the immunogenicity of tumour cells and further develop cancer vaccines that can ultimately be aligned to a specific type of cancer. Our study also integrates the pRb-E2F pathway, which is a fundamental regulator of cell cycle progression and often de-regulated in cancer, with antigen presentation and the immune response and thus establishes the interplay between cancer cell proliferation and the immune system.

### REFERENCES

Arun, G., Diermeier, S., Akerman, M., Chang, K. C., Wilkinson, J. E., Hearn, S., Kim, Y., MacLeod, A. R., Krainer, A. R., Norton, L., et al. (2016). Differentiation of mammary tumors and reduction in metastasis upon Malat1 lncRNA loss. Gene Dev 30, 34-51.
Barczak, W., Jin, L., Carr, S. M., Munro, S., Ward, S., Kanapin, A., Samsonova, A., and La Thangue, N. B. (2020). PRMT5 promotes cancer cell migration and invasion through the E2F pathway. Cell Death Dis 11.
Bedford, M. T., and Clarke, S. G. (2009). Protein Arginine Methylation in Mammals: Who, What, and Why. Mol Cell 33, 1-13.
Bedford, M. T., and Richard, S. (2005). Arginine methylation: An emerging regulator of protein function. Mol Cell 18, 263-272.
Blanchet, E., Annicotte, J. S., Lagarrigue, S., Aguilar, V., Clape, C., Chavey, C., Fritz, V., Casas, F., Apparailly, F., Auwerx, J., and Fajas, L. (2011). E2F transcription factor-1 regulates oxidative metabolism. Nat Cell Biol 13, 1146-U1184.
Carr, S. M., Munro, S., Sagum, C. A., Fedorov, O., Bedford, M. T., and La Thangue, N. B. (2017). Tudor-domain protein PHF20L1 reads lysine methylated retinoblastoma tumour suppressor protein. Cell Death Differ 24, 2139-2149.
Cho, E. C., Zheng, S., Munro, S., Liu, G., Carr, S. M., Moehlenbrink, J., Lu, Y. C., Stimson, L., Khan, O., Konietzny, R., et al. (2012). Arginine methylation controls growth regulation by E2F-1. EMBO J 31, 1785-1797.
Chong, C., Muller, M., Pak, H., Harnett, D., Huber, F., Grun, D., Leleu, M., Auger, A., Arnaud, M., Stevenson, B. J., et al. (2020). Integrated proteogenomic deep sequencing and analytics accurately identify non-canonical peptides in tumor immunopeptidomes. Nat Commun 11, 1293.
DeFreitas, R. F., Ivanochko, D., and Schapira, M. (2019). Methyltransferase Inhibitors: Competing with, or Exploiting the Bound Cofactor. Molecules 24, 4492.
Denchi, E. L., and Helin, K. (2005). E2F1 is crucial for E2F-dependent apoptosis. Embo Rep 6, 661-667.
Dunham, I., Kundaje, A., Aldred, S. F., Collins, P. J., Davis, C., Doyle, F., Epstein, C. B., Frietze, S., Harrow, J., Kaul, R., et al. (2012). An integrated encyclopedia of DNA elements in the human genome. Nature 489, 57-74.
Engreitz, J. M., Sirokman, K., McDonel, P., Shishkin, A. A., Surka, C., Russell, P., Grossman, S. R., Chow, A. Y., Guttman, M., and Lander, E. S. (2014). RNA-RNA Interactions Enable Specific Targeting of Noncoding RNAs to Nascent Pre-mRNAs and Chromatin Sites. Cell 159, 188-199.
Gebert, L. F. R., and MacRae, I. J. (2019). Regulation of microRNA function in animals. Nat Rev Mol Cell Bio 20, 21-37.
Gutschner, T., Hammerle, M., Eissmann, M., Hsu, J., Kim, Y., Hung, G., Revenko, A., Arun, G., Stentrup, M., Gross, M., et al. (2013). The Noncoding RNA MALAT1 Is a Critical Regulator of the Metastasis Phenotype of Lung Cancer Cells. Cancer Res 73, 1180-1189.
Hartford, C. C. R., and Lal, A. (2020). When Long Noncoding Becomes Protein Coding. Mol Cell Biol 40.
Hon, C. C., Ramilowski, J. A., Harshbarger, J., Bertin, N., Rackham, O. J. L., Gough, J., Denisenko, E., Schmeier, S., Poulsen, T. M., Severin, J., et al. (2017). An atlas of human long non-coding RNAs with accurate 5′ ends. Nature 543, 199-+.
Huang, A. Y. C., Gulden, P. H., Woods, A. S., Thomas, M. C., Tong, C. D., Wang, W., Engelhard, V. H., Pasternack, G., Cotter, R., Hunt, D., et al. (1996). The immunodominant major histocompatibility complex class I-restricted antigen of a murine colon tumor derives from an endogenous retroviral gene product. P Natl Acad Sci USA 93, 9730-9735.
Huarte, M. (2015). The emerging role of lncRNAs in cancer. Nat Med 21, 1253-1261. Jansson, M., Durant, S. T., Cho, E. C., Sheahan, S., Edelmann, M., Kessler, B., and La Thangue, N. B. (2008). Arginine methylation regulates the p53 response. Nat Cell Biol 10, 1431-U1122.
Ji, P., Diederichs, S., Wang, W., Boing, S., Metzger, R., Schneider, P. M., Tidow, N., Brandt, B., Buerger, H., Bulk, E., et al. (2003). MALAT-1, a novel noncoding RNA, and thymosin beta4 predict metastasis and survival in early-stage non-small cell lung cancer. Oncogene 22, 8031-8041.
Kent, L. N., and Leone, G. (2019). The broken cycle: E2F dysfunction in cancer. Nat Rev Cancer 19, 326-338.
Kozak, M. (1987). An Analysis of 5′-Noncoding Sequences from 699 Vertebrate Messenger-Rnas. Nucleic Acids Res 15, 8125-8148.
Laumont, C. M., Vincent, K., Hesnard, L., Audemard, E., Bonneil, E., Laverdure, J. P., Gendron, P., Courcelles, M., Hardy, M. P., Cote, C., et al. (2018). Noncoding regions are the main source of targetable tumor-specific antigens. Sci Transl Med 10.
Munro, S., Carr, S. M., and La Thangue, N. B. (2012). Diversity within the pRb pathway: is there a code of conduct? Oncogene 31, 4343-4352.
Rock, K. L., Reits, E., and Neefjes, J. (2016). Present Yourself! By MHC Class I and MHC Class II Molecules. Trends Immunol 37, 724-737.
Paston, S. j., Brentville, V. A., Symonds, P., Durrant, L. G. Cancer vaccines, Adjuvants and Delivery Systems. Frontiers in Immunology. 12:1-21, article 627932, 2021.
Roworth, A. P., Carr, S. M., Liu, G., Barczak, W., Miller, R. L., Munro, S., Kanapin, A., Samsonova, A., and La Thangue, N. B. (2019). Arginine methylation expands the regulatory mechanisms and extends the genomic landscape under E2F control. Sci Adv 5.
Roworth, A. P., Ghari, F., and La Thangue, N. B. (2015). To live or let die—complexity within the E2F1 pathway. Mol Cell Oncol 2.
Schlackow, M., Nojima, T., Gomes, T., Dhir, A., Carmo-Fonseca, M., and Proudfoot, N. J. (2017). Distinctive Patterns of Transcription and RNA Processing for Human lincRNAs. Mol Cell 65, 25-38.
Statello, L., Guo, C. J., Chen, L. L., and Huarte, M. (2021). Gene regulation by long non-coding RNAs and its biological functions (vol 22, pg 96, 2021). Nat Rev Mol Cell Bio 22, 159-159.
Vos, S. M., Farnung, L., Boehning, M., Wigge, C., Linden, A., Urlaub, H., and Cramer, P. (2018). Structure of activated transcription complex Pol II-DSIF-PAF-SPT6. Nature 560, 607-+.
Wang, Z. W., Katsaros, D., Biglia, N., Shen, Y., Fu, Y. Y., Loo, L. W. M., Jia, W., Obata, Y., and Yu, H. (2018). High expression of long non-coding RNA MALAT1 in breast cancer is associated with poor relapse-free survival. Breast Cancer Res Tr 171, 261-271.
Wu, L. Z., Timmers, C., Maiti, B., Saavedra, H. I., Sang, L., Chong, G. T., Nuckolls, F., Giangrande, P., Wright, F. A., Field, S. J., et al. (2001). The E2F1-3 transcription factors are essential for cellular proliferation. Nature 414, 457-462.
Yang, L. Q., Lin, C. R., Liu, W., Zhang, J., Ohgi, K. A., Grinstein, J. D., Dorrestein, P. C., and Rosenfeld, M. G. (2013). ncRNA- and Pc2 Methylation-Dependent Gene Relocation between Nuclear Structures Mediates Gene Activation Programs (vol 147, pg 773, 2011). Cell 155, 478-478.
Yang, Y. Z., and Bedford, M. T. (2013). Protein arginine methyltransferases and cancer. Nat Rev Cancer 13, 37-50.
Yao, G., Lee, T. J., Mori, S., Nevins, J. R., and You, L. C. (2008). A bistable Rb-E2F switch underlies the restriction point. Nat Cell Biol 10, 476-U255.
Zhang, M. Y., Fritsche, J., Roszik, J., Williams, L. J., Peng, X. X., Chiu, Y. L., Tsou, C. C., Hoffgaard, F., Goldfinger, V., Schoor, O., et al. (2018). RNA editing derived epitopes function as cancer antigens to elicit immune responses. Nature Communications 9.
Zhao, S., Zhang, X., Chen, S., and Zhang, S. (2021). Long noncoding RNAs: fine-tuners hidden in the cancer signaling network. Cell Death Discov 7, 283.
Zheng, S., Moehlenbrink, J., Lu, Y. C., Zalmas, L. P., Sagum, C. A., Carr, S., McGouran, J. F., Alexander, L., Fedorov, O., Munro, S., et al. (2013). Arginine methylation-dependent reader-writer interplay governs growth control by E2F-1. Mol Cell 52, 37-51.

1. A peptide derived from a PRMT5-E2F1 axis regulated long non-coding RNA gene or a derivative thereof.

2. The peptide according to claim 1, wherein said peptide is transcribed from a lncRNA gene sequence by a promoter with an E2F binding site.

3. The peptide according to claim 1 or 2, which is immunogenic.

4. The peptide according to claim 1, 2 or 3, which is regulated in a cell when PRMT5 is inhibited.

5. The peptide according to claim 4, which is upregulated in a cell when PRMT5 is inhibited.

6. The peptide according to any one of claim 1-5, wherein said peptide is capable of inducing an immune response.

7. A nucleic acid, encoding a peptide according to any one of claims 1 to 6.

8. The nucleic acid according to claim 7 which is DNA, cDNA, PNA, RNA or combinations thereof.

9. An expression vector capable of expressing a nucleic acid according to claim 7 or 8.

10. A host cell comprising a nucleic acid according to claim 7 or 8 or an expression vector according to claim 9.

11. The host cell according to claim 10, selected from the group consisting of a bacterial, fungal (including yeast), and mammalian cell.

12. A vaccine comprising at least one peptide according to any one of claims 1 to 6 or a nucleic acid encoding said peptide.

13. The vaccine according to claim 12, wherein the vaccine comprises at least 5 lncRNA gene peptides or nucleic acid encoding such peptides.

14. The vaccine according to claim 12 or 13, wherein the vaccine is a peptide vaccine, RNA vaccine, DNA vaccine, viral vector vaccine or dendritic cell vaccine.

15. The vaccine according to claim 14, wherein the viral vector is derived from adenovirus, adeno-associated virus (AAV), herpesvirus, poxvirus, alpha virus (such as Semliki Forest virus, Sindbis virus, or Venezuelan equine encephalitis virus), arenaviruses (like lassa virus, machupovirus, or junonvirus), measles virus, vaccinia virus, retrovirus (including lentivirus), or influenza virus

16. The vaccine according to claim, wherein the vector is a simian adenovirus vaccine such as ChAdOx vector.

17. The vaccine according to any one of claims 12 to 16, wherein the immunogenic peptide is presented by a human leukocyte antigen (HLA) class I molecule, including one selected from the group comprising: HLA A, HLA A2 or HLA 24 or HLA AI or HLA A3.

18. The vaccine according to any one of claims 12 to 17, comprising multiple lncRNA encoded peptides that are overexpressed by the cancer cell when PRMT5 is inhibited within a chimeric polypeptide.

19. The vaccine according to claim 18, wherein the multiple lncRNA encoded peptides are linked together in series.

20. The vaccine according to claim 18 or 19, wherein the multiple peptides are encoded by a single nucleic acid molecule.

21. The vaccine according to any one of claims 12 to 20, which stimulates an immune reaction in the host receiving the vaccine.

22. The vaccine according to claim 21, wherein the host immune reaction is a humoral response or adaptive cell mediated immune response.

23. The vaccine according to any one of claims 12 to 22, which when administered to a subject stimulates the production of anti-cancer CD-8 T-cells.

24. A method for making a tumour specific vaccine, comprising identifying peptides expressed by the tumour that are encoded by a PRMT5-E2F1 axis regulated long non-coding RNA gene; incorporating one or more of said peptides or a nucleic acid encoding said one or more peptide(s) into a vaccine.

25. A pharmaceutical composition comprising the peptide of any one of claims 1 to 6, the nucleic acid of claim 7 or 8, or the vaccine of any one of claims 12-23, and a pharmaceutically acceptable excipient.

26. The peptide of any one of claims 1 to 6, the nucleic acid of claim 7 or 8, the vaccine of any one of claims 12-23 or the pharmaceutical composition of claim 25 for use in therapy.

27. The peptide of any one of claims 1 to 6, the nucleic acid of claim 7 or 8, the vaccine of any one of claims 12-23 or the pharmaceutical composition of claim 25 for use in treating cancer.

28. The peptide of any one of claims 1 to 6, the nucleic acid of claim 7 or 8, the vaccine of any one of claims 12-23 or the pharmaceutical composition of claim 25 for use in treating cancer in combination with a PRMT5 inhibitor.

29. A method of treating a subject having, or suspected of having cancer, comprising administering the peptide of any one of claims 1 to 6, the nucleic acid of claim 7 or 8, the vaccine of any one of claims 12-23 or the pharmaceutical composition of claim 25 to said subject.

30. The peptide of any one of claims 1 to 6, the nucleic acid of claim 7 or 8, the vaccine of any one of claims 12-23 or the pharmaceutical composition of claim 25 for use in treating cancer, wherein the cancer is selected from the group consisting of: leukaemia, lymphoma, multiple myeloma, lung cancer, liver cancer, breast cancer, head and neck cancer, neuroblastoma, thyroid carcinoma, skin cancer (including melanoma), oral squamous cell carcinoma, urinary bladder cancer, Leydig cell tumour, biliary cancer, such as cholangiocarcinoma or bile duct cancer, pancreatic cancer, colon cancer, colorectal cancer and gynaecological cancers, including ovarian cancer, endometrial cancer, fallopian tube cancer, uterine cancer and cervical cancer, including epithelia cervix carcinoma, optionally, the cancer is leukaemia selected from the group consisting of acute lymphoblastic leukaemia, acute myelogenous leukaemia (also known as acute myeloid leukaemia or acute non-lymphocytic leukaemia), acute promyelocytic leukaemia, acute lymphocytic leukaemia, chronic myelogenous leukaemia (also known as chronic myeloid leukaemia, chronic myelocytic leukaemia or chronic granulocytic leukaemia), chronic lymphocytic leukaemia, monoblastic leukaemia and hairy cell leukaemia.

31. A method for selecting peptides for inclusion in a tumour vaccine, comprising (i) contacting a tumour cell sample from a patient with a PRMT5 inhibitor; (ii) determining the expression level of one or more lncRNA gene encoded peptides by the contacted cells and (iii) selecting one or more peptides that are upregulated or downregulated for inclusion in a tumour vaccine.

32. A method for selecting peptides for inclusion in a tumour vaccine, comprising contacting cells of a tumour type with a PRMT5 inhibitor and determining the expression level of one or more lncRNA gene encoded peptides by the contacted cells and selecting one or more peptides that are upregulated or downregulated for inclusion in a tumour vaccine.

33. The method of claim 31 or 32, wherein prior to step (iii) peptides which are upregulated or downregulated are assessed to determine whether they are presented or likely to be presented on cell surfaces via MHC, such as MHC class I.

34. The method of claim 31, 32 or 33, wherein the tumour is selected from: leukaemia, lymphoma, multiple myeloma, lung cancer, liver cancer, breast cancer, head and neck cancer, neuroblastoma, thyroid carcinoma, skin cancer (including melanoma), oral squamous cell carcinoma, urinary bladder cancer, Leydig cell tumour, biliary cancer, such as cholangiocarcinoma or bile duct cancer, pancreatic cancer, colon cancer, colorectal cancer and gynaecological cancers, including ovarian cancer, endometrial cancer, fallopian tube cancer, uterine cancer and cervical cancer, including epithelia cervix carcinoma, optionally, the cancer is leukaemia selected from the group consisting of acute lymphoblastic leukaemia, acute myelogenous leukaemia (also known as acute myeloid leukaemia or acute non-lymphocytic leukaemia), acute promyelocytic leukaemia, acute lymphocytic leukaemia, chronic myelogenous leukaemia (also known as chronic myeloid leukaemia, chronic myelocytic leukaemia or chronic granulocytic leukaemia), chronic lymphocytic leukaemia, monoblastic leukaemia and hairy cell leukaemia.

35. A PRMT5 inhibitor for use in the treatment of cancer by stimulating a host immune response, such as a humoral response or cell-mediated immune response.

36. The PRMT5 inhibitor for use according to claim 35, wherein the PRMT5 inhibitor is selected from the group consisting of: an antibody, an siRNA, an antisense oligonucleotide (ASO) or a small molecule compound.

37. The PRMT5 inhibitor for use according to claim 35 or 36, which is a small molecule compound selected from the group consisting of: GSK3326595 (pemrametostat), PF-6939999, JVNJ-64619178 (onametostat), LLY-283 and PRT543.

38. The PRMT5 inhibitor for use according to any one of claims 35 to 37, wherein administration of the PRMT5 inhibitor to a subject stimulates the subject to produce CD8+ T-cells.

39. The PRMT5 inhibitor for use according to any one of claims 35 to 38, wherein the cancer is selected from the group consisting of: leukaemia, lymphoma, multiple myeloma, lung cancer, liver cancer, breast cancer, head and neck cancer, neuroblastoma, thyroid carcinoma, skin cancer (including melanoma), oral squamous cell carcinoma, urinary bladder cancer, Leydig cell tumour, biliary cancer, such as cholangiocarcinoma or bile duct cancer, pancreatic cancer, colon cancer, colorectal cancer and gynaecological cancers, including ovarian cancer, endometrial cancer, fallopian tube cancer, uterine cancer and cervical cancer, including epithelia cervix carcinoma, optionally, the cancer is leukaemia selected from the group consisting of acute lymphoblastic leukaemia, acute myelogenous leukaemia (also known as acute myeloid leukaemia or acute non-lymphocytic leukaemia), acute promyelocytic leukaemia, acute lymphocytic leukaemia, chronic myelogenous leukaemia (also known as chronic myeloid leukaemia, chronic myelocytic leukaemia or chronic granulocytic leukaemia), chronic lymphocytic leukaemia, monoblastic leukaemia and hairy cell leukaemia.

40. The PRMT5 inhibitor for use according to claim 39, wherein the cancer is lymphoma, optionally selected from the group consisting of: Hodgkin's lymphoma; non-Hodgkin lymphoma; Burkitt's lymphoma; and small lymphocytic lymphoma.

41. The PRMT5 inhibitor for use according to claim 39, wherein the cancer is acute lymphoblastic leukaemia.
